(19) United States
(12) Patent Application Publication
(10) Pub. No.: US 2025/0352594 A1
(43) Pub. Date: Nov. 20, 2025

(54) Yeast Platform for the Production of Vaccines

(71) Applicant: SERYMUN YEAST GmbH, Bernburg (DE)

(72) Inventors: Constance MEHLGARTEN, Halle (Saale) (DE); Karin BREUNIG, Berlin (DE); Rene ZABEL, Petersberg (DE); Volker FRANKE, Burgwedel (DE); Carolin KLEINDIENST, Halle (Saale) (DE)

(21) Appl. No.: 18/036,852

(22) PCT Filed: Nov. 12, 2021
(86) PCT No.: PCT/EP2021/081604

§ 371 Date: May 12, 2023

(30) Foreign Application Priority Data
Nov. 13, 2020 (EP) 20207352.4

(51) Int. Cl. A61K 36/062; A61K 39/23; A61P 31/14; A61P 31/20; C07K 14/005; C12N 15/81

(52) U.S. Cl. CPC A61K 36/062; A61K 39/23; A61P 31/14; A61P 31/20; C07K 14/005; C12N 15/815

(57) ABSTRACT

The invention relates to the provision of recombinant yeast cells for the efficient and stable expression of transgenes, preferably for the expression of one or more immunogenic polypeptide(s) derived from a pathogen. The invention further relates to vaccine compositions comprising said recombinant yeast cells, uses of said recombinant yeast cells in methods for vaccination and 5 methods for the production of a whole yeast vaccine comprising at least one diploid recombinant yeast cell of the invention. Further encompassed are methods for the provision of a diploid yeast cell from a wild type yeast strain.

### CROSS-REFERENCE TO RELATED APPLICATIONS

This application is the U.S. National Stage application of PCT Application No. PCT/EP2021/081604, filed on Nov. 12, 2021, which claims priority to European Application No. 20207352.4, filed on Nov. 13, 2020, the contents of which are each incorporated herein by reference in their entirety.

### REFERENCE TO SEQUENCE LISTING

The instant application contains a Sequence Listing which has been submitted electronically in TXT format. The .txt file contains a sequence listing “122750_PG608US_Sequence_Listing.txt” created on Nov. 9, 2023 and is approximately 103,534 bytes in size. The sequence listing contained in this .txt file is part of the specification and is hereby incorporated by reference in its entirety.

### FIELD OF THE INVENTION

The present invention relates to recombinant yeast cells for the efficient and stable expression of transgenes, preferably for the expression of one or more immunogenic polypeptide(s) derived from a pathogen, methods of producing recombinant yeast cells and uses thereof as vaccines.

### BACKGROUND OF THE INVENTION

Viral diseases are most effectively combated by vaccines. With classical viral vaccines, a distinction is made between so-called live vaccines and dead vaccines. Live vaccines are so-called “attenuated” viruses, i.e. variants with reduced virulence, which could either be isolated as natural variants, obtained by passengers in cell culture from non-host cells, or genetically engineered by targeted mutagenesis. A characteristic feature of live vaccines is the ability of the vaccination virus to replicate, albeit at a reduced level. In contrast, dead vaccines are based on viruses that have been inactivated (e.g. by chemical or physical treatment) so that they can no longer reproduce.

Although such classical vaccines have been and are used extensively, there are problems that have encouraged the development of new vaccine types in the last decade. On the one hand, vaccinated animals cannot usually be distinguished from animals that have been infected with the field virus, and on the other hand, the production of the vaccine requires the reproduction of the virus in cell cultures or chicken eggs, which requires considerable safety precautions. There are no classical vaccines for viruses that cannot be propagated in eggs or in cell cultures, such as Porcine Rotavirus of genotype C (PRVC).

Subunit vaccines (subunit vaccines; marker vaccines) are vaccines that contain only parts of the virion (the assembled virus particle), usually structural proteins with immunogenic properties on the surface of the virus particles. Subunit vaccines are significantly safer than live vaccines and also safer than dead vaccines, since the vaccine does not contain any viral genetic material that could make replication possible. Since subunit vaccines only contain immunogenic parts of the pathogen, they usually allow the differentiation of a vaccinated animal from an animal infected with the field virus. This capability is commonly termed “DIVA capable” i.e. Differentiating between Infected and Vaccinated Animals). DIVA is of eminent importance in epidemics or monitoring programs.

Viral subunits (i.e., parts of the assembled virus particle) are naturally integrated into multimeric protein complexes. Synthesis without the further parts of the assembled virus particle, i.e., the natural interaction partners can lead to incorrect protein folding, aggregation and/or degradation and may weaken the immunogenic effect. A lower immunogenicity of individual virus components may also result from the dilution effect, which occurs when a viral antigen does not cluster on the surface of the virion to be recognized by immune cells, but as a purified, individualized protein. To counteract this dilution effect, various geometric scaffolds have been developed that can be decorated with the antigen or epitopes from it. In the simplest case, viral antigens are themselves capable of assembling into regular structures and forming so-called VLPs (virus-like particles). Such VLPs are promising approaches for the development of modern subunit vaccines.

Since the genomes of many RNA viruses are segmented, changes in the serotype are not only caused by mutations, but also by so-called reassortments of homologous RNA segments. This results in new antigen combinations, possibly with altered properties (so called emerging virus variants) and may necessitate the rapid development of new vaccines.

For a long time, both prokaryotic and eukaryotic microbes have been established as “cell factories”, as they allow comparatively inexpensive cultivation and enrichment of the desired products on an industrial scale.

Yeasts have recently proven to be very useful platforms for the development of subunit vaccines, particularly in the form of VLPs. They combine the advantages of microbial expression systems in terms of development and production costs with the advantages offered by a eukaryotic expression system. In contrast to bacteria, yeasts are eukaryotic cells which, like all animal cells, contain membrane-enveloped organelles that differ considerably from the cytoplasm in pH, redox potential, ionic strength and protein composition. Probably due to the co-evolution of viral and host proteins, many viral proteins can be better expressed or synthesized in yeasts than in bacteria. Stability and biochemical properties of proteinogenic virus components are significantly influenced by their intracellular localization.

The use of yeast cells as a protein factory usually involves the large-scale cultivation of yeast, followed by cell disruption and purification of a protein using biochemical methods. In this case, the protein synthesis capacity of the corresponding host cell is of primary importance. In addition, surface properties of the yeast cells are coming into focus. It is known that dendritic cells of the immune system can be activated by yeast cells. Yeast cells may therefore be advantageously used as a new class of vaccines, the whole yeast(-based) vaccines (WYVs) or whole recombinant yeasts (WRYs), in particular as subunit vaccines.

Characteristic for WYVs is the absence of cell disruption and purification of antigens. Whole yeast cells are inactivated and used for vaccination. One advantage of WYVs is that VLPs or other immunogenic protein complexes remain in a physiologic environment. Multiple proteins with immunogenic potential may be expressed in the same yeast cell and ideally these recombinantly expressed antigens self-assemble in the yeast cytosol forming stable virus-like particles (VLPs), subviral particles or other highly immunogenic protein complexes.

To date, little is known whether the immune response upon administration of WYVs differs from that caused by classical vaccines. It is assumed that the yeasts are taken up and destroyed by immune cells, such as dendritic cells or macrophages, so that proteins in the cytoplasm of the yeast cell can be broken down into peptides and presented by the MHC molecules.

Yeast is often used as a synonym for Saccharomyces cerevisiae. However, the term “yeast” designates unicellular fungi that lack fruiting bodies, including several thousand species belonging to taxonomic lineages that have diverged over 400 million years. Comparative genomics have revealed that the reference yeast system S. cerevisiae and its closest relatives have undergone a whole genome duplication (WGD) event followed by massive gene loss, genomic rearrangements and accelerated functional divergence of duplicated genes. Hence this clade differs in many aspects from yeasts that have not undergone the WGD, so-called pre-WGD species.

The genetic diversity among the members of the subphylum Saccharomycotina (which includes S. cerevisiae) appears to be similar to that of animals and plants. The genetic make-up determines metabolism, ecological specialization, surface properties, genetic stability, morphology and many other biological properties. For a number of yeast-based biotechnological processes, yeast species other than S. cerevisiae have proven to be superior. It is expected that the genetic differences also affect properties relevant for their use as whole cell yeast vaccines (WYV).

Among the so-called non-conventional yeasts, the genus Kluyveromyces is of particular interest for biotechnology. The two well studied Kluyveromyces species Kluyveromyces marxianus and Kluyveromyces lactis both have GRAS status (generally regarded as safe) and the ability to metabolize lactose, which is rather rare among yeasts.

Rotaviruses have a non-enveloped but relatively complex capsid structure. Therefore, the co-expression of several antigens of rotaviruses, such as the Porcine Rotavirus (PRV), in the form of VLPs is particularly challenging.

Porcine rotaviruses are the most common cause of viral gastroenteritis in young animals worldwide. Older animals can become resistant through survived infections with subsequent immunity, but also through physiological changes in the intestine. Of the 9 known rotavirus genotypes (A-I), genotypes A and C in particular occur in connection with diarrheal diseases in piglets.

Rotaviruses are double-stranded RNA viruses, which are 60 to 80 nm in size and belong to the Reoviridae family. The viruses have low host-specificity, are very resistant and cause severe gastrointestinal diseases due to their enterotoxin formation (NSP4). Rotaviruses belong to the non-enveloped RNA viruses, they are ubiquitous, almost every adult pig has undergone a rotavirus infection with corresponding antibody formation. They are among the most important pathogens in young animals and remain infectious for a long time in many environments. After oral intake, the viruses penetrate the intestinal cells (enterocytes) and destroy their resorption function.

Vaccination of pregnant animals with a so-called maternal vaccine has proven to be successful in calf rearing. Such vaccines supply the newborn with a large amount of specific rotavirus antibodies via the milk (colostrum) and may thus protect the animal from rotavirus infection in the first weeks of life. Given that there is no approved rotavirus vaccine for the vaccination of sows in various commercially important countries, there is an unmet need for an appropriate vaccine for sows against PRV.

A further viral infectious disease affecting pigs that is often associated with a severe economic impact on pig farming is caused by Porcine Parvovirus (PPV), a small, non-enveloped DNA virus with a negative, single-stranded DNA genome of about 5000 nucleotides. It was first recognized as a member of the Parvoviridae family. Worldwide it is one of the most common viral causative agents of reproductive failures called as the SMEDI syndrome (Stillbirths, Mummification, Embryonic Death, and Infertility). The incidence and severity of symptoms in sows infected with PPV virus depends on the virulence, the amount of the virus and the stage of gestation. Fetuses infected before day 70 of gestation usually die, whereas fetuses infected at a later stage of development produce antibodies against PPV, eliminate the virus and survive the infection.

Vaccination against PPV cannot prevent the virus infection and shedding, but it protects swine from SMEDI diseases. The non-pathogenic PPV-NADL-2 strain (Cluster 1) is widely used for the production of inactivated whole-virus vaccines. The relatively new vaccine ReproCyc® ParvoFLEX (Boehringer Ingelheim) is based on the virulent German field isolate PPV 27a. Here a baculovirus expression system is used to produce VP2 subunit antigens that spontaneously assemble into virus-like particles (VPLs).

Equinae (e.g., horses, zebras, or donkeys) represent another group of animal species that is farmed worldwide and is commonly affected by infectious viral diseases. One of the common viral diseases affecting equinae is AHS (African horse sickness), a highly infectious and often fatal viral infectious disease in equinae (horses, zebras and donkeys) that is transmitted by species of Culicoides (blood-sucking insects). In naïve horses, the AHS virus (AHSV) causes various forms of the disease, from a mild fever to an acute, severe form. The acute form is characterized by high fever, shortness of breath, and lethargy with a mortality rate of over 90%. AHS is endemic in tropical and sub-Saharan Africa. The distribution area seems to be expanding significantly to the north recently due to global warming. Through animal migration and the trade of infected animals on the one hand, and the spread of insects by vehicles, aircraft and strong winds on the other hand, the pathogen can be introduced into previously virus-free regions at any time. In the past, there have been regular outbreaks of AHSV in countries in North Africa, Southern Europe, as well as in the Middle East and the Arabian Peninsula. It is feared that these events will increase in the future.

AHSV is a non-enveloped virus of the genus Orbivirus with a double-stranded RNA genome containing 10 segments that encode seven structural proteins (VP1 to VP7) and five non-structural proteins NS1, NS2, NS3, NS3a and NSP4. The virus particle consists of three distinct protein layers (VP2, VP5 and VP7). VP2, the most variable protein of AHSV, is the main component of the outer capsid and the determinant of AHSV serotypes. VP2 is the serotype specific protein, and the major target of virus-neutralizing antibodies. The variability of VP2 is the basis for the antigenetic variety of the virus, which is divided into nine different serotypes (AHSV-1 to AHSV-9). In contrast, VP7 (capsid middle layer) is highly conserved between all AHSV serotypes and therefore serves as the target protein in various diagnostic AHSV assays.

### FIGURES

FIG. 1: Schematic overview of exemplary chromosomal loci for genomic integration of expression cassettes and for use as selection marker genes in yeast, in particular in K. marxianus.

FIG. 2: Schematic representation of K. marxianus mutants constructed herein. (1) ura3− (FOA selection); (2) kat1Δ::loxP-KanR-loxP/loxP (pSY15-R); (3) alpha3Δ::loxP-KanR-loxP/loxP (pSY229-R); (4) ade2Δ::ScURA3/Scura3 (pSY109-R); (5) met5Δ::loxP-KanR-loxP/loxP (pSY129-R); (6) leu2Δ::loxP-HygR-loxP/loxP (pSY241-R); (7) his3Δ::loxP-NrsR-loxP/loxP (pSY242-R); (8) lac4-lac12Δ::SCURA3/Scura3 (pSY102-R).

FIG. 3: Schematic generation of recombinant K. marxianus yeast cells with replacements on ARO10-ADE2 locus and restoration of ADE2 gene in strains with MATa-background.

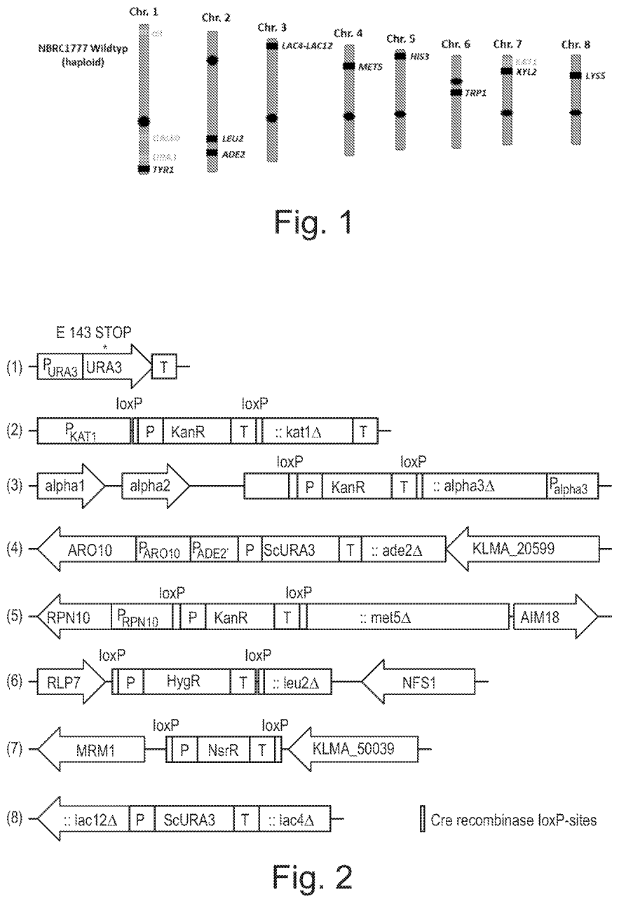

Schematic representation of the K. marxianus ARO10-ADE2 locus (1) and of the corresponding genomic region after integration of the ScURA3-based disruption cassettes indicated as ade2Δ::SCURA3 (2). The localization of the ARO10 and ADE2 genes, respectively, and the directions of transcription are indicated by arrows. For the generation of recombinant K. marxianus strains expressing two copies of PRVA-VP4-3HA-opt, the pUC19-based vector pSY147 (3) is hydrolysed with appropriate restriction enzymes (LguI+KpnI) and the linear fragment is transformed into SY267. Homologous recombination into the host genome leads to a replacement of the ScURA3 gene and the restoration of the ADE2 gene under control of its natural promoter (P_{ADE2}). Recombinant clones synthesize adenine (Ade+) and express two transgenes (e.g. RVA-VP4-3HA-opt) whose expression is controlled by one of the bidirectional promoters e.g. P_{LAC4-LAC12} and the AgTEF1 terminator (7) (e.g. strains SY277 & SY107) (4).

FIG. 4: Schematic generation of recombinant K. marxianus yeast cells with integration of intact MET5 gene for selection instead of ADE2 gene restoration in strains with MATα background.

Schematic representation of the K. marxianus ARO10-ADE2 locus (1) and of the corresponding genomic region after integration of the ScURA3-based disruption cassettes indicated as ade2Δ::SCURA3 (2). The localization of the ADE2 and ARO10 genes, respectively, and the directions of transcription are indicated by arrows. For the generation of recombinant K. marxianus strains expressing two copies of PRVA-VP2-3HA-opt, the pUC19-based vector pSY258 (3) is hydrolysed with appropriate restriction enzymes (AatII+KpnI) and the linear fragment is transformed into SY280. Homologous recombination into the host genome leads to integration of the intact MET5 gene under control of its natural promoter (P_{MET5}) instead of ADE2 gene. Recombinant clones synthesize methionine (Met+) and express two transgenes (e.g. RVA-VP2-3HA-opt) whose expression is controlled by one of the bidirectional promoters e.g. P_{LAC4-LAC12} respectively, and the AgTEF1 terminator (7) (e.g. strain SY281 & SY102) (4).

FIG. 5: Schematic generation of recombinant K. marxianus yeast cells with replacements on HIS3 locus and restoration of HIS3 gene in strains with MATα background

Schematic representation of the K. marxianus MRM1-HIS3-KLMA_50039 locus (1) and of the corresponding genomic region after integration of the Nourseothricin resistance gene (NrsR)-based deletion cassette indicated as his3Δ::NsrR (2). The localization of the MRM1, HIS3 and KLMA_50039 genes, respectively, and the directions of transcription are indicated by arrows. For the generation of recombinant K. marxianus strains expressing two copies of PRVA-VP6-3HA-opt2, the pUC19-based vector pSY250 (3) is hydrolysed with appropriate restriction enzymes (PvuI+PaeI) and the linear fragment is transformed into SY281. Homologous recombination into the host genome leads to a replacement of the NrsR gene and the restoration of the HIS3 gene under control of its natural promoter (P_{HIS3}). Recombinant clones synthesize adenine (His+) and express two transgenes (e.g. RVA-VP6-3HA-opt2) whose expression is controlled by one of the bidirectional promoters e.g. P_{LAC4-LAC12} and the AgTEF1 terminator (7) (e.g. strain SY282 & SY167) (4).

FIG. 6: Schematic generation of recombinant K. marxianus yeast cells with integration of intact LEU2 instead of HIS3 gene in strains with MATa background.

Schematic representation of the K. marxianus MRM1-HIS3-KLMA_50039 locus (1) and of the corresponding genomic region after integration of the Nourseothricin resistance gene (NrsR)-based deletion cassette indicated as his3Δ::NsrR (2). The localization of the MRM1, HIS3 and KLMA_50039 genes, respectively, and the directions of transcription are indicated by arrows. For the generation of recombinant K. marxianus strains expressing two copies of PRVA-VP7-3HA-opt, the pUC19-based vector pSY275 (3) is hydrolyzed with appropriate restriction enzymes (BglI+PaeI) and the linear fragment is transformed into SY277. Homologous recombination into the host genome leads to a replacement of the NrsR gene and integration of intact LEU2 under control of its natural promoter (P_{LEU2}) instead of HIS3 gene. Recombinant clones synthesize methionine (Leu+) and express two transgenes (e.g. RVA-VP7-3HA-opt) whose expression is controlled by one of the bidirectional promoters e.g. P_{LAC4-LAC12} and the AgTEF1 terminator (7) (e.g. strain SY278 & SY171) (4).

FIG. 7: Schematic overview of haploid K. marxianus yeast cells expressing PRVA antigens before crossing.

FIG. 8: Schematic overview of haploid K. marxianus yeast cells expressing PRVA antigens before crossing (chromosomal loci).

FIG. 9: Influence of GAL80 deletion on recombinant protein expression in K. marxianus. Western blot analyses of isogenic yeast strains with (+) and without (−) the chromosomal GAL80 gene, expressing PRVA-VP2-3HA-opt, PRVA-VP4-3HA-opt and PRVA-VP7-3HA-opt (A) or PRVA-VP6-3HA-opt (B), under control of the bidirectional KmLAC4-LAC12 promoter after cultivation on glucose and lactose.

0.3 ODU (Optical Density Units) of harvested yeast cultures were loaded onto 4-20% Mini-PROTEAN TGX Stain free gradient gels (Bio-Rad). 1 ODU corresponds to the cell mass, which gives an optical density (OD) of 1 (OD_{600 nm}=1.0) when resuspended in a volume of 1 ml. The epitope tagged proteins were detected with anti-HA (F-7, sc-7392; 1:3000, Santa Cruz Biotechnology) and HRP-conjugated goat anti-mouse secondary antibody (1:6000, Invitrogen). The antigens are indicated by arrows. Numbers below the gel lanes represent the amount of each antigen (ug per mg yeast dry weight) calculated from the band intensity using an internal standard (MalE) and quantified densitometrically by Imagelab 6.0 software (Biorad).

The deletion of the intact GAL80 gene (−) results in antigen expression on glucose, to levels that exceed the fully induced levels obtained in lactose medium.

FIG. 10: Test of immunogenicity of four PRVA antigens individually expressed in yeast strains SY102 (VP2), SY107 (VP4), SY167 (VP6) and SY171 (VP7).

(A) Immunization scheme. Eight weeks old female BALB/c mice were vaccinated subcutaneously three times with either VP2 (SY102), VP4 (SY107) and VP6 (SY167) at day 0, 14 and 28 with 2 to 5 mg yeast dry weight or two times at day 0 and 14 with 2 mg of VP7 (SY171). Control animals received a wildtype strain SY41 without antigen. Sera were collected 14 days after each vaccination for measuring seroconversion.

(B) Mouse sera were analyzed for the presence of PRVA-antigen-specific antibody against VP2, VP4, VP6 and VP7 by an IgG ELISA assay. The individual points represent individual absorbance at 450 nm. Data are representative of at least two independent experiments with similar results and average SEM value of five mice (***=p<0.001). The positive control for the ELISA was a serum of a PRVA infected pig, as negative control either zero sera before vaccination (neg. 1) or a non-specific antibody (neg. 2) was used.

After immunization with recombinant haploid yeast cells each of the strains expressing individual PRVA antigens elicited an antigen specific immune response.

FIG. 11: PCR confirmation (agarose gel electrophoresis) of ploidy status of diploid cells. Different primer sets listed in Table 6 were used to confirm the existence of both mating type alleles in the diploid strains and the integration of the transformed DNA cassettes at the expected genomic loci.

Chromosomal yeast DNA of strains (SY41=lane 1; SY278=lane 2; SY282=lane 3; SYD4=lanes 4-6) was subjected to PCR analysis using specific primer pairs verifying the ploidy state and the presence of different alleles in the haploid and diploid strain background. DNA of SY41 was used as reference for the wild type alleles.

PCR verifies that the diploid strain SYD4 contains genetic elements from both parental haploids.

TABLE 1
Overview of PCR samples subjected to agarose gel electrophoresis

| Lane | Strain | MAT | Integrations | PRVA antigens |
|---|---|---|---|---|
| 1 | SY41 | haploid; | no (ADE2, MET5, HIS3, LEU2) | — |
| | | MATa | | |
| 2 | SY278 | haploid; | ADE2::2xVP4, his3::LEU2::2xVP7, | 2x VP4, 2x VP7 |
| | | MATa | met5Δ::KanR, leu2Δ::HygR, | |
| | | | gal80Δ::Scura3, ura3- | |
| 3 | SY282 | haploid; | ade2::MET5::2xVP2, HIS3::2xVP6, | 2x VP2, 2x VP6 |
| | | MATα | met5Δ::KanR, leu2Δ::HygR, | |
| | | | gal80Δ::ScURA3 | |
| 4-6 | SYD4 | diploid; | diploid strains obtained from the | 2x VP2, 2x VP4, |
| | | MATα/MATa | mating of SY278 and SY282 | 2x VP6, 2x VP7 |

FIG. 12: Comparison of growth and recombinant protein production between haploid strains (SY278, SY282) and an isogenic diploid strain resulting from mating of the two haploids (SYD4).

(A) Growth curves for the haploid and diploid yeast strains in SD medium containing 2% glucose. Growth was measured by the optical density at 600 nm. Averages from two independent experiments are shown.

(B) Western blot quantification of VP2, VP4, VP6 and VP7 protein levels in freshly harvested yeast material after 24 h incubation. 0.3 OD (SY41), 0.15 OD (SY278), 0.075 OD (SY282) and 0.019 OD units (SYD4-K1-K3) of yeast cultures were loaded onto 4-20% Mini-PROTEAN TGX Stain free gradient gels (Bio-Rad). The HA-tagged proteins were detected with anti-HA (F-7, sc-7392; 1:3000, Santa Cruz Biotechnology) and HRP-conjugated goat anti-mouse secondary antibody (1:6000, Invitrogen). PRVA antigens are indicated by arrows. The numbers above the gel lanes represent the amount of each antigen (μg per mg yeast dry weight) calculated from the band intensities using an internal standard (MalE) and quantified densitometrically by Imagelab 6.0 software (Biorad) taking into account the loaded OD units.

It is shown that the diploid strain SYD4 expresses higher levels of VP2, VP4, VP6 and VP7 and exhibits a higher growth rate.

FIG. 13: Comparison of growth and recombinant protein production between haploid strains expressing African Swine Fever Virus (ASFV) proteins p12, p32, p62 and p72 (SY255, SY262) and the corresponding isogenic diploid strain SYD2 obtained by mating.

(A) Growth curves for the haploid and diploid yeast strains in SD medium containing 2% glucose. The optical density was measured at 600 nm and values were summed over time and averaged from two independent experiments.

(B) Western blot quantification of p12, p32, p62 and p72 protein levels in freshly harvested yeast material after 24 h incubation. 0.3 OD units of each yeast cultures were loaded onto 4-20% Mini-PROTEAN TGX Stain free gradient gels (Bio-Rad). The epitope tagged proteins were detected with anti-HA (F-7, sc-7392; 1:3000, Santa Cruz Biotechnology) and HRP-conjugated goat anti-mouse secondary antibody (1:6000, Invitrogen). ASFV antigens are indicated with arrows. The numbers above the gel lanes represent the amount of each antigen (μg per mg yeast dry weight) calculated from the band intensities using an internal standard (MalE) and quantified densitometrically by Imagelab 6.0 software (Biorad) taking into account the loaded OD units.

It is shown that the diploid strain SYD2 expresses higher quantities of p12, p32, p62 and p72 and has advantageous growth characteristics.

FIG. 14: Schematic workflow for the generation of new selectable antigen combinations by mating and sporulation of genetically marked haploid strains.

A haploid strain, in which each of 4 different chromosomes contains a different expression cassette encoding an immunogenic polypeptide coupled to a genetic marker, is crossed with another haploid strain with 4 additional antigen encoding expression cassettes. The genetic markers of the resulting diploid cell linked to the respective antigen encoding expression cassettes are displayed by an oval in the middle. Upon sporulation, the diploid cell undergoes meiosis and each spore inherits a single copy of each chromosome. Since inheritance of the MATa (white) or MATa (grey) copy occurs at random, a set of new combinations of antigen encoding expression cassettes is generated. A desired combination can be easily identified by plating on selective media due to the specific selection markers, linked to each expression cassette. In the schematic example four sets of two homologous chromosomes give 2⁴=16 combinations, where 2 of them have the genotype of the parental haploids. Hence the number of new combinations is (24)-2=14. In the case of K. marxianus having 8 chromosomes 2⁸−2=256−2=254 new combinations can be generated. The linked marker assures that selection for that marker is equivalent to selection for the linked antigen.

FIG. 15: Plasmid maps of plasmids used herein: (A) pSY16, (B) pSY102, (C) pSY109, (D) pSY129, (E) pSY147, (F) pSY241, (G) pSY242, (H) pSY250, (I) pSY258, (J) pSY275, (K) pSY123, (L) pSY131, (M) pSY278, (N) pSY279, (O) pSY15 and (P) pSY229.

FIG. 16: Immunogenicity assessment of a K. marxianus strain expressing PRVA antigens VP2, VP4, VP6 and VP7.

(A) Immunization scheme for recombinant SY320 strain. Eight weeks old female BALB/c mice (n=5) were vaccinated subcutaneously three times with SY320 (expressing PRVA VP2, VP4, VP6 and VP7) at day 0, 14 and 28 with 2 mg yeast dry weight. Control animals (n=3) received a wildtype strain (SY41) without antigen. Sera were collected 14 days after each vaccination for measuring seroconversion.

(B) and (C) ELISA assays for immunization of mice with SY320 strain. The points represent absorbance at 450 nm of individual mice sera indicating antibody induction against VP2, VP6, VP4 and VP7 42 days after vaccination. Data are representative of at least two independent experiments with similar results and average SEM value of five mice.

(D) Neutralizing antibody response were measured two weeks after the last vaccination according to the method disclosed in Kang et al., J. Clin. Microbiol. 1989, 27 (12), 2744-50.

As demonstrated by ELISA and SNT immunization with recombinant haploid yeast cells of the strain SY320, co-expressing four PRVA antigens (VP2, VP4, VP6 and VP7), induced protective neutralizing antibodies against PRVA.

FIG. 17: Immunogenicity assessment of a K. marxianus strain expressing PPV-VP2 antigen.

(A) Immunization scheme for recombinant SY61 strain. Eight weeks old female BALB/c mice (n=5) were immunized subcutaneously with a single injection of a the haploid MATa strain SY61 or a PPV-VP2 expressing diploid strain (each 2 mg yeast dry weight), respectively. Control animals received a wildtype strain SY41 without antigen (n=3). Sera collected 2 weeks post immunization were analyzed for PPV-VP2-specific IgG antibodies by an indirect ELISA.

(B) Indirect IgG ELISA assay for immunization of mice with SY61 strain. The points represent absorbance at 450 nm.

(C) Immunization scheme for recombinant SY135 strain. Eight weeks old female BALB/c mice (n=5) were immunized subcutaneously three times with SY135 (expressing two copies of PPV-VP2) or the commercial vaccines Porciles® Parvo (MSD, Tiergesundheit), respectively, at day 0, 14 and 28 with 2 mg yeast dry weight. Control animals (n=3) received a wildtype strain SY41 without antigen (n=3). Sera were collected 14 days after each vaccination for measuring seroconversion.

(D) Indirect IgG ELISA assay for immunization with SY135 strain. The individual points represent individual absorbance at 450 nm.

FIG. 18: Immunogenicity assessment of a K. marxianus strain expressing AHSV VP2 antigen.

(A) Immunization scheme for recombinant SY338 strain. Eight weeks old female BALB/c mice were vaccinated subcutaneously three times with SY338 (VP2-serotype 4) at day 0, 14 and 28 with 2 mg yeast dry weight (n=5). Control animals received a wildtype strain SY41 without antigen (n=3).

(B) Neutralization titer at day 42. Neutralizing antibodies were determined by plaque reduction in presence of 100 TCID of virus based standard neutralizing assay. The neutralization titres were calculated by the reciprocal value of the maximum dilution, at which the number of plaques showed 50% reduction compared with the serum-free control. The neutralizing tests were performed in duplicate.

### DETAILED DESCRIPTION OF THE INVENTION

The present invention relates to recombinant yeast cells for highly efficient expression of transgene-encoded gene products, such as immunogenic polypeptides, methods of producing recombinant yeast cells and uses of recombinant yeast cells as vaccines.

A recombinant yeast cell according to the invention comprises at least one genomically integrated expression cassette, wherein each expression cassette comprises (i) a bidirectional promoter element; (ii) a first transgene and a second transgene, wherein said first and second transgene are located at opposite ends of the bidirectional promoter element and wherein each transgene is operably linked to one side of the bidirectional promoter element; (iii) a first transcription terminator and a second transcription terminator, said first transcription terminator being located immediately downstream of the first transgene and said second transcription terminator being located immediately downstream of the second transgene; wherein the first transcription terminator is operably linked to the first transgene and the second transcription terminator is operably linked to the second transgene; and (iv) at least one selection marker.

The term “recombinant” as used herein refers to “being prepared by or the result of genetic engineering”. Thus, a recombinant microorganism or host cell (e.g., a yeast cell) comprises at least one “recombinant nucleic acid”, i.e., a nucleic acid that has been prepared by or is the result of genetic engineering. A recombinant yeast cell specifically comprises an expression cassette that has been stably integrated into the yeast cell genome by means of genetic engineering.

The term “expression cassette” refers to nucleic acid molecules containing desired coding sequences (transgenes) and control sequences in operable linkage, so that host cells transformed with these sequences are capable of producing the polypeptides encoded by the transgenes. In order to obtain genetic stability, the expression cassette is integrated into the host genome (i.e., it is genomically integrated), for example by homologous recombination.

The recombinant yeast cell according to the invention may comprise more than one genomically integrated expression cassette. In one embodiment, recombinant yeast cell comprises at least two genomically integrated expression cassettes. For example, the recombinant yeast cell according to the invention may comprise 2, 3, 4, 5, 6, 7 or 8 genomically integrated expression cassettes. Preferably, the recombinant yeast cell comprises 2, 4 or 6 genomically integrated expression cassettes. In a particularly preferred embodiment, the recombinant yeast cell comprises 2 genomically integrated expression cassettes. In another particularly preferred embodiment, the recombinant yeast cell comprises 4 genomically integrated expression cassettes. In yet another particularly preferred embodiment, the recombinant yeast cell comprises 6 genomically integrated expression cassettes.

The expression cassette according to the invention specifically comprises a bidirectional promoter element, operably linked to coding regions of nucleotide sequences located on both sides of the promoter, in opposite directions, i.e., a first transgene and a second transgene, each under the transcriptional control of said bidirectional promoter element. The bidirectional promoter element is not natively associated with the transgenes.

The term “transgene” as used herein shall refer to any coding gene, e.g. encoding a protein of interest (POI), including polypeptides. The terms “protein” and “polypeptide” are used herein interchangeably. A transgene encodes a protein not naturally occurring in the host cell, i.e. a heterologous protein. The protein of interest can be expressed upon integration by recombinant techniques of one or more copies of the nucleic acid sequence encoding the protein of interest into the genome of the host cell.

In a preferred embodiment, the nucleic acid sequence of the transgene has been codon optimized. The term “codon-optimized” refers to adaption in the design of a coding sequence to the codon usage in the host cell, which often improves the translation efficiency of the protein of interest in the chosen expression system (e.g., the recombinant yeast cell). By virtue of the degeneracy of the genetic code, the translation of a nucleotide sequence into an amino acid sequence is unambiguous whereas for the “reverse translation” of an amino acid sequence into a nucleotide sequence there are several options because some amino acids are encoded by more than one codon. Highly expressed proteins tend to be encoded by codons that make use of abundant tRNA species. Since even closely related organisms may differ in the set of isoaccepting tRNA genes in their genomes, algorithms for codon optimization take advantage of genomic information on the set of isoaccepting tRNA genes and the codon usage in highly expressed genes of the host.

The transgenes may encode various proteins of interest. In one embodiment, each transgene encodes an immunogenic polypeptide derived from a pathogen, or an immunogenic fragment thereof. The pathogen may be a bacterial pathogen, a viral pathogen, a fungal pathogen, a protozoan pathogen, or a multi-cellular parasitic pathogen. Preferably, the pathogen is a viral pathogen. Exemplary viral pathogens include viral pathogens affecting livestock, such as ASFV (African Swine Fever Virus), PPV (Porcine Parvovirus), PRV (Porcine Rotavirus), FMDV (Foot and Mouth Disease Virus), BTV (Bluetongue Virus), PEDV (Porcine Epidemic Diarrhea Virus), PRRSV (Porcine Respiratory and Reproductive Syndrome Virus), PPRV (Peste des Petits Ruminants Virus), RVFV (Rift Valley Fever Virus). Further exemplary viral pathogens include those occurring in aquaculture, such as IPNV (Infectious Pancreatic Necrosis Virus), HSMIV (Heart Skeletal Muscle Inflammation Virus), PDV (Pancreas Disease Virus), ISA (Infectious Salmon Anemic Virus), CyHV-2 (Cyprinid Herpesvirus 2) as well as viral pathogens relevant in companion animals, e.g., AHSV (African Horse Sickness Virus), WNV (West Nile Virus), FIV (Feline Immunodeficiency Virus), EVA (Equine Viral Arteritis). In a particularly advantageous embodiment, the viral pathogen is a viral pathogen with a multilayer capsid and one or more spike proteins.

As used herein, the term “immunogenic” or “immunogenicity” refers to the property of polypeptides, nucleic acids, or other components of a pathogen of raising an immune response in a host cell. A molecular structure (e.g., a polypeptide chain) with a specific shape and/or a short peptide with a specific sequence that is recognized by the immune system (antibodies, B cells and/or T cells) of an animal and elicits an immune response is termed epitope. The immune response of the host organism can be specific or non-specific. In some cases, an immunogenic polypeptide of a pathogen results in both, a non-specific and a specific immune response. Preferably, the immune response comprises an immune response that is specific for the immunogenic polypeptide, or immunogenic polypeptides that are encoded by each transgene comprised in the at least one genomically integrated expression cassette. Further preferably, the immune response is protective against the pathogen from which the immunogenic polypeptide has been derived. An immune response is protective if it prevents or attenuates a disease or condition arising from infection of a host organism or patient with the pathogen, or pathogens that an immunogenic polypeptide has been derived from.

In some embodiments, the transgenes encode immunogenic polypeptides derived from more than one pathogen, or an immunogenic fragment thereof. In one embodiment, the transgenes encode immunogenic polypeptides derived from more than one strain of the same viral pathogen, or an immunogenic fragment thereof. In another embodiment, the transgenes encode immunogenic polypeptides derived from more than one viral pathogen, or an immunogenic fragment thereof.

Apart from the native coding sequence and/or a codon optimized variant thereof, the transgenes may encode further variants of the native immunogenic polypeptide. The variants of the native immunogenic polypeptide essentially retain or increase the immunogenicity of the native form of the immunogenic polypeptide. Such variants include amino acid (aa) substitutions, additions or deletions. The coding DNA sequence can be designed by reverse translation and is introduced into an expression cassette as a synthetic piece of DNA or by modifying the DNA of an existing genetic locus. Generally, the modification may serve a function including but not restricted to elimination of a post-transcriptional modification site (e.g. glycosylation), prevention of misfolding or aggregation, as well as enhancement of immunogenicity. Variant immunogenic polypeptides also include fragments of the polypeptides disclosed herein, particularly immunogenic fragments. Also encompassed are immunogenic polypeptides that have been modified to improve resistance to proteolytic degradation, increase solubility or render them more suitable as an immunogen, or antigenic component of a vaccine composition. Further variants include fusion polypeptides comprising an immunogenic polypeptide and a functional fragment changing the intracellular localization of the fusion polypeptide, or an epitope tag.

In a specific embodiment, the pathogen is a viral pathogen that belongs to the family of Reoviridae. Preferably, the viral pathogen is a rotavirus. For example, rotavirus A, B, C, D, E, F, G, H or I. More preferably, the viral pathogen is a Porcine Rotavirus, such as Porcine Rotavirus A, B or C. Even more preferably, the viral pathogen is Porcine Rotavirus A (PRVA) or Porcine Rotavirus C (PRVC), most preferably Porcine Rotavirus A (PRVA). In another preferred embodiment, the viral pathogen is an orbivirus, most preferably African Horse Sickness Virus (AHSV) or Bluetongue virus (BTV). In yet another preferred embodiment, the viral pathogen is Porcine Parvovirus (PPV).

In a particularly preferred embodiment, each immunogenic polypeptide comprises a polypeptide selected from the group consisting of Porcine Rotavirus A (PRVA) VP2, VP4, VP6, VP7, NSP2 and NSP4 and one or more immunogenic fragment thereof, most preferably from the group consisting of Porcine Rotavirus A (PRVA) VP2, VP4, VP6 and VP7 and one or more immunogenic fragment thereof. In a specific example, each immunogenic polypeptide is derived from Porcine Rotavirus A strain RVA/Pig-tc/ESP/OSU-C5111/2010/G5P [7]. For example, each immunogenic polypeptide comprises a polypeptide selected from the group consisting of SEQ ID NO: 1 (PRVA VP2), SEQ ID NO: 2 (PRVA VP4), SEQ ID NO: 3 (PRVA VP6), SEQ ID NO: 4 (PRVA VP7), SEQ ID NO: 5 (PRVA NSP2) and SEQ ID NO: 6 (PRVA NSP4) and one or more immunogenic fragment thereof, preferably from the group consisting of SEQ ID NOS: 1, 2, 3, and 4 and one or more immunogenic fragment thereof.

The nucleotide sequences encoding PRVA VP2, VP4, VP6, VP7, NSP2 and NSP4 are set forth in SEQ ID NO: 7 (PRVA VP2, GenBank accession number: KJ450843.1), SEQ ID NO: 8 (PRVA VP4, GenBank accession number: KJ450845.1), SEQ ID NO: 9 (PRVA VP6, GenBank accession number: KJ450847.1), SEQ ID NO: 10 (PRVA VP7, GenBank accession number: KJ450849.1), SEQ ID NO: 11 (PRVA NSP2, GenBank accession number: KJ450850.1) and SEQ ID NO: 12 (PRVA NSP4, GenBank accession number: KJ450851.1).

Advantageously, the nucleotide sequences are codon optimized for expression in yeast. Preferred codon optimized nucleotide sequences encoding PRVA VP2, VP4, VP6, VP7, NSP2 and NSP4, are set forth in SEQ ID NO: 13 (PRVA VP2), SEQ ID NO: 14 (PRVA VP4), SEQ ID NO: 15 (PRVA VP6), SEQ ID NO: 16 (PRVA VP7), SEQ ID NO: 17 (PRVA NSP2) and SEQ ID NO: 18 (PRVA NSP4). Optionally, the immunogenic polypeptides comprise a peptide tag, such as an HA tag. Exemplary amino acid sequences, each comprising an HA tag are set forth in SEQ ID NO: 19 (PRVA VP2), SEQ ID NO: 20 (PRVA VP4), SEQ ID NO: 21 (PRVA VP6), SEQ ID NO: 22 (PRVA VP7), SEQ ID NO: 23 (PRVA NSP2) and SEQ ID NO: 24 (PRVA NSP4).

Thus, in a particularly preferred embodiment, each immunogenic polypeptide comprises a polypeptide selected from the group consisting of Porcine Rotavirus A (PRVA) VP2, VP4, VP6 and VP7 and one or more immunogenic fragment thereof, wherein the nucleotide sequences encoding Porcine Rotavirus A (PRVA) VP2, VP4, VP6 and VP7 are set forth in SEQ ID NO: 13 (PRVA VP2), SEQ ID NO: 14 (PRVA VP4), SEQ ID NO: 15 (PRVA VP6) and SEQ ID NO: 16 (PRVA VP7). In a highly preferred embodiment, each immunogenic polypeptide further comprises an HA tag and consists of an amino acid sequence selected from the group consisting of SEQ ID NO: 19 (PRVA VP2), SEQ ID NO: 20 (PRVA VP4), SEQ ID NO: 21 (PRVA VP6) and SEQ ID NO: 22 (PRVA VP7).

In a further specific embodiment, the pathogen is a viral pathogen that belongs to the family of Parvoviridae. Preferably, the viral pathogen is a Protoparvovirus, more preferably Porcine Parvovirus (PPV), even more preferably Ungulate protoparvovirus 1 (also referred to as Porcine Parvovirus 1, PPV1) or Ungulate protoparvovirus 2 (PPV2). Even more preferably, the viral pathogen is PPV1. Exemplary PPV1 strains include PPV1 strain 27a (AY684871.1), PPV1 strain NADL-2 (L23427.1), PPV1 strain Kresse (U44978.1), PPV1 strain143a (AY684867.1), PPV1 strain WB631 (JQ249917.1), PPV1 strain CC7 (MH091023) and PPV1 strain Campinas (AY145500.1) and strains comprising immunogenic polypeptides having an amino acid sequence that is at least 90%, at least 92%, at least 94%, at least 96%, at least 98% or at least 99% identical with the amino acid sequences of the immunogenic polypeptides of any one of these strains.

In a particularly preferred embodiment, each immunogenic polypeptide comprises a polypeptide selected from the group consisting of Porcine Parvovirus (e.g., PPV1) VP1, VP2, NS1 and NS2 and one or more immunogenic fragment thereof. Most preferably, each immunogenic polypeptide comprises a Porcine Parvovirus (e.g., PPV1) VP2 polypeptide or one or more immunogenic fragments thereof.

The PPV VP2 protein is closely related to the virus-host range and antigenicity. It is generally considered as the major immunogenic antigen of PPV vaccines since it contains most of the B-cell epitopes critical to elicit neutralizing antibodies. In vitro expressed PPV VP2 protein can spontaneously self-assemble into VLPs to form the capsid and to mimick the morphology of a pathogenic virus. PPV VLPs produced in K. marxianus were observed to elicit high titres of lgG antibodies and hemagglutination inhibition antibody.

In yet another specific embodiment, the pathogen is African Horse Sickness Virus (AHSV). For example, any of the serotypes AHSV-1, AHSV-2, AHSV-3, AHSV-4, AHSV-5, AHSV-6, AHSV-7, AHSV-8 or AHSV-9. In a preferred embodiment, the AHSV is AHSV-4 (i.e., AHSV serotype 4). Preferably, each immunogenic polypeptide comprises a polypeptide selected from the group consisting of African Horse Sickness Virus (AHSV) VP1, VP2, VP3, VP4, VP5, VP6, VP7, NS1, NS2, NS3, NS3a and NS4 and one or more immunogenic fragment thereof. More preferably, each immunogenic polypeptide comprises a polypeptide selected from the group consisting of African Horse Sickness Virus (AHSV) VP2, VP3, VP5 and VP7 and one or more immunogenic fragment thereof. Even more preferably, the immunogenic polypeptide comprises a polypeptide selected from the group consisting of African Horse Sickness Virus (AHSV) VP2 and one or more immunogenic fragment thereof. Most preferably, the immunogenic polypeptide comprises a polypeptide selected from the group consisting of African Horse Sickness Virus serotype 4 (AHSV-4) VP2 and one or more immunogenic fragment thereof.

In yet another embodiment, the pathogen is African Swine Fever Virus (ASFV), for example ASFV isolate Georgia/2007 (GenBank: FR682468.1). Preferably, each immunogenic polypeptide comprises a polypeptide selected from the group consisting of African Swine Fever Virus (ASFV) proteins p12, p32, p62 and p72.

In one embodiment, the recombinant yeast cell comprises two genomically integrated expression cassettes, wherein the first and second transgene of each expression cassette encode one immunogenic polypeptide derived from a pathogen. In a further embodiment, the recombinant yeast cell comprises three genomically integrated expression cassettes, wherein the first and second transgene of each expression cassette encode one immunogenic polypeptide derived from a pathogen. In an even further embodiment, the recombinant yeast cell comprises four genomically integrated expression cassettes, wherein the first and second transgene of each expression cassette encode one immunogenic polypeptide derived from a pathogen.

Each transgene may encode an immunogenic polypeptide (or immunogenic fragment thereof) that is derived from a viral pathogen (e.g., PRVA). In one embodiment, the immunogenic polypeptide, or polypeptides, are capable of assembling into a virus-like particle (VLP) inside the recombinant yeast cell. This means that the immunogenic polypeptide(s) is/are a structural component of the capsid of a viral pathogen and thus capable of taking part in the assembly of a VLP, optionally in combination with further immunogenic polypeptides. Said further immunogenic polypeptides may be derived from the same viral pathogen, or from a further viral pathogen that is closely related with the first viral pathogen. In the case of viral pathogens with a simple symmetric capsid, one major structural protein of the virus can form virus-like particles (VLPs) when expressed recombinantly in a yeast cell.

The homo- or hetero-multimerization of one or more structural proteins into complex VLPs inside a recombinant yeast was found to be highly beneficial for the development of whole yeast vaccines. For structurally simple viral pathogens, where the capsid is composed of a single layer of a single structural protein, it has been confirmed that the immune response of the host organism is substantially enhanced when the structural protein has assembled into VLPs. However, the capsid structure of many viruses is composed of more than one protein in more than one layer, and more than one of these structural proteins possesses individual immunogenic properties.

Rotavirus has a complex triple-layered capsid structure composed of VP2 forming the innermost layer, VP6 forming the middle layer and VP4 and VP7 as parts of the outer layer. Co-expression of three of these antigens, VP2, VP6 and VP7, in Saccharomyces cerevisiae has been shown to yield rotavirus-VLPs (RLPs). However, the antigens were expressed from one or more plasmid(s), which is cumbersome and leads to low and unpredictable antigen expression.

A prerequisite for VLP formation from a single immunogenic polypeptide in yeast is the ability of that polypeptide to associate spontaneously into an essentially regular geometrical structure. One example is the capsid protein of parvovirus. Generally, many viral capsid structures are more complex (e.g., the capsid of rotavirus). They can form multimeric protein complexes composed of several different structural polypeptides, optionally associated with non-structural proteins that may or may not be immunogenic. The possibility to form multi-component VLPs in yeast has been shown to require the coordinate expression of several transgenes encoding the required polypeptides in a single cell. Co-expression is greatly facilitated by employing the bidirectional promoter elements and genomically integrated expression cassettes according to the invention. The invention enables accumulation of the VLP components at high concentrations in the recombinant yeast cell, a prerequisite for efficient assembly of the often highly immunogenic VLP particles.

Virus Like Particles (VLPs) generally contain one or more proteins from a virus optionally combined with a phospholipid. VLPs are generally non-pathogenic, non-replicating and generally do not contain any of the native viral genome. Since VLPs cannot replicate, they provide a safer alternative to attenuated viruses for vaccine development or vaccination. The VLPs may be produced in the recombinant yeast cell of the invention. Due to their virosome-like structure, VLPs contain repetitive, high density displays of viral surface proteins that present conformational viral epitopes which can elicit strong T cell and B cell immune responses. The small radius of common VLPs (generally 20-200 nm) allows for sufficient draining into lymph nodes.

The recombinant yeast cell comprises at least one genomically integrated expression cassette, wherein each expression cassette comprises a first and a second transgene. Each expression cassette may comprise different combinations of transgenes. In one embodiment, the first transgene and the second transgene are identical within each expression cassette. In another embodiment, the first transgene and the second transgene are different within each expression cassette.

The recombinant yeast cell may comprise one genomically integrated expression cassette, or more than one genomically integrated expression cassette. In one embodiment, the recombinant yeast cell comprises at least two genomically integrated expression cassettes (e.g., 2, 3, 4, 5, 6, 7 or 8 genomically integrated expression cassettes) and the first transgene and the second transgene of each expression cassette are different from the first and second transgene of each further expression cassette. Thus, in a specific embodiment of the invention, the first transgene and the second transgene of each expression cassette are different from the first and second transgene of each further expression cassette and the first transgene and the second transgene are identical within each expression cassette. In another specific embodiment, the first transgene and the second transgene of each expression cassette are different from the first and second transgene of each further expression cassette and the first transgene and the second transgene are different within each expression cassette.

The recombinant yeast cell may comprise transgenes that are identical to each other. Alternatively, the recombinant yeast cell may comprise transgenes that are different from each other. Accordingly, in one embodiment, the recombinant yeast cell comprises one type of transgene. In a further embodiment, the recombinant yeast cell comprises at least two different transgenes. In a further embodiment, the recombinant yeast cell comprises at least three different transgenes. In a further embodiment, the recombinant yeast cell comprises at least four different transgenes. In a further embodiment, the recombinant yeast cell comprises at least five different transgenes. In a further embodiment, the recombinant yeast cell comprises at least six different transgenes. In a further embodiment, the recombinant yeast cell comprises at least seven different transgenes. In a further embodiment, the recombinant yeast cell comprises at least eight different transgenes. In a further embodiment, the recombinant yeast cell comprises at least nine different transgenes. In a further embodiment, the recombinant yeast cell comprises at least ten different transgenes.

In a specific embodiment, the recombinant yeast cell comprises one copy of each transgene. In a further embodiment, the recombinant yeast cell comprises two copies of each transgene. In a further embodiment, the recombinant yeast cell comprises three copies of each transgene. In a further embodiment, the recombinant yeast cell comprises four copies of each transgene. In a further embodiment, the recombinant yeast cell comprises five copies of each transgene. In a further embodiment, the recombinant yeast cell comprises six copies of each transgene. In a further embodiment, the recombinant yeast cell comprises seven copies of each transgene. In a further embodiment, the recombinant yeast cell comprises eight copies of each transgene.

Preferably, the recombinant yeast cell comprises at least two copies of each transgene. In a further preferred embodiment, the recombinant yeast cell comprises at least three copies of each transgene. In yet another preferred embodiment, the recombinant yeast cell comprises at least four copies of each transgene. In yet another preferred embodiment, the recombinant yeast cell comprises at least five copies of each transgene. In yet another preferred embodiment, the recombinant yeast cell comprises at least six copies of each transgene. In yet another preferred embodiment, the recombinant yeast cell comprises at least seven copies of each transgene. In yet another preferred embodiment, the recombinant yeast cell comprises at least eight copies of each transgene.

To date, the choice of promoters for heterologous gene expression, especially for eukaryotic organisms, such as yeast, has focussed on monodirectional promoters. The assembly of VLPs or the coverage of diverging serotypes/genotypes of pathogenic viruses often requires the simultaneous expression of numerous immunogenic polypeptides and variants thereof in one (yeast) cell. Classically, monodirectional promoters have been used for such applications, but monodirectional promoters are limited in terms of multi-gene co-expression capabilities. Furthermore, each individual chromosomal integration of an insert comprising a monodirectional promoter and one transgene requires (and “consumes”) one selection marker, with only a limited number of such marker genes being available. The use of bidirectional promoter elements necessitates only one selection marker gene per pair of transgenes.

Additionally, bidirectional promoter elements offer the ability to double the number of transgenes inserted into the genome of a host cell in one transformation step, which reduces the effort for verification of the correct recombination events significantly. The bidirectional promoter elements allow the coordinate expression of two transgenes, and thus of two heterologous proteins from one control region. A major advantage of using the bidirectional promoters is a shielding effect against influences of flanking sequences on promoter activity. The chromatin of promoter elements is generally more open allowing access of DNA binding proteins whereas protein coding sequences of a transcription unit are generally organised in a regular nucleosome array. Fusing a promoter element to a region with extremely open or closed chromatin structure runs the risk of having an unwanted influence of the flanking region on the activity of the selected promoter element. This well documented phenomenon of insertion site specific influence on the expression of genes randomly integrated into the eukaryotic genome is reduced, at least in yeast, when protein coding sequences are flanking both sides of a bidirectional promoter. In addition, bidirectional promoters are also reducing the cloning efforts for expression cassettes.

Moreover, mating haploid recombinant yeast cells comprising such expression cassettes at different selected positions in the yeast genome substantially speeds up the development of multivalent whole yeast vaccines, since the number of antigens increases by the factor two with each integration.

For example, a haploid recombinant yeast cell according to the invention, comprising two genomically integrated expression cassettes can be generated through only two transformation steps. This recombinant yeast cell may encode up to four different transgenes, e.g., four different immunogenic polypeptides. Mating of said haploid recombinant yeast cell with a further haploid recombinant yeast cell, which also comprises two expression cassettes, each comprising two transgenes would result in a diploid recombinant yeast cell comprising eight transgenes, encoding up to eight different immunogenic polypeptides.

Generally, a bidirectional promoter element is located within the intergenic region between the 5′ ends of a bidirectional gene pair. A “bidirectional gene pair” refers to two adjacent and divergently (as opposed to convergently) transcribed genes encoded on opposite strands. Natively, the two genes are often functionally related, and their shared upstream region allows them to be co-regulated and thus co-expressed. Bidirectional promoter elements enable efficient and controllable expression of two transgenes located at opposite ends of the bidirectional promoter element wherein each transgene is operably linked to one side of the bidirectional promoter element.

Generally, the term “promoter element” as used herein refers to a DNA sequence capable of controlling the expression of a protein coding sequence. It consists of a segment that determines the start site(s) and the direction of transcription for each transcription unit and additional elements that contribute to control the rate of transcription and thus the expression level of a gene operably linked to the promoter by serving as binding sites for regulatory factors. Such regulatory factors influence the promoter activity in response to extracellular or intracellular conditions. As used herein, the terms “transcription rate” and “transcription level” are used interchangeably. Regulatory sequences of bidirectional promoters can cooperate and act on the two core promoter elements driving divergent transcription, as in the case of the GAL1-GAL10 or LAC4-LAC12 promoters or act unidirectionally on only one of the two core promoters. These different types of regulatory elements can co-exist in the same bidirectional promoter element. In the expression cassettes constructed in this invention the fusion point for the protein coding region of the transgene and the promoter element is at or close to the translation start codon.

The term “operably linked” (or “cis-acting”) as used herein refers to the association of nucleotide sequences on a single nucleic acid molecule, e.g. an expression cassette or a vector, in a way such that the function of one or more nucleotide sequences is affected by at least one other nucleotide sequence present on said nucleic acid molecule. For example, a promoter element is operably linked with a coding sequence of a transgene, when it is capable of effecting the expression of that coding sequence. Operable linking is accomplished by ligation of polynucleotides at convenient restriction sites or by PCR and recombination methods familiar to those skilled in the art, that allow seamless fusion of DNA fragments, such as In-Fusion Cloning (Zhu, Baogong; Cai, Guifang; Hall, Emily O.; Freeman, Gordon J. (2007), Bio Techniques 43 (3), S. 354-359).

Since the core eukaryotic transcription apparatus is conserved, promoter DNA and coding DNA may be from the same or from different organisms. Preferably, the bidirectional promoter element comprised in the genomically integrated expression cassette according to the invention is a native bidirectional yeast promoter element. This means that the bidirectional promoter element naturally or natively occurs as a bidirectional promoter element that controls transcription of a homologous gene pair in yeast. In a specific embodiment, the native bidirectional yeast promoter element is evolutionarily conserved in at least three divergently evolved yeast genera (Byrne, K. P. (2005), Genome Research 15 (10), p. 1456-1461; see yeast gene order browser, http://ygob.ucd.ie). In a further specific embodiment, the native bidirectional yeast promoter element is derived from the yeast genus Kluyveromyces, more specifically from the yeast species Kluyveromyces marxianus.

In an advantageous embodiment, the native bidirectional yeast promoter element is selected from the group consisting of a LAC4-LAC12 promoter, a GAL1-GAL10 promoter and a GAP1-ADH2 promoter. In a further advantageous embodiment, the native bidirectional yeast promoter element is selected from the group consisting of a K. marxianus LAC4-LAC12 promoter, a K. marxianus GAL1-GAL 10 promoter and a K. marxianus GAP1-ADH2 promoter.

Further advantageous native bidirectional yeast promoter elements are listed in Table 2 (Eriksson, Peter R.; Ganguli, D.; Nagarjavel, V.; Clark, David J. (2012), Genetics 191(1): 7-20; Nishizawa, M; Komai, T. et al. (2008), Eukaryotic Cell 7(6), 949-957; Hu, Haiyan; Li, Xiaoman (2007), Genomics 90(4), 421-423).

TABLE 2
Preferred native bidirectional yeast promoter elements.

| Promoter | Organism | Class | Induction | Repression |
|---|---|---|---|---|
| P_{LAC4-LAC12} | K. marxianus | regulated | Galactose/Lactose | Glucose |
| P_{LAC4-LAC12} | K. lactis | | | |
| P_{GAL1-GAL10} | K. marxianus | regulated | galactose | Glucose |
| P_{GAL1-GAL10} | K. lactis | | | |
| P_{GAL1-GAL10} | S. cerevisiae | | | |
| P_{GAP1-ADH2} | K. marxianus | P_{GAP1} - constitutive | No | |
| | | P_{ADH2} - regulated | Ethanol- & stationary phase- | |
| | | | dependent regulated- | |
| P_{HTA1-HTB1} | K. marxianus | regulated | cell cycle regulated | |
| P_{HTA1-HTB1} | K. lactis | | | |
| P_{HTA1-HTB1} | S. cerevisiae | | | |
| P_{HHT1-HHF1} | K. marxianus | regulated | cell cycle regulated | |
| P_{HHT1-HHF1} | K. lactis | | | |
| P_{HHT1-HHF1} | S. cerevisiae | | | |
| P_{HHT2-HHF2} | K. marxianus | regulated | cell cycle regulated | |
| P_{HHT2-HHF2} | K. lactis | | | |
| P_{HHT2-HHF2} | S. cerevisiae | | | |
| P_{SNO3-SNZ3} | K. marxianus | regulated | stationary phase-induced | |
| P_{SNO1-SNZ1} | K. lactis | | | |
| P_{SNO1-SNZ1} | S. cerevisiae | | | |
| P_{RPL15B-KLMA}_{_{—}}₄₀₀₇₈ | K. marxianus | regulated | various cellular | |
| P_{RPL15A-KLLAO}_{_{—}}_{F17611g} | K. lactis | | states-regulated | |
| P_{RPL15A-YLR030w} | S. cerevisiae | | | |

These native bidirectional yeast promoter elements include the GAL1-GAL10 (P_{GAL1-GAL10}) promoter element (in short: GAL promoter) of S. cerevisiae (nucleotides 278744 to 279411 of the sequence according to NCBI accession number CP020124), K. lactis (nucleotides 781010 to 781507 of the sequence according to NCBI accession number CR382126) and K. marxianus (SEQ ID NO: 25) and the LAC4-LAC12 (P_{LAC4-LAC12}) promoter element (in short: LAC promoter) of K. lactis (nucleotides 1307691 to 1310500 of the sequence according to NCBI accession number CR382122) and K. marxianus (SEQ ID NO: 26), which are regulated by the transcription activator Gal4p. In a preferred embodiment, the native bidirectional yeast promoter element comprises a nucleic acid sequence selected from the group consisting of SEQ ID NOs 25 and 26.

The GAL and LAC genes that are naturally regulated by these promoter elements are required for the metabolism of the sugars galactose and lactose (a galactose-glucose disaccharide). The consensus sequence for the binding site (5′-CGGnnnA/TnnnCCG-3′) within the GAL and LAC bidirectional promoter elements is conserved, indicating that the specificity of the DNA-binding domain of the homologous Gal4p variants, e.g., ScGal4p, KIGal4p (also named Lac9) and KmGal4, is also conserved. The expression of the native gene pairs under the control of the GAL1-GAL10 and LAC4-LAC12 promoter elements is co-ordinately regulated by the Gal4p-regulated bidirectional promoters and can be induced by supplementation of the growth medium with galactose or lactose. Likewise, two (or four) transgenes linked to these bidirectional promoter elements can be co-ordinately regulated by Gal4p

In glucose medium, the LAC and GAL promoter elements are silent, which is beneficial when the antigen expression leads to protein-stress in the recombinant yeast cell. Accordingly, recombinant yeast cells can first be cultivated on glucose medium for biomass accumulation before lactose is added to activate the LAC4-LAC12 promoter and transgene expression is induced. Transcriptional repression of the LAC and GAL promoter elements requires the regulatory protein Gal80p, which can block the transcription activation function of Gal4p by direct protein-protein interaction. The regulatory network regulating the LAC/GAL promoters operates one of the most effective eukaryotic transcriptional switches known. On the one hand Gal4p controlled gene expression can be modulated by a small nutrient, galactose, on the other hand it can be modulated by the ratio of its regulators Gal80p and Gal4p. The inventors have surprisingly found that, when transgenes are linked to the bidirectional LAC and GAL promoter elements, the intracellular concentration of multiple transgene-encoded proteins, such as PRVA antigens, can be elevated substantially even beyond the fully induced levels upon deletion of the KmGAL80 gene.

Thus, in a particularly advantageous embodiment, the bidirectional promoter element is selected from a LAC4-LAC12 promoter or a GAL1-GAL10 promoter and the recombinant yeast cell comprises a deletion and/or disruption of GAL80. If the recombinant yeast cell comprises more than one genomically integrated expression cassette (i.e., at least two genomically integrated expression cassettes), each cassette may comprise a LAC4-LAC12 promoter, each cassette may comprise a GAL1-GAL10 promoter, or the expression cassettes may comprise either a LAC4-LAC12 or a GAL1-GAL10 promoter element and the recombinant yeast cell comprises a deletion and/or disruption of GAL80.

Also encompassed by the invention are native bidirectional yeast promoter elements that have been adapted to increase transgene expression, or to enable expression control using a different stimulus or to give high constitutive promoter activity (adapted native bidirectional yeast promoter elements).

In a specific embodiment, the bidirectional promoter element is a hybrid bidirectional promoter element. Such hybrid promoter elements are composed of sections from different monodirectional native (natural) yeast or heterologous promoter elements, preferably from different monodirectional natural yeast promoter elements. Said monodirectional promoter elements may natively be part of a bidirectional promoter element. Both monodirectional promoter elements are selected such that they do not interfere with each other. The different monodirectional promoter elements are fused in head-to-head fashion, to control bidirectional transcription of the linked genes. For example, hybrid promoter elements for use in S. cerevisiae (and potentially other yeast species) can be constructed by combining heterologous regulatory promoter motifs with homologous core promoters (Hubmann, Georg; Thevelein, Johan M.; Nevoigt, Elke (2014), Methods in molecular biology (Clifton, N.J.) 1152, S. 17-42). Efficient and robust recombinant protein expression by hybrid-regulated bidirectional promoter elements has already been described in the literature for some yeast species, e.g., P_{TDH3-ADH1} (Miller, C. A.; Martinat, M. A.; Hyman, L. E. (1998), Nucleic acids research 26 (15), S. 3577-3583); P_{GAL1 or GAL10-GPD} (Li, Aimin; Liu, Zengshan; Li, Qianxue; Yu, Lu; Wang, Dacheng; Deng, Xuming (2008), FEMS yeast research 8 (1), S. 6-9); P_{TEF1-PGK1} (Partow, Siavash; Siewers, Verena; Bjørn, Sara; Nielsen, Jens; Maury, Jérôme (2010), Yeast (Chichester, England) 27 (11), S. 955-964). Thus, in a preferred embodiment, the hybrid bidirectional promoter element is selected from the group consisting of a TDH3/ADH1 promoter, a GAL1/GAL10-GPD promoter and a TEF1/PGK1 promoter. In a further preferred embodiment, the hybrid bidirectional promoter element is selected from the group consisting of a TDH3/ADH1 promoter and a GAL1/GAL10-GPD promoter.

In K. marxianus, the sequence of the LAC4-LAC12 promoter may differ between strains. Thus, a hybrid bidirectional promoter element may comprise monodirectional promoter elements from different yeast strains, in particular different strains of K. marxianus. For example, the hybrid bidirectional promoter element may comprise the nucleic acid sequence set forth in SEQ ID NO: 27. This particular hybrid bidirectional promoter element was obtained by fusing the LAC12 proximal part of the LAC4-LAC12 promoter of K. marxianus strain DSM5422 with the LAC4 proximal part of K. marxianus strain NBRC1777.

In a further specific embodiment, the hybrid bidirectional promoter element is a bidirectional GAP1-ADH2 (e.g., KmGAP1-ADH2, SEQ ID NO: 28) promoter (PGAP1-ADH2), which has the advantage that it is not shut down when the recombinant yeast cell enters a stationary phase. This property is highly advantageous for the accumulation of one or more transgene-expressed polypeptides, such as immunogenic polypeptides in a recombinant yeast cell. The GAP1-ADH2 bidirectional promoter element is regulated by the nitrogen source available in the culture medium. The strongest inducing effect has been observed for proline as the nitrogen source.

Thus, in a particularly preferred embodiment, the hybrid bidirectional promoter element comprises a nucleic acid sequence selected from the group consisting of SEQ ID NOS: 27 and 28.

In a further embodiment, the hybrid bidirectional promoter element is composed of two monodirectional promoter elements of different transcriptional strength and/or regulation which is retained upon fusion into a hybrid bidirectional promoter element. Hence, the rate of divergent transcription of the first and the second transgene can be individually regulated giving high/low expression levels for the first or the second transgene or for both transgenes depending on the conditions of cultivation.

If a transgene encodes a gene product that is toxic for the recombinant yeast cell, it is advantageous to separate cell growth and expression of the transgene-encoded polypeptide. For example, a transgene encoding a toxic gene product is placed under the control of an inducible/repressible promoter element and kept repressed in the initial phase of fermentation and is derepressed in the late phase after biomass has accumulated and growth inhibition by the toxic transgene product is irrelevant.

Thus, the hybrid bidirectional promoter element may comprise one inducible or repressible monodirectional promoter element and one constitutive monodirectional promoter element. For example, the inducible or repressible part of the hybrid bidirectional promoter element is selected from the group consisting of a GAL1, GAL10, GAL80, LAC4, LAC12, ADH2, CUP1, DAN1 and MET3/MET17 promoter element. The constitutive part of the hybrid bidirectional promoter element may be selected from the group consisting of a GAP1, GPD1, TEF1, PGK1, ACT1, HXT7, TDH2/TDH3, ADH1, TPI1 and PYK1 promoter element.

A hybrid bidirectional promoter element may also be a synthetic promoter element comprising combined sequence motifs from known promoter elements, wherein the spacer regions are filled with random sequences (Hubmann, Georg; Thevelein, Johan M.; Nevoigt, Elke (2014), Methods in molecular biology (Clifton, N.J.) 1152, S. 17-42).

Promoter activity is typically assessed by its transcriptional efficiency, or expression level. A reporter gene assay can be used, wherein the promoter is fused to a DNA sequence (transgene) encoding a reporter protein that can easily be quantified (e.g., β-galactosidase or Green Fluorescent Protein (GFP)). Reporter protein levels, measured in the form of enzymatic activity in the case of β-galactosidase or fluorescence intensity in the case of fluorescent proteins, such as GFP, serve as readout for promoter activity. More directly, promoter activity can be assessed by quantitation of the (steady-state) RNA level (expression level) of the functionally linked transgene, e.g. via quantitative reverse transcription PCR (qRT-PCR). Since the number of transcription initiation events per time unit can usually not be measured, promoter strength is commonly expressed as RNA level in relation to a given standard RNA with a long halve life. The expression level analysis may be qualitative, quantitative or semi-quantitative, e.g., employing a microarray, Northern blot, RNA sequencing or qRT-PCR. To quantity the gene expression of a given transgene, it is usually sufficient to determine the level of the protein product (e.g., by Western Blot), instead of the mRNA level, because generally the protein level (e. g. level of an immunogenic polypeptide) is the relevant parameter for process optimization.

The bidirectional promoter element may enable similar, or essentially identical expression levels of the first and second transgene it is operably linked to. Alternatively, the bidirectional promoter element may enable different expression levels of the first and second transgene it is operably linked to. Thus, in one embodiment, the bidirectional promoter element enables similar expression levels of the first transgene and the second transgene. In another embodiment, the bidirectional promoter element enables different expression levels of the first transgene and the second transgene.

As used herein, “similar expression levels” means that the expression level of a first transgene deviates from the expression level of a second transgene by 30% or less, 20% or less, 10% or less or 5% or less, preferably by 20% or less. “Different expression levels” means that the expression level of a first transgene deviates from the expression level of a second transgene by at least 50%, at least 60%, at least 70%, at least 80%, at least 90% or at least 100%, preferably by at least 50%. The expression level can be determined by qRT-PCR, Western blot or reporter gene assay as readout, preferably by qRT-PCR or reporter gene assay using a reporter protein. Notably, in Kluyveromyces spp. the endogenous β-galactosidase gene LAC4 interferes with the use of β-galactosidase as reporter protein.

Promoter elements generally fall into several functional classes: constitutive or regulated, i.e., inducible or repressible promoter elements. Repressible promoter elements increase levels of transcription in response to the absence of a repressor, inducible promoters decrease levels of transcription in the absence of an activator or in response to some change in culture conditions, that render the respective activator inactive.

Accordingly, the bidirectional promoter element according to the invention may enable constitutive or regulated expression of the first and second transgene it is operably linked to. Regulated expression refers to expression that is inducible or repressible (e.g., in response to a stimulus), preferably inducible (e.g., by addition of an inducing factor). The sequences regulating the transcription in one and the other direction of the bidirectional promoter element may be the same, or they may be different in sequence, structure and function, such as promoter sequences of different transcriptional activity or strength, e.g. to obtain different transcription or expression levels and a specific transcription or expression ratio of the first and second transgene. Further, the sequences regulating the transcription in one and the other direction of the bidirectional promoter element may be differently regulated with a specific regulatory profile.

In the case of a toxic gene product, encoded by a transgene, it is preferred to initially keep the transgene repressed under the control of a regulated (e.g., inducible) promoter to allow for biomass accumulation and to induce gene expression only at the end of the fermentation by activating or derepressing the promoter. Genes required for cell growth and proliferation are generally under the control of constitutive promoters that are active right from the start of the fermentation.

Thus, in one embodiment, the bidirectional promoter element enables inducible or repressible expression of the first or the second transgene. In a further embodiment, the bidirectional promoter element enables inducible or repressible expression of the first and the second transgene. In a specific embodiment, the bidirectional promoter element enables inducible or repressible expression of the first and the second transgene, wherein the inducer or repressor of promoter activity for the first and the second transgene is different, e.g., a different carbon source (such as galactose instead of glucose), or a different specific chemical substance (e.g., addition of Cu²⁺ ions), antibiotic or by exposure to environmental factor (such as low/high temperature or light intensity). In another specific embodiment, the bidirectional promoter element enables inducible or repressible expression of the first and the second transgene, wherein the inducer or repressor of promoter activity for the first and the second transgene is identical.

In another embodiment, the bidirectional promoter element enables constitutive expression of the first or the second transgene. In a further embodiment, the bidirectional promoter element enables constitutive expression of the first and the second transgene. In yet another embodiment, the bidirectional promoter element enables inducible or repressible expression of the first transgene and constitutive expression of the second transgene, or vice versa.

Some embodiments of the recombinant yeast cell comprise more than one (i.e., at least two) genomically integrated expression cassette. In these embodiments, each genomically integrated expression cassette may comprise identical bidirectional promoter elements, or each genomically integrated expression cassette may comprise a different bidirectional promoter element, as described herein. Alternatively, some of the genomically integrated expression cassettes may comprise identical bidirectional promoter elements and some may comprise different bidirectional promoter elements.

The recombinant yeast cell according to the invention may comprise more than one genomically integrated expression cassette. In a preferred embodiment, the bidirectional promoter element of each expression cassette is different from the bidirectional promoter element of each further expression cassette. This enables individual regulation of expression levels for each of the encoded transgenes, or pair of transgenes.

In a further preferred embodiment, the bidirectional promoter element of each expression cassette is identical to the bidirectional promoter element of each further expression cassette. Alternatively, the inducer or repressor of promoter activity for the bidirectional promoter element of each expression cassette is the same as compared to the inducer or repressor for the bidirectional promoter element of each further expression cassette (e.g., all bidirectional promoter elements are regulated by Gal80-mediated inhibition of the activator Gal4p in the absence of galactose or lactose). In these cases, coordinated, or concerted co-expression of multiple components of a heterodimeric or multimeric protein complex, such as a VLP, in a host cell, enables efficient expression and assembly of such a heterodimeric or multimeric protein complex, e.g., a VLP. This can be in response to an inducing signal, e.g., galactose addition to the medium.

The recombinant yeast cell comprises at least one expression cassette that is stably integrated into the yeast cell genome. The expression cassette may be integrated at various chromosomal loci within the genome of the yeast cell. Particularly useful chromosomal loci for genomic integration of the expression cassette are listed in Table 3. In preferred embodiments, the at least one expression cassette is genomically integrated at a chromosomal locus which is selected such that genomic integration of the expression cassette leads to disruption of a gene coding for a metabolic enzyme rendering the recombinant yeast cell auxotrophic. This enables selection for successful integration of the expression cassettes by selection for restoration of prototrophy (in the form of restoration of the resident gene coding for a metabolic enzyme, or integration of a new prototrophic marker gene).

TABLE 2
Exemplary yeast chromosomal loci for genomic integration of an expression cassette.

| Target | Systematic Name/ | | Selection |
|---|---|---|---|
| Locus | Annotation in Km | Gene Product | Condition |
| ADE1 | KMAR_10170/ADE1 | Phosphoribosylaminoimidazole- | w/o adenine |
| | | succinocarboxamide synthase | |
| ADE2 | KMAR_20566/ADE2 | Phosphoribosylaminoimidazole carboxylase | w/o adenine |
| ADE8 | KMAR_30620/ADE8 | Phosphoribosylglycinamide formyltransferase | w/o adenine |
| ECM31 | KMAR_10771/ECM31 | 3-methyl-2-oxobutanoate hydroxymethyltransferase | w/o pantothenic acid |
| HIS2 | KMAR_10422/HIS2 | Histidinol-phosphatase | w/o histidine |
| HIS3 | KMAR_50033/HIS3 | Imidazoleglycerol-phosphate dehydratase | w/o histidine |
| HIS5 | KMAR_80162/HIS5 | Histidinol-phosphate aminotransferase | w/o histidine |
| LEU1 | KMAR_50143/LEU1 | 3-Isopropylmalate isomerase | w/o leucine |
| LEU2 | KMAR_20461/LEU2 | 3-isopropylmalate dehydrogenase | w/o leucine |
| LYS2 | KMAR_80290/LYS2 | L-aminoadipate-semialdehyde dehydrogenase | w/o lysine |
| LYS5 | MAR_80103/LYS5 | L-aminoadipate-semialdehyde dehydrogenase- | w/o lysine |
| | | phosphopantetheinyl transferase | |
| MET5 | KMAR_40492/MET5 | Sulfite reductase [NADPH] subunit beta | w/o methionine. |
| | | | w/o cysteine |
| MET17 | KMAR_10653/MET17 | O-acetyl homoserine-O-acetyl | w/o methionine. |
| | | serine sulfhydrylase | w/o cysteine |
| TRP1 | KMAR_60134/TRP1 | N-(5′-phosphoribosyl) anthranilate isomerase | w/o tryptophan |
| TRP3 | KMAR_30022/TRP3 | Indole-3-glycerol phosphate synthase | w/o tryptophan |
| TRP4 | KMAR_20433/TRP4 | Anthranilate phosphoribosyltransferase | w/o tryptophan |
| TRP5 | KMAR_20042/TRP5 | Tryptophan synthase | w/o tryptophan |
| TYR1 | KMAR_10786/TYR1 | Prephenate dehydrogenase | w/o tyrosine |
| URA3 | KMAR_10705/URA3 | Orotidine 5′-phosphate decarboxylase | w/o uracil |
| URA5 | KMAR_10567/URA5 | Orotate phosphoribosyltransferase | w/o uracil |

Another group of preferred chromosomal loci for genomic integration of the expression cassette are genes coding for enzymes involved in carbon or nitrogen source metabolism. Examples are listed in Table 4. Disruption or deletion of these chromosomal loci deprives the yeast cell of its ability to utilize specific metabolites as sole sources of carbon or nitrogen (see Table 4).

TABLE 4
Further exemplary yeast chromosomal loci for genomic integration of an expression cassette.

| Gene | Systematic Name/ | | |
|---|---|---|---|
| Name | Annotation in Km | Gene Product | Selection Condition |
| FCY1 | KMAR_80322/FCY1 | Cytosine deaminase | Cytosine as sole nitrogen source |
| GAP1 | KMAR_10360/GAP1 | General amino acid permease | L-citrulline as sole nitrogen |
| | | | source |
| LAC4 + | KMAR_30002/LAC4 + | β-galactosidase and | Lactose as sole carbon source |
| LAC12 | KMAR_30003/LAC12 | lactose permease | |
| XYL1 | KMAR_10659/XYL1 | NAD(P)H-dependent D-xylose | Xylose as sole carbon source |
| | | reductase | |
| XYL2 | KMAR_70036/SOR1 | Xylitol dehydrogenase/Sorbitol | Xylose as sole carbon source |
| | | dehydrogenase 1 | |

Thus, in one embodiment, each expression cassette is genomically integrated at a chromosomal locus selected from the group consisting of ADE1, ADE2, ADE8, ECM31, HIS2, HIS3, HIS5, LEU1, LEU2, LYS2, LYS5, MET5, MET17, TRP1, TRP3, TRP4, TRP5, TYR1, URA3, URA5, FCY1, GAP1, LAC4+LAC12, XYL1 and XYL2. Preferably, each expression cassette is genomically integrated at a chromosomal locus selected from the group consisting of TYR1, ADE2, LEU2, LAC4-LAC12, MET5, HIS3, TRP1, XYL2, and LYS5, more preferably from the group consisting of ADE2, LEU2, MET5 and HIS3.

In case the recombinant yeast cell comprises more than one genomically integrated expression cassette, it is preferred that the expression cassettes are not linked, i.e., they are preferably genomically integrated at chromosomal loci located on different chromosomes. This will assure independent segregation in mitosis thereby offering an easy way to generate different combinations of markers (and as a consequence of different expression cassettes) in the same cell. Accordingly, it is particularly preferred, that the at least two genomically integrated expression cassettes are each genomically integrated at chromosomal loci located on different chromosomes, wherein the chromosomal loci are selected from the group consisting of ADE1, ECM31, HIS2, MET17, TYR1, URA3, URA5, GAP1, XYL1 (chromosome 1); ADE2, LEU2, TRP4, TRP5 (chromosome 2); ADE8, TRP3, LAC4-LAC12 (chromosome 3); MET5 (chromosome 4); HIS3, LEU1 (chromosome 5); TRP1 (chromosome 6), XYL2 (chromosome 7) and HIS5, LYS2, LYS5, FCY1 (chromosome 8). Preferably, the expression cassettes are genomically integrated at chromosomal loci selected from the group consisting of TYR1; ADE2, LEU2; LAC4-LAC12; MET5; HIS3; TRP1, XYL2 and LYS5, more preferably from the group consisting of ADE2, LEU2, MET5 and HIS3.

The genomically integrated expression cassette comprises at least one selection marker. Generally, the term “selection marker” (or selection marker gene) refers to a gene or part (fragment) of a gene that confers a selectable growth phenotype (physical growth characteristics) on a cell receiving that (part of a) gene as, for example through a transformation event. Selection markers generally fall into several types, including positive selectable marker genes which confer a selectable growth phenotype to a cell that receives the marker or negative selectable (counter-selectable) markers that allow only those cells to grow, i.e., to form colonies, that do not comprise the selection marker (e.g., that have lost the selection marker). In the case of negative selectable markers, selection requires a growth medium that inhibits growth of the marker containing host cells. For example, the biosynthetic pathway for the nucleobase uracil is inhibited by the drug 5-fluoro-orotic acid (FOA). This fluorinated orotic acid (orotic acid is a biosynthetic precursor of uracil) is converted into the toxic fluoro-uracil by the enzyme encoded by the URA3 gene. Ura3 mutants are lacking the enzyme and are therefore resistant to 5-FOA and can be selected on plates administered with the drug. The intact URA3 gene is one of the rare examples for a counter-selectable or negative selection marker.

Dominant selection markers, such as genes conferring resistance to an antibiotic or other drug, have the advantage that they can be used for selection in any genetic background sensitive to the drug, including diploid cells. Transformation of an expression cassette as described herein, comprising an antibiotic resistance gene as selection marker, allows the identification of a transformed recombinant yeast cell comprising the expression cassette through the ability of this cell to form detectable colonies on or in the medium comprising the antibiotic. Colony formation requires stable inheritance of the dominant selection marker, which can be achieved by genomic integration. In the absence of a vector that enables inheritance of the marker by autonomous replication in the host cell, colony formation is a safe indicator for genomic integration of the expression cassette.

Some selection markers described herein are metabolic selection markers that enable an otherwise auxotrophic yeast cell to grow, i.e., to form colonies on a selective medium. The selective medium lacks the specific metabolite(s) that is/are essential for the cell, e.g., uracil. For example, the URA3 marker gene can complement a uracil auxotrophy by providing the essential metabolite via the activity of the URA3-encoded enzyme. Alternatively, the selective medium supplies a specific nutrient for selection and colony formation requires that said nutrient is metabolized to provide a component essential for growth, wherein metabolism of the specific nutrient depends on the gene product of the selection marker. For example, lactose can be used as a specific metabolite to select for the enzyme β-galactosidase, which is required to hydrolyse β-galactosides into the respective monosaccharides. Selection using metabolic selection markers may be based on the genomic integration of a gene encoding a metabolic enzyme (the selection marker comprised in the expression cassette), or a fragment thereof, wherein the respective native gene encoding said metabolic enzyme (or a functional equivalent) has been deleted, mutated or disrupted prior to genomic integration of the expression cassette. Said genomic integration of the expression cassette can take place at the chromosomal locus that natively comprises the gene coding for the metabolic enzyme, or at a different chromosomal locus. In other words, genomic integration of the expression cassette can result in reconstitution of a gene coding for a metabolic enzyme at the native chromosomal locus of said gene, or it can lead to introduction of a gene coding for a metabolic enzyme at an alternative locus, wherein the native gene has been rendered non-functional. In the case of genomic integration at the native chromosomal locus, the native gene coding for the metabolic enzyme may have been deleted only partially and the metabolic selection marker may comprise the deleted fragment of said gene coding for the metabolic enzyme, thus reconstituting said gene upon genomic integration of the expression cassette.

The at least one selection marker resides within the expression cassette such that it does not interfere with the function of the bidirectional promoter element, the transgenes or the transcription terminators.

The genes listed as potential chromosomal target loci in Tables 3 and 4 (or fragments thereof) can serve as auxotrophic selection markers. Prior to introduction of the expression cassette at one of these exemplary target loci coding for a metabolic enzyme, the respective locus is mutated, disrupted or deleted, rendering the host cell auxotrophic and thus selectable using the gene coding for the metabolic enzyme (or a fragment thereof) as a positive selection marker.

Further exemplary selection markers are listed in Table 5.

TABLE 5
Exemplary selection marker genes.

| | | | |
|---|---|---|---|
| | Name of | | |
| | resistance | | |
| Marker | marker gene | Gene Product | Selection Condition |

Heterologous genes

| | | | |
|---|---|---|---|
| kanMX | KanR | E. coli transposon Tn 903 kanR gene | resistance to Geneticin (G418) |
| hphMX6 | HygR | Klebsiella pneumoniae | resistance to hygromycin B |
| | | phosphotransferase | |
| natMX6 | NrsR | Streptomyces noursei acetyltransferase | resistance to nourseothricin |
| bleMX6 | BleoR | Tn5 phleomycin-binding protein | resistance to phleomycin |
| cat | CatR | Bacillus subtilis Tn9 acetyltransferase | resistance to chloramphenicol |
| dehH1 | dehH1R | Moraxella sp. Haloacetate | resistance to fluoroacetate |
| | | dehalogenase H-1 | |
| dsdA | dsdAR | E. coli D-serine dehydratase | resistance to D -serine + |
| | | | L-proline |
| mdr3 | mdr3R | Mus musculus P-glycoprotein | resistance to FK520 |
| aroA | aroAR | E. coli 5-enolpyruvylshikimate- | resistance to glyphosate |
| | | 3- phosphate synthase | |
| pat | patR | Streptomyces viridochromogenes | resistance to bialaphos |
| | | Phosphinothricin N-acetyltransferase | |
| R dhfr | R dhfrR | E. coli dihydrofolate reductase | resistance to methotrexate + |
| | | | sulfanilamide |

Endogenous genes

| | | | |
|---|---|---|---|
| CUP1 | CUP1R | S. cerevisiae Metallothionein | resistance to copper and |
| | | | cadmium |
| can1Δ | CAN1R | (S. cerevisiae) arginine permease | resistance to canavanine |

Thus, in one embodiment, the selection marker is a resistance marker or a metabolic selection marker, preferably a metabolic selection marker. In particularly preferred embodiments, the selection marker is selected from the group consisting of ADE1, ADE2, ADE8, ECM31, HIS2, HIS3, HIS5 LEU1, LEU2, LYS2, LYS5, MET5, MET17, TRP1, TRP3, TRP4, TRP5, TYR1, URA3, URA5, FCY1, GAP1, LAC4+LAC12, XYL1, XYL2, kanMX, hphMX6, natMX6, bleMX6, cat, dehH1, dsdA, mdr3, aroA, pat, R dhfr, CAN1 and CUP1. In a particularly advantageous embodiment, the marker gene is selected from the group consisting of ADE2, MET5, HIS3, LEU2, URA3, kanMX, hphMX6 and natMX6.

Advantageously, the selection marker of the genomically integrated expression cassette is coupled to and sandwiched in between a pair of site-specific recombination sequences, optionally between a pair of loxP sites. The selection marker interposing between the paired site-specific recombination sequences is made to be removable from the genomically integrated expression cassettes. These site-specific recombination sequences are special polynucleotide sequences recognizable by a corresponding enzyme or regulatory element to act on. The site-specific recombination sequences can be loxP sites recognizable and capable of reacting with Cre recombinase or other equivalents to carry out recombinase-mediated marker recovery. The Cre/loxP recombination system was developed, originally for S. cerevisiae and later adapted for K. marxianus. Expression of the Cre recombinase from a plasmid results in Cre catalysed excision of DNA sequences between the two loxP sites, e.g., the selection marker gene. Thus, the marker can be used several times while its original site is now marked by a loxP sequence that is left behind by the recombination reaction. For further specific markers, mainly drug resistance markers (kanMX, hphMX6, natMX6) an analogous recycling is possible (Goldstein, A. L.; McCusker, J. H. (1999), Yeast (Chichester, England) 15 (14), S. 1541-1553). The URA3 auxotrophic marker can be recycled by selection for FOA resistance in strains that contain the URA3 marker in knockout cassettes.

In a further preferred embodiment, a specific counter-selection marker may also be included between the paired site-specific recombination sequences to facilitate the marker recovery. Meganuclease recognition sites of I-SceI from the mitochondria of baker's yeast, I-CreI from the chloroplasts of Chlamydomonas reinhardtii and I-DmoI from the archaebacterium Desulfurcoccus mobilis will be of interest to enable marker recovery and counter selection via elimination of DNA elements located between the paired site by site-specific recombination mediated by the specific nuclease. Alternatively, CRISPR/Cas9-mediated counter-selection using guide RNA targeting at the selection marker can also be of interest to enable complete marker recovery.

The genomically integrated expression cassette comprises a first transcription terminator and a second transcription terminator, said first transcription terminator being located immediately downstream of the first transgene and said second transcription terminator being located immediately downstream of the second transgene; wherein the first transcription terminator is operably linked to the first transgene and the second transcription terminator is operably linked to the second transgene. The transcription terminator or terminator sequence signifies and effects end of transcription and release of the transcribed mRNA from the template for peptide or protein translation. In specific embodiments, the first and second transcription terminator are each selected from the group consisting of a TEF1 terminator from Ashbya gossypii (SEQ ID NO: 29), a HIS5 terminator from K. marxianus (SEQ ID NO: 30), a CPS1 terminator from K. marxianus (SEQ ID NO: 31), a CYC1 terminator from S. cerevisiae (SEQ ID NO: 32) and an ADH1 terminator from S. cerevisiae (SEQ ID NO: 33). In a particularly advantageous embodiment, the first and second transcription terminator each are a TEF1 terminator from A. gossypii.

The genomically integrated expression cassette may comprise further transcriptional and translational regulatory sequences, such as enhancer elements. Enhancer elements serve as binding sites for regulatory factors, which may increase promoter activity, when operably linked to a coding sequence. Enhancer elements are cis-acting elements of DNA, usually about from 10 to 300 bp, serving as binding site(s) for an activator which can act on a promoter element to increase the transcription rate. Transcriptional enhancers are commonly orientation and position independent, having been found 5′ and 3′ to the transcription unit, within an intron, as well as within the coding sequence itself.

Expression cassettes for use in eukaryotic host cells (such as yeast cells) usually contain further sequences necessary for the termination of transcription and for stabilizing the mRNA. Such sequences are commonly located 3′ to the translation termination codon, in untranslated regions of polynucleotide sequences coding for eukaryotic or viral polypeptides and contain nucleotide segments transcribed as polyadenylated fragments in the untranslated portion of the mRNA.

The recombinant yeast cell according to the invention may be a member of the Saccharomycesceae family. In one embodiment, the recombinant yeast cell is derived from a genus selected from the group consisting of Andozyma, Ascobotryozyma, Citeromyces; Debaryomyces, Dekkera, Eremothecium, Issatchenkia, Kazachstania, Kluyveromyces, Kodamaea, Lodderomyces, Pachysolen, Pichia, Saccharomyces, Saturnispora, Tetrapisispora, Torulaspora, Williopsis and Zygosaccharomyces. In a preferred embodiment, the recombinant yeast cell is derived from the yeast genus Kluyveromyces. More preferably, the recombinant yeast cell is derived from the yeast species Kluyveromyces marxianus. In a particularly preferred embodiment, the recombinant yeast cell is derived from Kluyveromyces marxianus strain NBRC1777.

The genetic repertoire of S. cerevisiae reveals a specialization in evolution on ethanol fermentation and adaptation to high glucose, fructose or sucrose whereas recombinant yeast cells derived from the yeast genus Kluyveromyces can metabolize a broader spectrum of carbon sources including inexpensive ones, like lactose.

In one embodiment, the recombinant yeast cell is derived from a Crabtree-negative yeast species, preferably, the recombinant yeast cell is Crabtree-negative. Crabtree-positive yeasts, like S. cerevisiae, require more glucose to produce the same number of cells because respiration is glucose-repressed even under aerobic conditions and ethanol is produced at the expense of ATP (adenosine triphosphate). Hence, Crabtree-negative yeasts give a better yield of biomass based relative to the supplied carbon source. Yeast belonging to the genus Kluyveromyces are classified as Crabtree-negative yeasts, which are favoured in industrial fermentation processes. In particular K. marxianus has been observed to be strongly Crabtree-negative, with a Crabtree-negative character that is more pronounced than what could be observed for K. lactis.

K. marxianus has been observed to possess a greater tolerance for elevated temperatures, as compared to many alternative yeast species, which is advantageous in the context of recombinant protein expression, in particular in the context of large-scale production of recombinant yeast cells. Moreover, K. marxianus possesses the highest documented cell division rate of all eukaryotic cell types or organisms, which translates into faster growth of a K. marxianus yeast culture, e.g., in a large-scale production environment.

Generally, cells of yeast exist in a haploid, a diploid or a tetraploid form. The diploid cells may, under appropriate conditions, proliferate for indefinite number of generations in the diploid form. In species with a sexual cycle, diploid cells can also undergo meiosis and sporulate to form haploid cells.

The recombinant yeast cell of the invention may be haploid, diploid, or tetraploid. In one embodiment, the recombinant yeast cell is diploid or tetraploid. The inventors have found that diploid or tetraploid yeast cells are particularly advantageous in that they provide robustness, high rates of growth and protein biosynthesis and thus enable highly efficient expression of the gene products encoded by the first and second transgene.

As used herein, the term “haploid” refers to a cell having a single copy of each chromosome of its normal genomic (chromosomal) complement. The term “diploid” refers to a cell having two copies of each chromosome of its normal genomic complement. The term “allele” designates a specific variant of the gene. Generally, the two parental strains can be distinguished by differences in the DNA sequence between the parental allele (maternally vs paternally-derived or MATa-derived vs. MA Ta-derived). Diploids can be formed by the process of mating between two mating-competent haploid cells. “Mating” of yeast describes a genetically controlled process by which two haploid yeast cells naturally fuse to form one diploid yeast cell with a single nucleus. When two diploid yeast cells naturally fuse, they can form one tetraploid yeast cell with a single nucleus. A yeast cell is “mating competent”, if it is capable of fusing with a further yeast cell by mating or spheroplast fusion. Mating of haploid yeast cells results in diploid yeast cells and sequential mating can further result in tetraploid strains through further mating of the auxotrophic diploids.

In a specific embodiment, the recombinant yeast cell is haploid. Preferably, the haploid recombinant yeast cell is heterothallic, i.e., it has a stable mating type. In other words, it is unable to switch its mating type. Further preferably, the yeast cell comprises deletions and/or mutations rendering a gene required for mating type switching from MATa to MATα or a gene required for mating type switching from MATα to MATa non-functional. The yeast cell may also preferably comprise deletions and/or mutations rendering a gene required for mating type switching from MATa to MATα and a gene required for mating type switching from MATα to MATa non-functional. In a particular embodiment, the yeast cell comprises deletions and/or mutations rendering the KAT1 gene or the ALPHA3 gene non-functional. In a further particular embodiment, the yeast cell comprises deletions and/or mutations rendering the KAT1 gene and the ALPHA3 gene non-functional. Deletions and/or mutations of the KAT1 gene or the ALPHA3 render the recombinant yeast cell heterothallic. Advantageously, the haploid recombinant yeast cell is mating competent. In a particularly preferred embodiment, the recombinant yeast cell is haploid, comprises deletions and/or mutations rendering a gene required for mating type switching from MATa to MATα or a gene required for mating type switching from MATα to MATa (e.g., the KAT1 gene or the ALPHA3 gene) non-functional and is mating competent. In a further particularly preferred embodiment, the recombinant yeast cell is haploid, comprises deletions and/or mutations rendering the genes required for mating type switching from MATa to MATα and mating type switching from MATα to MATa (e.g., the KAT1 gene and the ALPHA3 gene) non-functional and is mating competent.

In a particularly advantageous embodiment, the yeast cell comprises deletions and/or mutations rendering a gene required for mating type switching from MATa to MATα or a gene required for mating type switching from MATa to MATα non-functional, wherein the gene comprising the deletions and/or mutations is linked with a further selection marker, preferably a resistance marker. In another particularly advantageous embodiment, the yeast cell comprises deletions and/or mutations rendering a gene required for mating type switching from MATa to MATα and a gene required for mating type switching from MATα to MATa non-functional, wherein the genes comprising the deletions and/or mutations are each linked with a further selection marker, preferably a resistance marker.

In a highly preferred embodiment, the recombinant yeast cell is haploid, comprises deletions and/or mutations rendering a gene required for mating type switching from MATa to MATα or a gene required for mating type switching from MATα to MATa (e.g., the KAT1 gene or the ALPHA3 gene) non-functional and is mating competent, wherein the gene comprising the deletions and/or mutations is linked with a further selection marker, preferably a resistance marker. In a further highly preferred embodiment, the recombinant yeast cell is haploid, comprises deletions and/or mutations rendering a gene required for mating type switching from MATa to MATα and a gene required for mating type switching from MATα to MATa (e.g., the KAT1 gene and the ALPHA3 gene) non-functional and is mating competent, wherein the genes comprising the deletions and/or mutations are each linked with a further selection marker, preferably a resistance marker.

A selection marker that is linked (i.e., genetically linked, i.e., genomically integrated at a chromosomal locus located on the same chromosome) to a gene required for mating type switching allows for the selection of haploid progeny of a diploid cell that carries a deletion and/or mutation rendering a gene required for mating type switching non-functional.

The invention further relates to a diploid recombinant yeast cell obtained by mating a first and a second haploid recombinant yeast cell as described herein. Generally, diploid yeast cells may be generated by mating a first and a second haploid yeast cell, wherein the first and the second yeast cell are of a different mating type, i.e., the first haploid yeast cell is of mating type MATa and the second haploid yeast cell is of mating type MATα, or vice versa.

Advantageously, diploid recombinant yeast cells that arise from the mating of two heterothallic haploid yeast cells partly, or exclusively, form heterothallic haploid progeny through meiosis and sporulation. In contrast, diploid recombinant yeast cells that result from the mating of two homothallic haploid yeast cells exclusively form homothallic haploid progeny.

Mating competent haploid yeast cells, derived from, e.g., Ascomyceta, including K. marxianus, offer several advantages in the context of the present invention. Mating is a genetic program that enables two haploid recombinant yeast cells of opposite mating types to fuse and generate a diploid recombinant yeast cell with a single nucleus. Mating occurs between cells of the same strain and between cells of different strains but generally requires that strains belong to the same species. The resulting diploid recombinant yeast cell inherits a complete set of chromosomes from each of the haploid parents and mating of two haploid recombinant yeast cells possessing different genetic elements, e.g. different expression cassettes, genetic markers and/or transgenes will give diploid recombinant yeast cells that contain the complete genetic information from both parental haploids. Mating haploid recombinant yeast cells therefore provides a convenient and fast route for recombinant yeast strain construction and consolidation. To select diploid recombinant yeast cells after mating, at least one selectable marker is required in each of the haploid parents, preferably an auxotrophic marker in each haploid recombinant yeast cell. Advantageously, the markers are complementary between the two haploid strains.

Diploid strains have been observed to have greater thermostability as well as a higher tolerance to acid, ethanol, and other fermentation inhibitors than haploid strains. Thus, yeast mating provides an efficient means for recombinant yeast strain construction and diverse possibilities of combining haploid recombinant yeast cells comprising multiple transgenes from a reservoir of haploid recombinant yeast cells, i.e., it is time saving through quick and reliable combination of a variety of parental strains. In addition, the resulting diploid recombinant yeast cells offer superior growth and transgene expression characteristics (e.g., higher possible cell density, higher tolerance with regard to inhibitory compounds, higher yield of transgene-encoded gene product).

However, the diploid recombinant yeast cells of species with a sexual cycle may undergo meiosis and sporulation resulting in, haploid spores that germinate and proliferate, often under conditions of nutritional stresses. Meiosis requires crossing over events between homologous chromosomes, which can lead to loss of genomically integrated sequences (e.g., expression cassettes) present in only one of the two homologous chromosomes. Moreover, meiotic recombination and crossing over events may give haploid progeny that can grow on the diploid selective medium such that a culture of a recombinant production strain contains a mixture of diploids and haploids. Such a situation is unfavourable since it affects reproducibility of the fermentation processes and genetic stability of the production strain. It may also negatively impact the production rate of the gene product encoded by the transgene (e.g., an immunogenic polypeptide). Due to the concern about diploid stability, especially in bioreactor fermentation processes, strategies are necessary to provide genetic stability as well as efficient and reproducible transgene expression in the diploid recombinant yeast cells that are used as production strain. The success of such an approach in bioprocesses is based on careful selection of the integration sites for expression cassettes, a suitable marker strategy and a high genetic similarity between the haploid parent cells.

The enrichment of haploid cells during cultivation of diploids can be prevented by linking each expression cassette to a prototrophic marker gene and by using two haploid recombinant yeast cells that are each auxotrophic and require supplementation of media for growth of the haploid recombinant yeast cells. The tight genetic linkage, i.e. close proximity of transgenes and selection marker means that homologous recombination events that separate the transgene from the marker are extremely rare and can be recognized by genetic screening. Advantageously, the auxotrophic markers are complementary between the two haploid strains (e.g., Ade⁻ and His⁻ or Met⁻ and Leu⁻), such that the diploid recombinant yeast cell will grow in the absence of all supplements (leu, his, met and ade, ura) required by the haploid recombinant yeast cells.

Thus, in a preferred embodiment, the diploid recombinant yeast cell obtained by mating a first and a second haploid recombinant yeast cell as described herein, comprises at least two selection markers genomically integrated at chromosomal loci located on homologous chromosomes. Advantageously, said selection markers are comprised in the genomically integrated expression cassette(s). In a specific embodiment, the first and the second haploid recombinant yeast cell are each auxotrophic and the diploid recombinant yeast cell is able to grow under selection conditions under which the first and the second haploid recombinant yeast cell exhibit reduced or no growth or proliferation. This means that the auxotrophy of the first haploid recombinant yeast cell is (partially) complemented by the genome of the second recombinant yeast cell and vice versa. For example, the first haploid recombinant yeast cell may comprise a (partial) deletion, mutation or disruption of a gene encoding a metabolic enzyme, thus rendering the first haploid recombinant yeast cell auxotrophic. If the second haploid recombinant yeast cell comprises an intact (functional) gene encoding said metabolic enzyme, the resulting diploid recombinant yeast cell is able to grow under selection conditions under which the first haploid recombinant yeast cell exhibits reduced or no growth or proliferation. Preferably, the first and the second haploid recombinant yeast cell are each auxotrophic and the diploid recombinant yeast cell is prototrophic. Further preferably, the selection markers are metabolic selection markers. Further preferably, the chromosomal loci located on homologous chromosomes are the same chromosomal loci on each of the homologous chromosomes (equivalent chromosomal loci of homologous chromosomes).

The diploid recombinant yeast cell may be obtained by mating a first and a second haploid recombinant yeast cell, wherein the first and the second haploid recombinant yeast cell each comprise one, two, three or four genomically integrated expression cassettes, resulting in a diploid recombinant yeast cell comprising two, four, six or eight genomically integrated expression cassettes.

Advantageously, the diploid recombinant yeast cell is obtained by mating a first and a second haploid recombinant yeast cell that comprise different expression cassettes, i.e., expression cassettes comprising different transgenes. Accordingly, in a preferred embodiment of the diploid recombinant yeast cell, generated from a first and a second haploid recombinant yeast cell according to the invention, the first transgene and the second transgene of each expression cassette are different from the first and second transgene of each further expression cassette.

In a specific embodiment, the first and the second haploid recombinant yeast cell each comprise one genomically integrated expression cassette, wherein the genomically integrated expression cassette of the first haploid recombinant yeast cell comprises a first pair of transgenes and the genomically integrated expression cassette of the second haploid recombinant yeast cell comprises a second pair of transgenes, wherein the first and the second pair of transgenes are different. In a further embodiment, the first and the second haploid recombinant yeast cell each comprise two genomically integrated expression cassettes, wherein the genomically integrated expression cassettes of the first haploid recombinant yeast cell comprise a first and a second pair of transgenes and the genomically integrated expression cassettes of the second haploid recombinant yeast cell comprise a third and a fourth pair of transgenes, wherein the first, second, third and fourth pair of transgenes are different. In another embodiment, the first and the second haploid recombinant yeast cell each comprise three genomically integrated expression cassettes, wherein the genomically integrated expression cassettes of the first haploid recombinant yeast cell comprise a first, a second and a third pair of transgenes and the genomically integrated expression cassettes of the second haploid recombinant yeast cell comprise a fourth, a fifth and a sixth pair of transgenes, wherein the first, second, third, fourth, fifth and sixth pair of transgenes are different. In yet another embodiment, the first and the second haploid recombinant yeast cell each comprise four genomically integrated expression cassettes, wherein the genomically integrated expression cassettes of the first haploid recombinant yeast cell comprise a first, a second, a third and a fourth pair of transgenes and the genomically integrated expression cassettes of the second haploid recombinant yeast cell comprise a fifth, a sixth, a seventh and an eighth pair of transgenes, wherein the first, second, third, fourth, fifth, sixth, seventh and eighth pair of transgenes are different.

In another advantageous embodiment, the diploid recombinant yeast cell is obtained by mating a first and a second haploid recombinant yeast cell according to the invention, wherein the first and the second haploid recombinant yeast cell each comprise an identical number of genomically integrated expression cassettes and wherein the expression cassettes are genomically integrated at chromosomal loci located on homologous chromosomes. Preferably, the expression cassettes are genomically integrated at the same chromosomal loci in the first and the second haploid recombinant yeast cell. This results in a diploid recombinant yeast cell comprising pairs of expression cassettes that are genomically integrated at corresponding (homologous) loci in homologous chromosomes.

In one specific embodiment, the diploid recombinant yeast cell is obtained by mating a first and a second haploid recombinant yeast cell, wherein the first and the second haploid recombinant yeast cell each comprise an identical number of genomically integrated expression cassettes and wherein the expression cassettes are genomically integrated at the same chromosomal loci in the first and the second haploid recombinant yeast cell, such that the diploid recombinant yeast cell comprises pairs of expression cassettes that are genomically integrated at corresponding (homologous) loci in homologous chromosomes, which are selected from the group consisting of ADE1, ADE2, ADE8, ECM31, HIS2, HIS3, HIS5, LEU1, LEU2, LYS2, LYS5, MET5, MET17, TRP1, TRP3, TRP4, TRP5, TYR1, URA3, URA5, FCY1, GAP1, LAC4+LAC12, XYL1 and XYL2. Preferably, the chromosomal loci are selected from the group consisting of TYR1, ADE2, LEU2, LAC4-LAC12, MET5, HIS3, TRP1, XYL2, and LYS5, more preferably from the group consisting of ADE2, LEU2, MET5 and HIS3.

The resulting diploid recombinant yeast cell comprises pairs (e.g., one, two, three or four pairs) of expression cassettes that are integrated at corresponding (homologous) chromosomal loci in homologous chromosomes, wherein the corresponding (homologous) chromosomal loci are selected from the group consisting of ADE1, ADE2, ADE8, ECM31, HIS2, HIS3, HIS5, LEU1, LEU2, LYS2, LYS5, MET5, MET17, TRP1, TRP3, TRP4, TRP5, TYR1, URA3, URA5, FCY1, GAP1, LAC4+LAC12, XYL1 and XYL2, preferably from the group consisting of TYR1, ADE2, LEU2, LAC4-LAC12, MET5, HIS3, TRP1, XYL2 and LYS5 and most preferably from the group consisting of ADE2, LEU2, MET5 and HIS3.

Thus, in an exemplary embodiment, the first and the second haploid recombinant yeast cell each comprise a genomically integrated expression cassette at the ADE2 locus. Mating of said first and second haploid recombinant yeast cell results in a diploid recombinant yeast cell comprising a genomically integrated expression cassette at each one of the homologous ADE2 loci. As a result, loss of the genomically integrated expression cassettes due to sister chromatid exchange is suppressed under constant selection pressure for the selection marker genes comprised in each of the genomically integrated expression cassettes. In a further embodiment, the first and the second haploid recombinant yeast cell each comprise two genomically integrated expression cassettes, one at the ADE2 locus and one at the HIS3 locus. In another embodiment, the first and the second haploid recombinant yeast cell each comprise three genomically integrated expression cassettes, one at the ADE2 locus, one at the HIS3 locus and one at the MET5 locus. In yet another embodiment, the first and the second haploid recombinant yeast cell each comprise four genomically integrated expression cassettes, one at the ADE2 locus, one at the HIS3 locus, one at the MET5 locus and one at the LEU2 locus.

Provision of a diploid recombinant yeast cell by mating or spheroplast fusion of a first and a second haploid recombinant yeast cell according to the invention enables efficient generation of diploid recombinant yeast cells comprising a multitude of genomically integrated expression cassettes and thus comprising various different transgenes. In specific embodiments, the transgenes encode immunogenic polypeptides derived from more than one pathogen. For example, the transgenes may encode immunogenic polypeptides derived from more than one strain of the same viral pathogen. Alternatively, the transgenes may encode immunogenic polypeptides derived from different viral pathogens.

In a specific embodiment, the diploid recombinant yeast cell is obtained by mating a first and a second haploid recombinant yeast cell according to the invention, wherein the first and the second haploid recombinant yeast cell each comprise an identical number of genomically integrated expression cassettes and wherein the expression cassettes are genomically integrated at the same chromosomal loci in the first and the second haploid recombinant yeast cell, such that the diploid recombinant yeast cell comprises pairs of expression cassettes that are genomically integrated at corresponding (homologous) loci in homologous chromosomes, wherein each transgene encodes an immunogenic polypeptide derived from a viral pathogen (or an immunogenic fragment thereof), preferably wherein the viral pathogen belongs to the family of Reoviridae, more preferably wherein the viral pathogen is a rotavirus, even more preferably wherein the viral pathogen is Porcine Rotavirus A (PRVA) and most preferably wherein each immunogenic polypeptide comprises a polypeptide selected from the group consisting of Porcine Rotavirus A (PRVA) VP2, VP4, VP6, VP7, NSP2 and NSP4 and one or more immunogenic fragment thereof. For example, each immunogenic polypeptide advantageously comprises an amino acid sequence selected from the group consisting of SEQ ID NO: 1 (PRVA VP2), SEQ ID NO: 2 (PRVA VP4), SEQ ID NO: 3 (PRVA VP6), SEQ ID NO: 4 (PRVA VP7), SEQ ID NO: 5 (PRVA NSP2) and SEQ ID NO: 6 (PRVA NSP4) and one or more immunogenic fragment thereof, preferably from the group consisting of SEQ ID NOs: 1, 2, 3, and 4 and one or more immunogenic fragment thereof.

Advantageously, each immunogenic polypeptide comprises a polypeptide selected from the group consisting of Porcine Rotavirus A (PRVA) VP2, VP4, VP6 and VP7 and immunogenic fragments thereof, wherein the nucleotide sequences encoding the immunogenic polypeptides are set forth in SEQ ID NO: 13 (PRVA VP2), SEQ ID NO: 14 (PRVA VP4), SEQ ID NO: 15 (PRVA VP6) and SEQ ID NO: 16 (PRVA VP7). Each pair of expression cassettes is preferably genomically integrated at corresponding (homologous) chromosomal loci selected from the group consisting of ADE1, ADE2, ADE8, ECM31, HIS2, HIS3, HIS5, LEU1, LEU2, LYS2, LYS5, MET5, MET17, TRP1, TRP3, TRP4, TRP5, TYR1, URA3, URA5, FCY1, GAP1, LAC4+LAC12, XYL1 and XYL2, more preferably from the group consisting of TYR1, ADE2, LEU2, LAC4-LAC12, MET5, HIS3, TRP1, XYL2 and LYS5, most preferably from the group consisting of ADE2, LEU2, MET5 and HIS3.

In a further specific embodiment, the diploid recombinant yeast cell is obtained by mating a first and a second haploid recombinant yeast cell according to the invention, wherein the first and the second haploid recombinant yeast cell each comprise two genomically integrated expression cassettes and wherein the expression cassettes are genomically integrated at the same chromosomal loci in the first and the second haploid recombinant yeast cell, such that the diploid recombinant yeast cell comprises two pairs of expression cassettes (i.e., four expression cassettes) wherein each pair is genomically integrated at corresponding (homologous) loci in homologous chromosomes, wherein the first and second transgene of the first expression cassette comprise the nucleic acid sequence of SEQ ID NO: 13 (PRVA VP2), the first and second transgene of the second expression cassette comprise the nucleic acid sequence of SEQ ID NO: 14 (PRVA VP4), the first and second transgene of the third expression cassette comprise the nucleic acid sequence of SEQ ID NO: 15 (PRVA VP6) and the first and second transgene of the fourth expression cassette comprise the nucleic acid sequence of SEQ ID NO: 16 (PRVA VP7). Preferably, each pair of expression cassettes is genomically integrated at corresponding (homologous) chromosomal loci selected from the group consisting of ADE1, ADE2, ADE8, ECM31, HIS2, HIS3, HIS5 LEU1, LEU2, LYS2, LYS5, MET5, MET17, TRP1, TRP3, TRP4, TRP5, TYR1, URA3, URA5, FCY1, GAP1, LAC4+LAC12, XYL1 and XYL2, more preferably from the group consisting of TYR1, ADE2, LEU2, LAC4-LAC12, MET5, HIS3, TRP1, XYL2 and LYS5, most preferably from the group consisting of ADE2, LEU2, MET5 and HIS3.

In the abovementioned embodiments of the diploid recombinant yeast cell, each pair of expression cassettes at corresponding (homologous) chromosomal loci preferably comprises different selection markers in the first and in the second expression cassette of each pair. Preferably, said different selection markers are each auxotrophic selection markers and the auxotrophic markers are complementary between the first and the second expression cassette of each pair. As a result, the diploid recombinant yeast cell is prototrophic, i.e., it will grow in the absence of all supplements required by the haploid recombinant yeast cells, whereas growth of said haploid recombinant yeast cells (e.g., resulting from sporulation) is suppressed under the same conditions. Moreover, the occurrence of genetic cross-over, which may result in relocation or loss of the expression cassettes, is very low, resulting in a genetically highly stable recombinant yeast strain. It should be noted that, while it is generally desirable to suppress sporulation events and growth of the resulting haploid recombinant yeast cells when using diploid recombinant yeast cells for production purposes (e.g. production of a whole yeast vaccine), sporulation can be advantageous for fast strain construction if new antigen-combinations are of interest (FIG. 14). The possible number of new antigen combinations resulting from meiotic recombination can be calculated by the formula C=(2n)−2 [C=number of new antigen-combinations; n=number of chromosomes with expression cassettes], assuming the expression cassettes are integrated at the corresponding (homologous) chromosomal loci. When mating two haploid yeast cells, each bearing four expression cassettes in chromosomes 1, 2, 3 and 4 (e.g., MATa: Chr.1 TYR1::VP2; Chr. 2ADE2::VP6; Chr. 3LAC4::VP4[P7]; Chr.4 MET5::VP7[G5]/MATa: Chr.1 HIS3::VP4[P6]; Chr. 2::TRP1: VP4[P23]; Chr. 3::XYL2::VP4[P32]; Chr.4: LYS5::VP7[G9]), the resulting diploid yeast cell expresses eight antigens (VP2; VP6; VP4[P7]; VP7[G5]; VP4[P6]; VP4[P23]; VP4[P32]; VP7[G9]). By induced sporulation, 14 new antigenic arrangements can be selected by suitable choice of selection markers, which are comprised in and thus genetically closely linked to the corresponding expression cassettes.

As for each chromosome at least one integration locus is available, the maximum number of new antigenic arrangements (if n=8) is 254. That means 254 new antigenic combinations in the corresponding haploid yeast cells can be selected. The occurring haploids can again be used for mating and subsequent sporulation until a desired antigen combination is created.

By creating a set of haploids covering all known serotypes from a certain virus (e.g. to date, 27 different G- and 37 P-genotypes have been described in both humans and animals for rotaviruses of type A), this method enables fast reaction on new circulating serotype combinations, without time consuming strain construction and genetic verifications (e.g., by PCR, Southern Blot, Western Blot).

The haploid and diploid, preferably, diploid recombinant yeast cells of the invention can advantageously be cultured for production purposes (e.g., production of a whole yeast vaccine). Such production purposes may include growth in minimal media, which media lacks preformed amino acids, and other complex biomolecules, e.g. media comprising a nitrogen source (proline, glutamine or ammonia) and glucose, lactose or galactose as an energy and carbon source and salts as a source of phosphate, calcium and the like. Preferably, such production media lacks selective agents such as antibiotics, amino acids, purines, pyrimidines, etc. Diploid recombinant yeast cells can be grown to high cell density, for example at least about 10 g/L, at least about 25 g/L, at least about 50 g/L, at least about 75 g/L or at least about 100 g/L dry weight.

The invention further relates to a vaccine composition comprising at least one recombinant yeast cell as disclosed hereinabove. The vaccine composition may comprise a haploid, a diploid or a tetraploid recombinant yeast cell. Preferably, the vaccine composition comprises a diploid recombinant yeast cell, most preferably a diploid recombinant yeast cell obtained by mating a first and a second haploid recombinant yeast cell according to the invention.

The amount or dose of the recombinant yeast cell comprised in the vaccine composition according to the invention may vary. Generally, the amount or dose of the recombinant yeast cell comprised in the vaccine composition according to the invention is sufficient to elicit an immune response in a subject, i.e., the vaccine composition comprises an immunogenic amount of at least one recombinant yeast cell as disclosed hereinabove.

In another aspect, the invention relates to the recombinant yeast cell or the vaccine composition as described herein for use in vaccinating a subject against an infectious disease. The invention also relates to a method of vaccinating a subject against an infectious disease, comprising administering the recombinant yeast cell or the vaccine composition as described herein. In a specific embodiment, the subject is a mammal, preferably a pig. In a further specific embodiment, the infectious disease is a viral disease, preferably a viral disease selected from the group consisting of Porcine Rotavirus A or Porcine Rotavirus C, more preferably Porcine Rotavirus A. Thus, in a particularly preferred embodiment, the invention relates to the recombinant yeast cell or the vaccine composition as described herein for use in vaccinating a pig against Porcine Rotavirus A or Porcine Rotavirus C, in particular Porcine Rotavirus A.

In a further particularly preferred embodiment, the invention relates to the recombinant yeast cell or the vaccine composition as described herein for use in vaccinating a pig against Porcine Parvovirus, preferably Porcine Parvovirus 1. In a further particularly preferred embodiment, the invention relates to the recombinant yeast cell or the vaccine composition as described herein for use in vaccinating a pig against African Swine Fever Virus (ASFV).

In a further preferred embodiment, the subject belongs to the subfamily of equinae. For example, the subject is a horse, a zebra, or a donkey. In a particularly preferred embodiment, the invention relates to the recombinant yeast cell or the vaccine composition as described herein for use in vaccinating a subject belonging to the subfamily of equinae against African Horse Sickness Virus, in particular African Horse Sickness Virus serotype 4.

Administration of the recombinant yeast cell or the vaccine composition may be parenteral, enteral, intramuscular, mucosal or oral, optionally in combination with conventional carriers and/or excipients. Preferably, administration is parenteral, most preferably subcutaneous.

The recombinant yeast cell or the vaccine composition may be administered once (e.g., in a one-shot administration scheme), or more than once (e.g., in a prime-boost administration scheme), as appropriate to raise a protective immune response against the pathogen causing the infectious disease. In one embodiment, the recombinant yeast cell or the vaccine composition is administered once. In a further embodiment, the recombinant yeast cell or the vaccine composition is administered at least two times, e.g., two times. In a further embodiment, the recombinant yeast cell or the vaccine composition is administered at least three times, e.g., three times. In a further embodiment, the recombinant yeast cell or the vaccine composition is administered at least four times, e.g., four times. The vaccine composition may comprise at least one physiologically acceptable carrier, diluent, adjuvant and/or excipient. For example, recombinant yeast cells according to the invention may be prepared in a pharmaceutically acceptable carrier, such as in aqueous saline or buffer in a vaccine composition for administration. Such carrier may comprise common pharmaceutic excipients, such as physiologically acceptable salts to adjust osmotic pressure, buffers, preservatives and the like.

Suitable pharmaceutically acceptable carriers are known from, e.g., Remington's Practice of Pharmacy, 13th edition and J. of. Pharmaceutical Science & Technology, Vol. 52, No. 5, September-October, pp. 238-311.

The recombinant yeast cells are inactivated/killed after cultivation and expression of the transgenes and prior to incorporation into the vaccine composition, or prior to use in the method for vaccination. The inactivation can be carried out with any conventionally available method. Particularly suitable for use in the process according to the invention are liquid inactivation (e.g. liquid inactivation for 3 minutes at 68° C.), lyophilization or gamma irradiation (e.g. with 25 or 50 kGy).

As used herein, “vaccinating” or “vaccination” refers to the prevention and/or treatment of a medical condition, such as an infectious disease. The recombinant yeast cell or the vaccine composition are administered to an animal or to a human subject, preferably an animal, in an amount sufficient to elicit an immune response in the subject, preferably, a protective immune response. More preferably, the immune response comprises an immune response that is protective and specific for the immunogenic polypeptide or immunogenic polypeptides that are encoded by each transgene comprised in the at least one genomically integrated expression cassettes.

In a further aspect, the invention relates to a method for the production of a whole yeast vaccine comprising at least one diploid recombinant yeast cell obtained by mating a first and a second haploid recombinant yeast cell according to the invention, the method comprising generation of the diploid recombinant yeast cell by mating of a first and second haploid yeast cell, wherein the first yeast cell is of mating type MATa and comprises a first expression cassette and the second yeast cell is of mating type MATα and comprises a second expression cassette, wherein the first and second expression cassette are stably integrated into the genome of the first and second yeast cell at the same chromosomal locus, wherein the first and second yeast cell are haploid, heterothallic and mating competent; and wherein each transgene encodes an immunogenic polypeptide derived from a pathogen.

In one embodiment of the method for the production of a whole yeast vaccine, the first yeast cell of mating type MATa comprises a third expression cassette and the second yeast cell of mating type MATα comprises a fourth expression cassette, wherein the third and fourth expression cassette are stably integrated into the genome of the first and second yeast cell at the same chromosomal locus. Optionally, the first yeast cell of mating type MATa further comprises a fifth expression cassette and the second yeast cell of mating type MATα further comprises a sixth expression cassette, wherein the fifth and sixth expression cassette are stably integrated into the genome of the first and second yeast cell at the same chromosomal locus. Further optionally, the first yeast cell of mating type MATa further comprises a seventh expression cassette and the second yeast cell of mating type MATα further comprises an eighth expression cassette, wherein the seventh and eighth expression cassette are stably integrated into the genome of the first and second yeast cell at the same chromosomal locus.

In the method for the production of a whole yeast vaccine, the genomically integrated expression cassettes, as well as the transgenes, bidirectional promoter elements, selection marker genes, chromosomal loci, transcription terminators and/or yeast genus, species or strain described hereinabove may be used.

In a preferred embodiment of the method for the production of a whole yeast vaccine, a selection marker gene comprised in the first haploid yeast cell is ADE2 and a selection marker gene comprised in the second haploid yeast cell is URA3, or vice versa.

The cultivation and propagation of the recombinant yeasts according to the invention can be carried out with any conventionally available method. Particularly preferred are processes that lead to high cell yields at low cost. These include fermentation processes, especially processes of high cell density fermentation. Fermentation using a fed-batch fermentation protocol has proven to be particularly advantageous.

In another aspect, the invention relates to a method for the production of a diploid recombinant yeast cell as described herein, from a wild type yeast strain comprising (a) generating a first yeast cell of mating type MATa and a second yeast cell of mating type MATα from the wild type yeast strain, such that the first and second yeast cell are each heterothallic, isogenic and mating competent; (b) transforming each of the first and second yeast cell with at least one deletion cassette, such that the at least one deletion cassette stably integrates into the host cell genome at the same chromosomal locus in the first and in the second yeast cell; such that stable integration into the yeast cell genome results in the disruption of a gene required for the synthesis of an essential compound, preferably wherein the essential compound is an amino acid or a nucleobase; (c) transforming each of the first and second yeast cell obtained in step (b) with at least one expression cassette, such that the at least one expression cassette stably integrates into the yeast cell genome at the chromosomal locus comprising the deletion cassette of step (b), such that the ability of the first and second yeast cell to synthesize the essential compound of step (b) is restored; and (d) generating the diploid yeast cell by mating or spheroplast fusion of the first and second haploid yeast cell obtained in step (c); wherein the wild-type yeast strain comprises no genetic selection marker and wherein each deletion cassette comprises at least one marker gene, preferably an auxotrophic marker gene; and optionally loxP sites flanking the marker gene; and wherein each expression cassette comprises (i) a bidirectional promoter element; (ii) a first transgene and a second transgene, wherein said first and second transgene are located at opposite ends of the bidirectional promoter element and wherein each transgene is operably linked to one side of the bidirectional promoter element; (iii) a first transcription terminator and a second transcription terminator, said first transcription terminator being located immediately downstream of the first transgene and said second transcription terminator being located immediately downstream of the second transgene; wherein the first transcription terminator is operably linked to the first transgene and the second transcription terminator is operably linked to the second transgene; and (iv) at least one selection marker; and (v) a sequence for restoring the gene required for the synthesis of an essential compound of step (b).

An “essential compound” is a compound (e.g., a growth factor or metabolite) that is required for the cell to grow, perform biosynthesis and/or proliferate. The cell may be able to produce all essential compounds, in which case it is prototrophic. Cells that are not able to produce all essential compounds require supplementation of growth media with the essential compound, i.e., said cells are auxotrophic for the essential compound. Essential compounds can be vitamins, amino acids, nucleosides, nucleobases or one or more metabolic precursor thereof. Preferably, the essential compound is an amino acid or a nucleobase or one or more metabolic precursor thereof. For example, the essential compound is selected from the group consisting of adenine, uracil, histidine, leucine, lysine, methionine, cysteine, tryptophan, tyrosine and pantothenic acid.

As used herein, the term “wild-type” refers to a yeast strain that comprises no genetic selection marker. The wild-type yeast strain does not comprise a transgene. In some embodiments, the wild-type yeast strain is a yeast strain that occurs in nature, or that has been isolated from a natural source, i.e., a yeast strain that carries no genetic modification that is not found in the naturally occurring yeast strain.

In a specific embodiment, the wild type yeast strain is derived from a genus selected from the group consisting of Andozyma, Ascobotryozyma, Citeromyces; Debaryomyces, Dekkera, Eremothecium, Issatchenkia, Kazachstania, Kluyveromyces, Kodamaea, Lodderomyces, Pachysolen, Pichia, Saccharomyces, Saturnispora, Tetrapisispora, Torulaspora, Williopsis, and Zygosaccharomyces, preferably from the yeast genus Kluyveromyces. In a particularly preferred embodiment, the wild type yeast strain is derived from the yeast species Kluyveromyces marxianus. Most preferably, the wild type yeast strain is Kluyveromyces marxianus strain NBRC1777.

### EXAMPLES

### Example 1: Generation of a Parental Yeast Strain for Antigen Production

K. marxianus wild strain NBRC1777 was used for the generation of a parental yeast strain for antigen production. K. marxianus wild strain NBRC1777 was originally isolated from the soil in Japan and has been deposited in the NITE Biological Resource Centers (Shibuya City, Tokyo Prefecture, Japan) under the accession number NBRC1777. DNA sequence information for this strain is available under the GenBank accession numbers AP014599 to AP014607 for chromosomes 1 to 8, respectively.

In contrast to the information in the NITE catalog, the K. marxianus wildtype strain NBRC1777 was observed to be homothallic. Most naturally isolated K. marxianus strains are homothallic: i.e., they change their mating type spontaneously by “mating-type switching” to create mixed populations of haploid MATa and MATa, and diploid MATa/MATa cells. A two-component mating-type switching mechanism has been identified in Kluyveromyces lactis, which requires two transposases (Kat1 and Alpha3) for MAT switching. The Alpha3 transposase switches MATa type cells into the MATa type, and Kat1 switches MATa to MATα type. Cernak et al. identified the K. marxianus orthologs of the K. lactis KAT1 and ALPHA3 genes by reciprocal BLASTp searching followed by molecular-genetic experiments (Cernak, Paul; Estrela, Raissa; Poddar, Snigdha; Skerker, Jeffrey M.; Cheng, Ya-Fang; Carlson, Annika K. et al. (2018), mBio 9 (5)).

Using K. marxianus wild strain NBRC1777, a ura3 strain SY3 was isolated by selection towards 5-fluoroorotic acid (5-FOA) resistance (Boeke, J. D.; LaCroute, F.; Fink, G. R. (1984), Molecular & general genetics: MGG 197 (2), p. 345-346). To generate a MATa strain, the KAT1 gene was subsequently inactivated by insertion of a pSY15-based linear M13-F/-R-PCR fragment (FIG. 2-(2)) containing the selectable marker kanMX, flanked by sequences homologous to the chromosomal KAT1 locus. KanR is an antibiotic resistance gene, a dominant marker (Table 5) conferring resistance to the kanamycin derivative G418. Genomic integration results in the formation of G418-resistant yeast colonies. In the cassette, the kanMX marker is flanked by two recognition sites (loxP sites) for the recombinase Cre. By expression of Cre recombinase, the kanMX cassette can be excised very efficiently. One loxP sequence remains as an insertion mutation and the selection for G418 resistance can be used again for genetic modifications at a further genomic locus.

In order to obtain a suitable MATa strain for crossing experiments, the ALPHA3 gene was inactivated in a similar way (FIG. 2-(3)) to obtain strain SY41. ALPHA3 is essential for the mating type change from a to a. After transient expression of the KmKAT1 gene from pSY16 in such a strain (MATa, kat1Δ, a3Δ), clones were obtained that had changed the genotype from MATa to MATa. Due to the successful inactivation of the ALPHA3 gene, the reciprocal change was no longer possible. The resulting strains are SY11 and SY41 (MATa, kat1Δ::loxP-kanMX-loxP, ura3− and MATa, kat1Δ::loxP, ura3−) and SY200 and SY201 (MATa, kat1Δ::loxP, a3Δ::loxP-kanMX-loxP, ura3− and MATa, kat1Δ::loxP, a3Δ::loxP, ura3−). The four strains are isogeneic meaning that they are genetically identical except for their transposases and the resulting (stable) mating type, which is MATa in SY11 and SY41 and MATa in SY200 and SY201. This allows for crossing to generate homozygous diploids. SY41 and SY201 were used as starting strains for the K. marxianus platform provided herein.

The selection marker gene was in each case flanked by direct repeats of the recognition sites (loxP) for the Cre recombinase. The loxP sites were flanked by sequences homologous to the chromosomal target loci. The length of homologous regions depends on the yeast species that is used as a host. Regions of up to 30-50 bp, which can easily be added to the marker cassette by extending the primers in a single PCR amplification step, can be sufficient to obtain site-specific integration of DNA into the chromosome (e.g. S. cerevisiae). In the case of K. marxianus, homologous regions of about 1000 bp are recommended. The cloning of integration cassettes for replacement, disruption or mutational purposes requires fusion of multiple DNA fragments (preferential PCR-based) into a linearized vector. For this purpose, individual PCR-generated fragments with appropriately 15 to 20 bp of complementary base pairs overlapping at their ends were cloned simultaneously into the linearized pUC19 vector using the In-Fusion cloning method according to manufacturer's instructions (Takara Bio USA Inc).

In-Fusion cloning system functions through a ligase-independent mechanism. Firstly, the In-Fusion enzyme (VVpol, Vaccinia Virus DNA-polymerase) creates single-stranded regions at the end of the oligo/PCR insert and linearized vector. This exposes complementary regions on the insert and vector DNA molecules that then spontaneously anneal/fuse through base pairing. Introduction of this annealed/fused DNA into Escherichia coli repairs any single-stranded gaps and results in the synthesis of a recombinant vector containing the oligo/PCR insert (Irwin et al., Vaccinia Virus and Poxvirology: Methods and Protocols, Methods in Molecular Biology 2012, vol. 890). The pUC19 contained in the In-Fusion HD Cloning kit is a commonly used plasmid cloning vector in E. coli that conveys the ampicillin resistance. The molecule is a small double-stranded circle, 2686 base pairs in length, and has a high copy number in E. coli. pUC19 carries a 54 base-pair multiple cloning site polylinker that contains unique sites for 13 different hexanucleotide-specific restriction endonucleases (Yanisch-Perron, C.; Vieira, J.; Messing, J. (1985), Gene 33 (1), S. 103-119). pUC19 cannot replicate autonomously in yeast.

The integration cassette was separated from the bacterial vector using suitable restriction enzymes or PCR amplification. The resulting DNA fragment do not contain bacterial sequences. Transformation of host yeast strains (here K. marxianus) was carried out according to a protocol described in (Nonklang, Sanom; Abdel-Banat, Babiker M. A.; Cha-aim, Kamonchai; Moonjai, Nareerat; Hoshida, Hisashi; Limtong, Savitree et al. (2008), Applied and environmental microbiology 74 (24), S. 7514-7521). Transformants were verified by PCR and Southern blot analyses, respectively.

### Example 2: Genetic Marking of Genomic Sites for Targeted Integration into the K. marxianus Genome

The strains SY41 (MATa, kat1Δ::loxP, ura3−) and SY201 (MATa, kat1Δ::loxP, a3Δ::loxP, ura3−) were transformed with linear fragments containing a selectable marker cassette, as described above.

More gene deletions were performed in iterative cycles of such integrations of various marker genes (prototrophic markers genes (URA3) or drug resistance marker (kanMX, hphMX6, natMX6) successively.

The general procedure for the selection step to test for gene deletions was as follows:
a) Amplification of the cassettes for integrations (i.e., cassettes for replacements, deletions, disruptions or mutations) using pUC19-specific primers (M13-forward/-reverse) in a final volume of 50 μl using Phusion DNA polymerase (Thermo Fisher Scientific).
b) Preparation of a pre-inoculum yeast culture by transferring cells from a YPD plate or frozen stock into YPD medium and incubation by shaking overnight at 30° C. (120 rpm).
c) Inoculation of the main culture by transferring a defined cell count from the overnight culture to reach an optical density at 600 nm (OD 600) of 0.1. Incubate the culture at 30° C. with shaking (120 rpm) until it reaches OD 600 0.8-1.0.
d) Transformation of deletion cassettes into desired host yeast strains.
e) Selection of transformants by plating of cell suspension on selective SD or YPD-drug plates (YPD-G418, YPD-ClonNat, YPD-Hygromycin) and incubation for 48 h at 30° C.
f) Picking of colonies and transfer onto fresh selective SD plates and appropriate YPD-drug plates (YPD-G418, YPD-ClonNat, YPD-Hygromycin) and incubation for 24 h at 30° C. Only colonies that grew on YPD-drug plates were used for further analysis.
g) Pick colonies for colony PCR.
h) Confirmation of the described deletions/disruptions by PCR analysis and subsequent Southern blot analysis.

The following K. marxianus strains were generated by this method.

### ADE2 Locus—Deletion (Ade2Δ::ScURA3):

In K. marxianus, a defined ade24 allele was obtained after transformation of PCR fragments ^{generated with template plasmid pSY}109 and M13-F/M13-R primer pair.

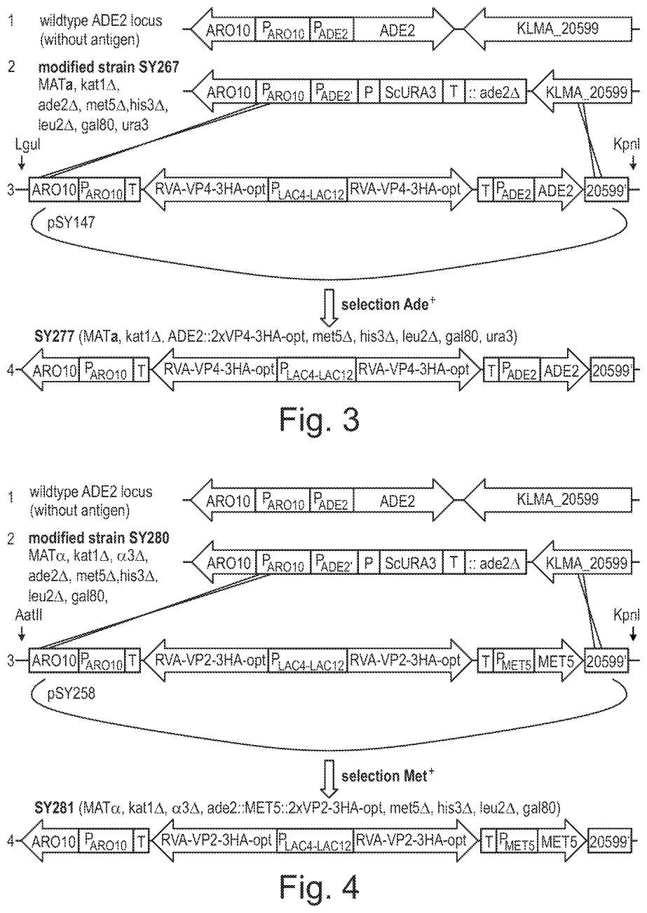

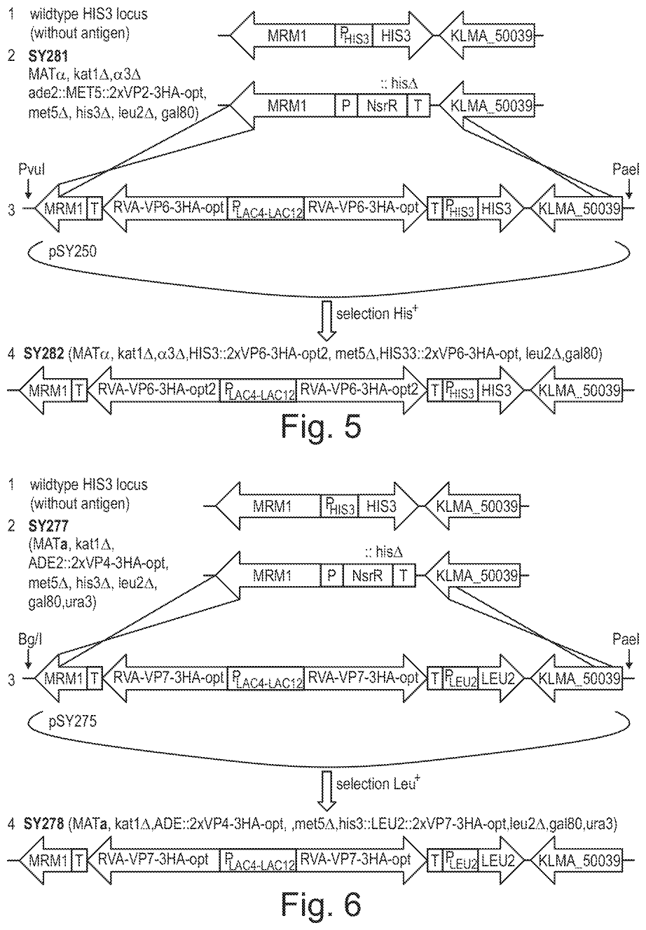

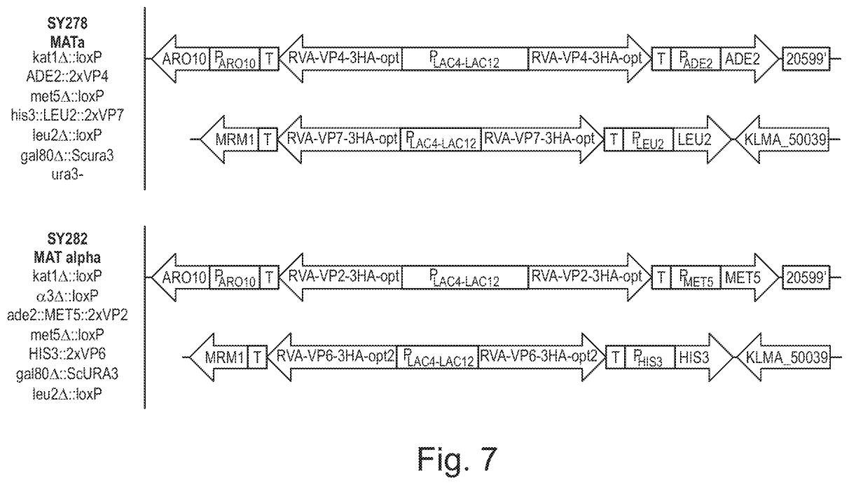

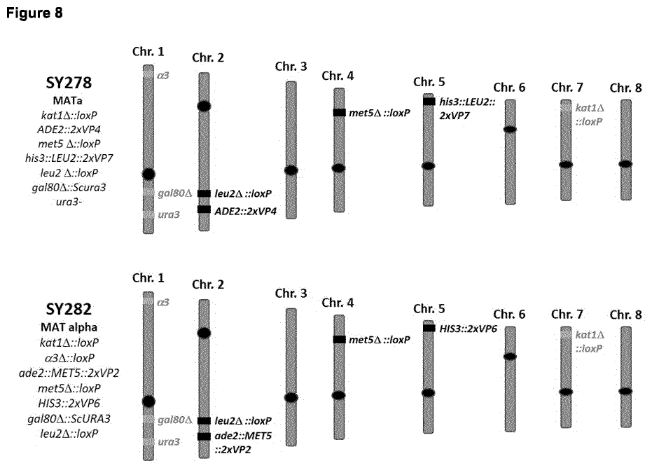

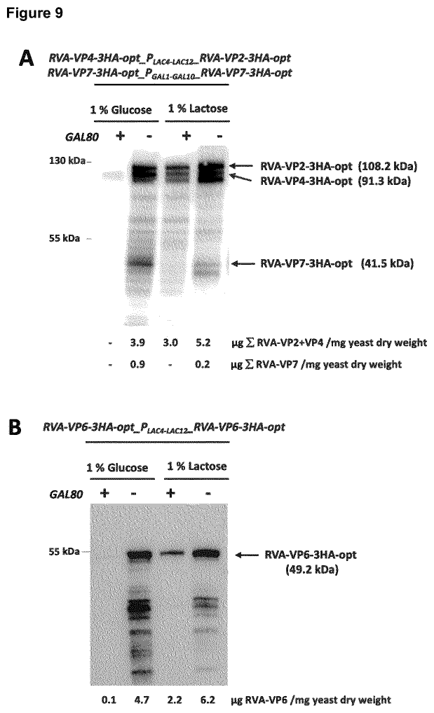

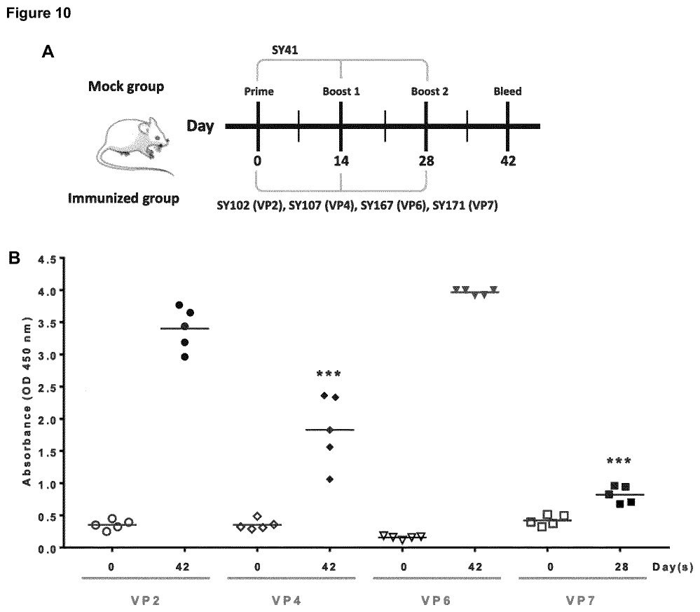

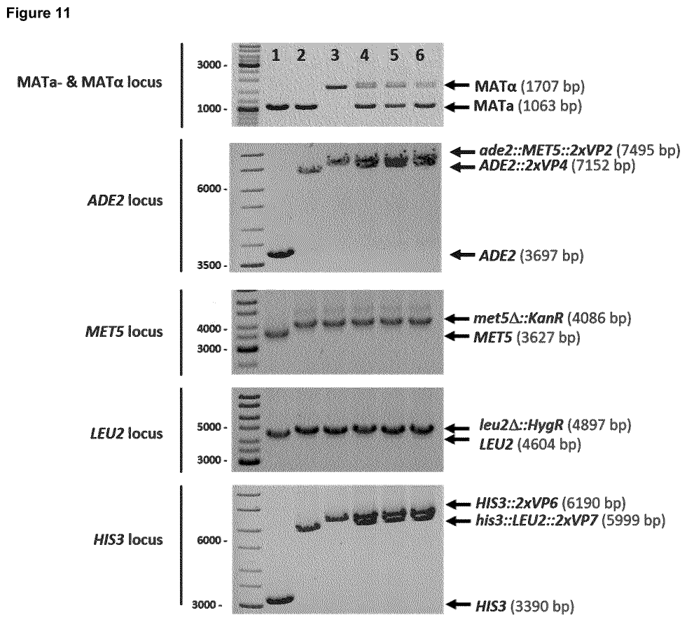

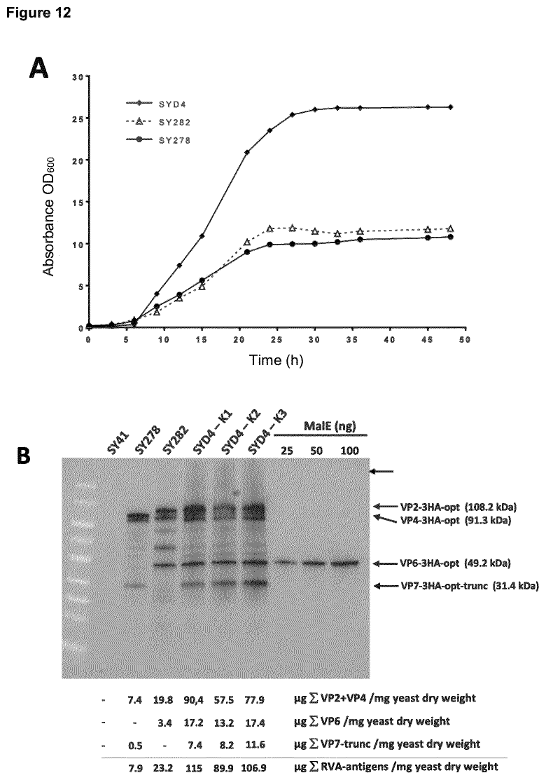

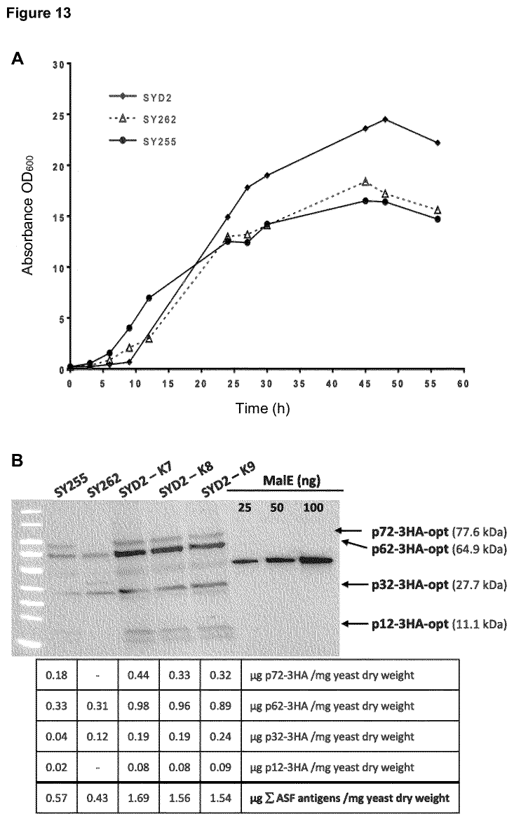

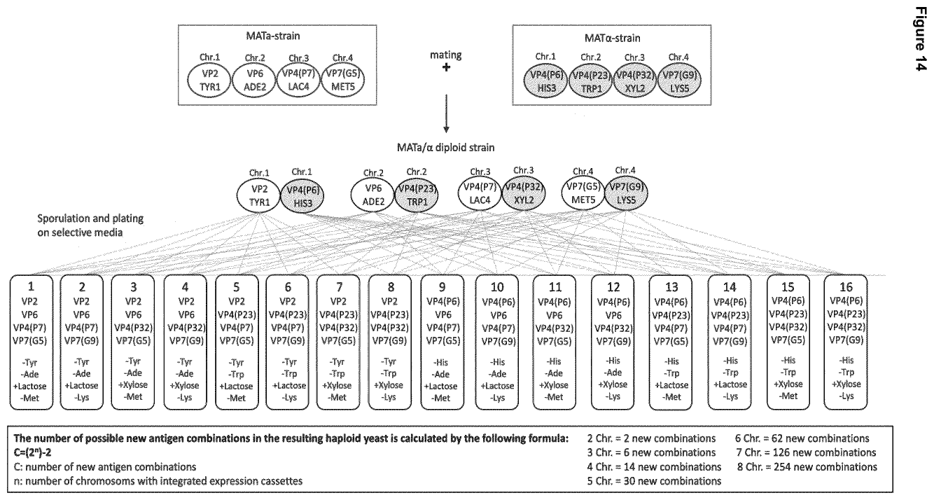

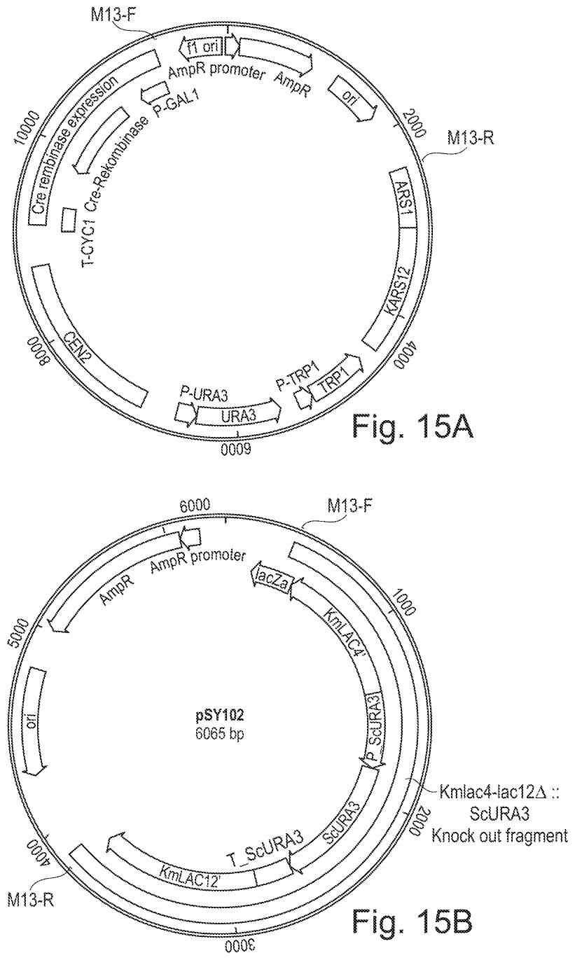

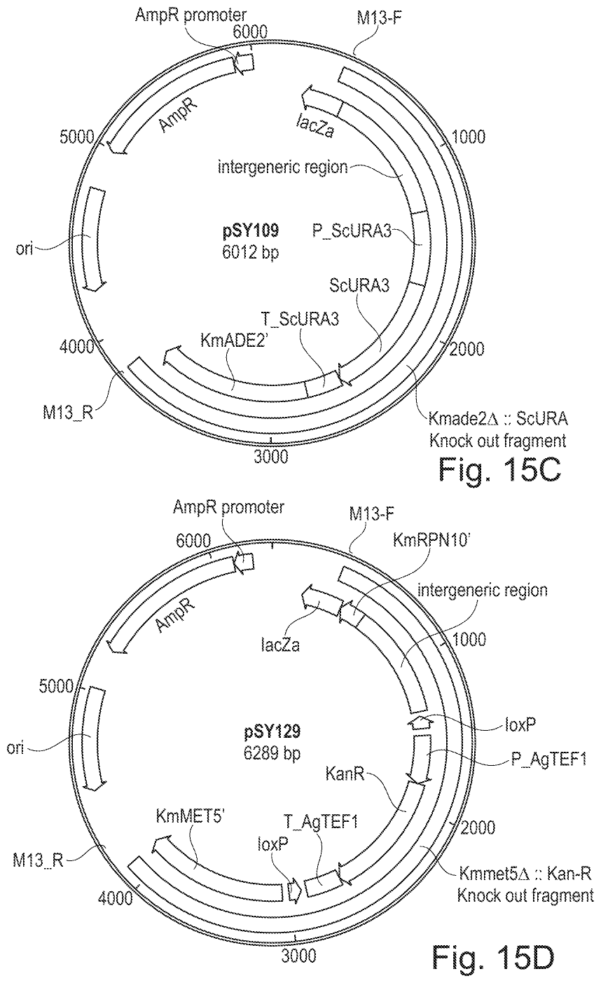

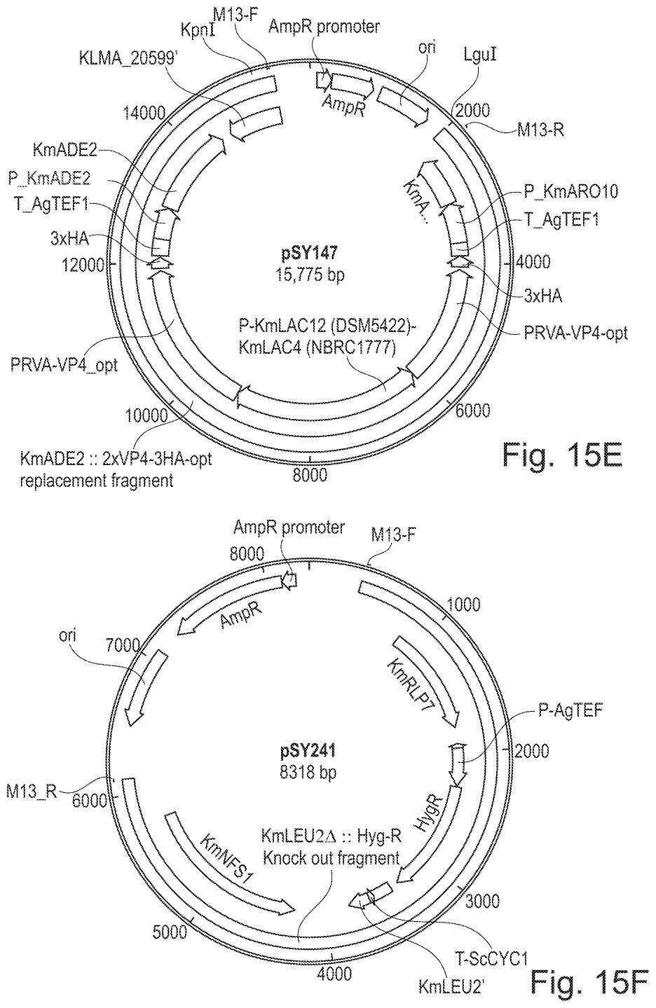

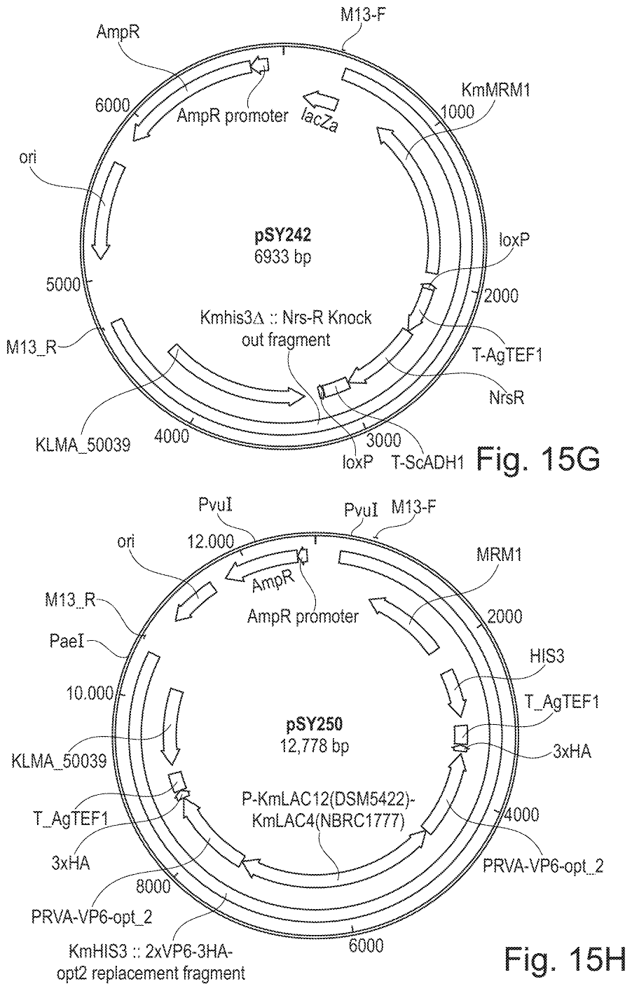

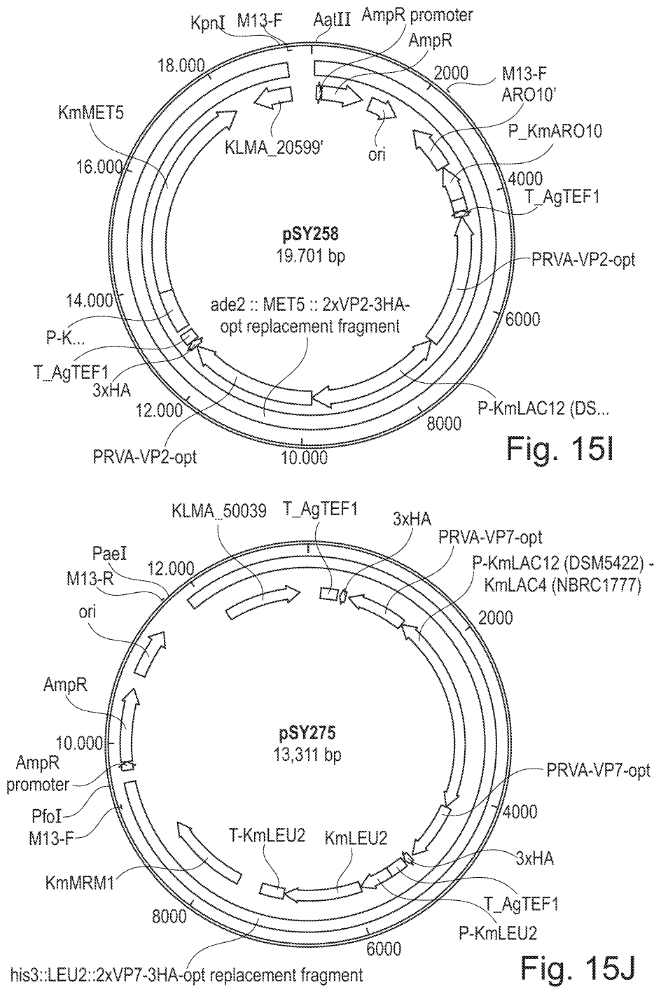

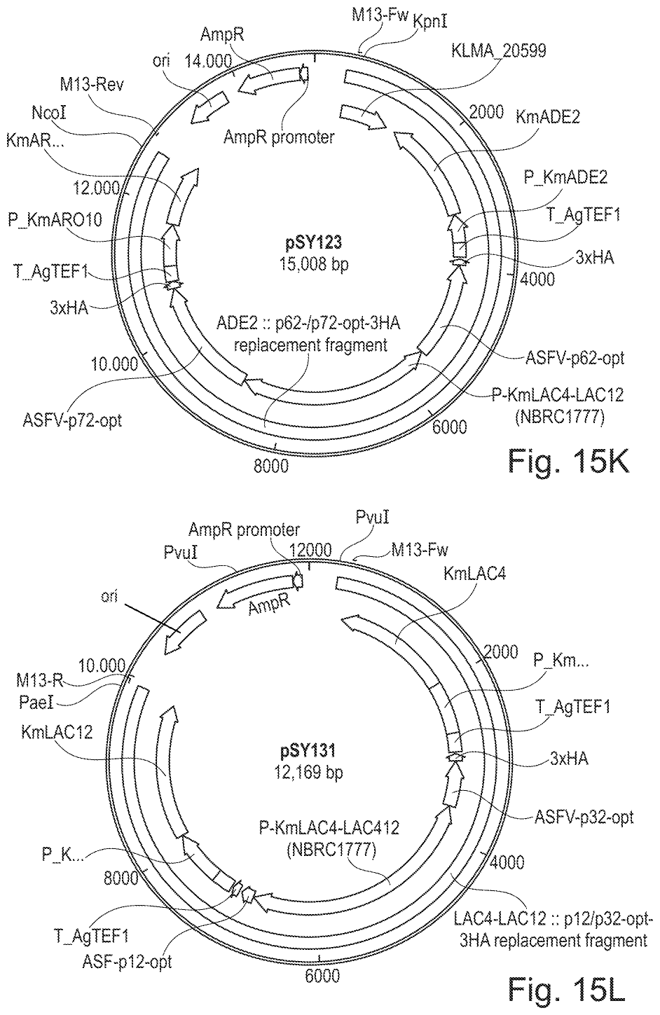

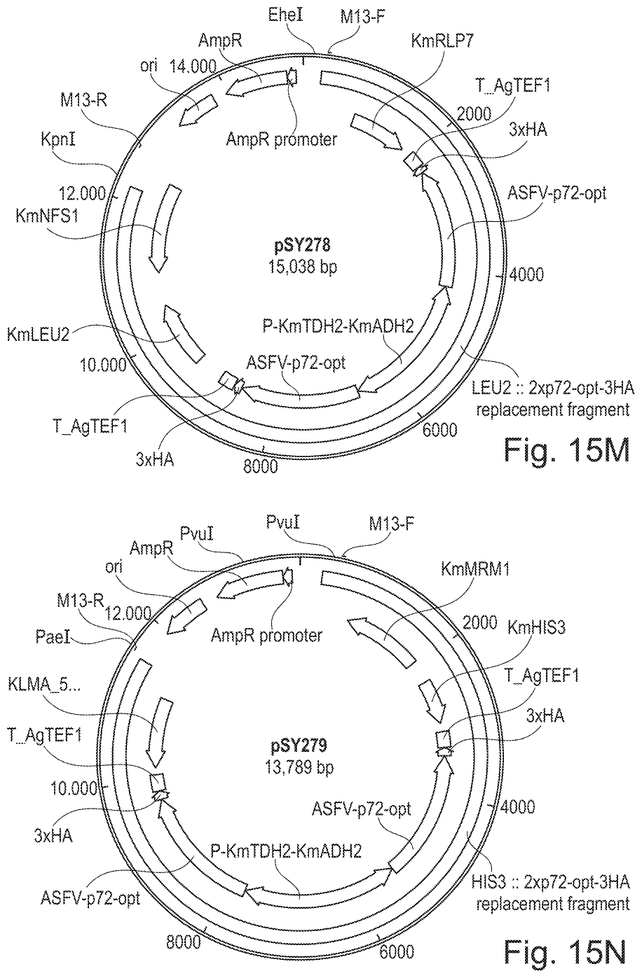

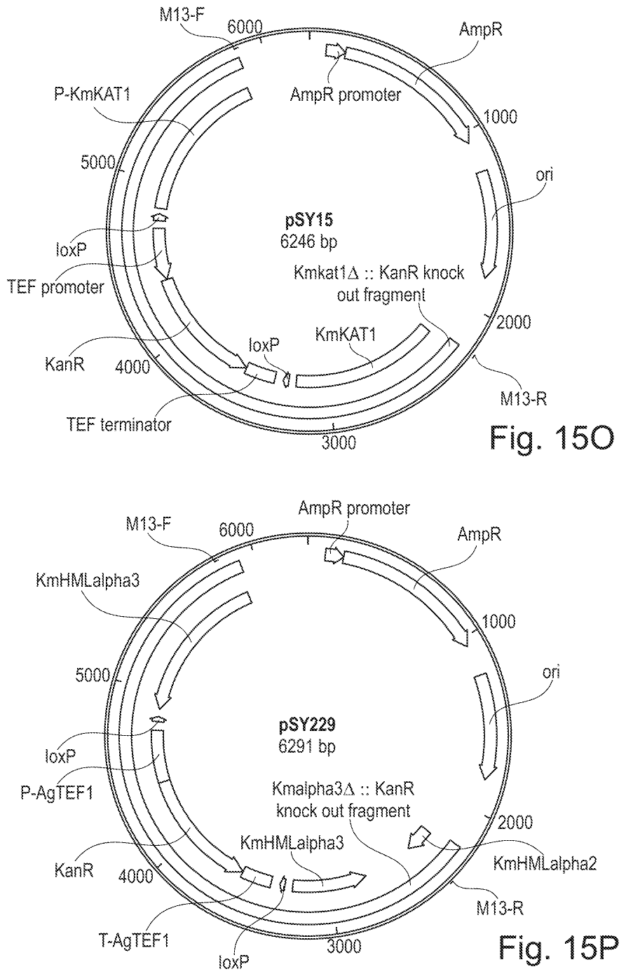

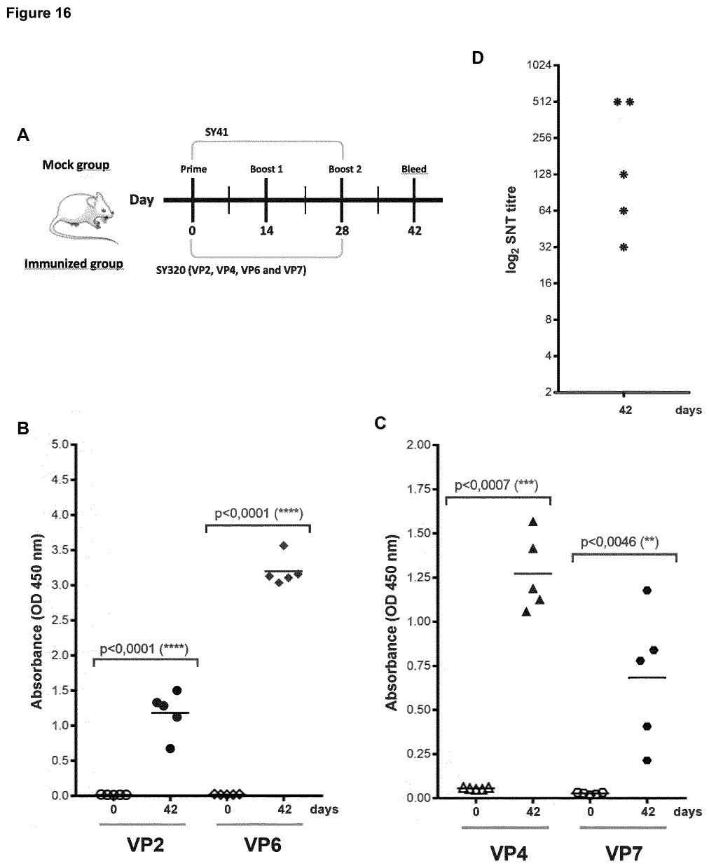

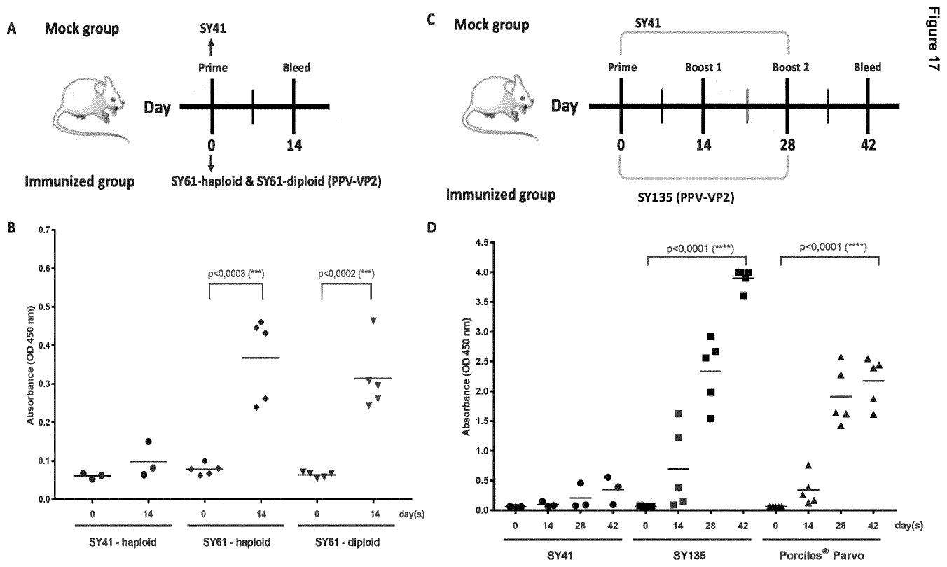

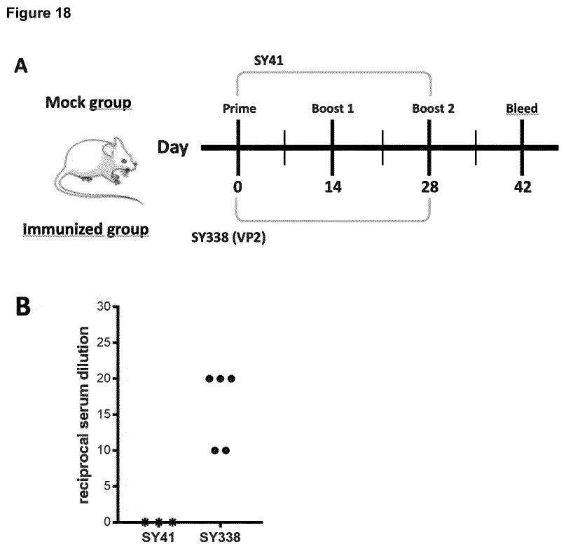

The region 0 bp to +99 bp in the ADE2 locus was replaced by the S. cerevisiae URA3 (SCURA3) marker gene via homologous recombination (FIG. 2). As consequence the ADE2 gene is non-functional. The strains become adenine auxotroph and shows a red coloration of the colonies due to the accumulation of an intermediate of adenine biosynthesis. The ade2A strain can synthesize uracil because the marker in the knockout cassette is the ScURA3 gene. This strain, now marked by the ade2 mutant phenotype, allows the integration of foreign genes at the ADE2 locus. If a suitable integration vector such as pSY147 (FIG. 3) and pSY258 (FIG. 4) is used, the disruption cassette can be replaced by homologous recombination so that an intact ADE2 gene is reconstituted (FIG. 3) or an intact MET5 is integrated (FIG. 4) with loss of the ScURA3 marker gene. This is described in further detail below. The loss of ScURA3 enable a second selection step by 5-FOA counterselection and reduces the effort to identify second site integrations by non-homologous recombinations.

### MET5 Locus—Deletion (Met5Δ::KanR)

In K. marxianus, a defined met54 allele was obtained after transformation of PCR fragments generated with template plasmid pSY129 and M13-F/M13-R primer pair.

The region −883 bp to +299 bp in the MET5 locus was replaced by the E. coli Kanamycin-resistance gene (KanR) via homologous recombination (FIG. 2). As a consequence, the met5A gene is non-functional. The strains become methionine auxotroph but can grow in the presence of the antibiotic G418. Such a met5Δ strain allows the integration of foreign genes at the MET5 locus by selection for methionine growth. If a suitable integration vector is used, the disruption cassette at the met5 locus can be replaced by homologous recombination and MET5 is reconstituted by the loss of the KanR resistance marker gene (as described below).

Furthermore, loxP sites allow recycling of the KanR resistance cassette from the yeast genome by plasmid-based Cre recombinase expression (plasmid pSY16, a pKATUC4-based K. lactis plasmid).

### LEU2 Locus—Deletion (Leu2Δ::HygR)

In K. marxianus, a defined leu2Δ allele was obtained after transformation of PCR fragments generated with template plasmid pSY241 and M13-F/M13-R primer pair.

The region −455 bp to +942 bp in the LEU2 locus was replaced by the E. coli Hygromycin-B resistance gene (HygR) via homologous recombination (FIG. 2). As a consequence, the LEU2 gene is non-functional. The strains become leucine auxotrophic but can grow in the presence of the antibiotic Hygromycin-B. Such a leu2Δ strain allows the integration of foreign genes at the LEU2 locus by selection for leucine growth. If a suitable integration vector is used the disruption cassette can be replaced by homologous recombination so that an intact LEU2 gene is reconstituted with loss of the HygR resistance gene (as described below).

Furthermore, loxP sites allow recycling of the HygR resistance cassette (comprising the hphMX6 marker gene) from the yeast genome by plasmid-based Cre recombinase expression (plasmid PSY16).

### HIS3 Locus—Deletion (His3Δ::NrsR) and Integration of Replacement Cassettes

In K. marxianus, a defined his3Δ null allele was obtained after transformation of PCR fragments generated with template plasmid pSY242 and M13-F/M13-R primer pair.

The region −260 bp to +108 bp in the HIS3 locus was replaced by the Streptomyces noursei Nourseothricin (ClonNAT) resistance gene (NrsR) via homologous recombination (FIG. 2). As a consequence, the HIS3 gene is non-functional. The strains become histidine auxotrophic but can grow in the presence of the antibiotic Nourseothricin. Such a his3A strain allows the integration of foreign genes at the HIS3 locus. If a suitable integration vector such as pSY275 (FIG. 6) and pSY250 (FIG. 5) is used the disruption cassette can be replaced by homologous recombination so that an intact HIS3 gene is reconstituted (FIG. 5) or an intact LEU2 gene is integrated (FIG. 6) with loss of the NrsR marker gene (as described below). Furthermore, loxP sites allow recycling of the NrsR resistance cassette (comprising the NrsR marker gene) from the yeast genome by plasmid-based Cre recombinase expression (plasmid pSY16).

### LAC4-LAC12 Locus—Deletion (Lac4Δ-Lac12Δ::ScURA3)

In K. marxianus, defined lac4Δ-lac12A alleles were obtained after transformation of PCR fragments generated with template plasmid pSY102 and M13-F/M13-R primer pair.

The region +205 bp in LAC4 to +183 bp in LAC12 was replaced by the S. cerevisiae URA3 marker gene via homologous recombination (FIG. 2). As a consequence, the LAC4-LAC12 genes are non-functional and the strain is not able to grow on lactose as sole carbon source but can synthesize uracil due to the integrated ScURA3 gene. Such a lac4-lac12A strain allows the integration of foreign genes at the LAC4-LAC12 locus by selection for lactose growth. If a suitable integration vector is used the disruption cassette can be replaced by homologous recombination so that an intact LAC4 and LAC12 genes, respectively, are reconstituted with loss of the ScURA3 marker gene (as described below).

### Example 3: Codon Optimization of PRVA Antigens and Gene Synthesis

Adaptation of the natural VP2, VP4, VP6 and VP7 genes of the Porcine Rotavirus A strain RVA/Pig-tc/ESP/OSU-C5111/2010/G5P [7] to the codon usage of K. marxianus was analyzed according to (Villada, Juan C.; Brustolini, Otávio José Bernardes; Batista da Silveira, Wendel (2017), DNA research: an international journal for rapid publication of reports on genes and genomes 24 (4), S. 419-434) and using the genomic tRNA database GtRNAdb containing tRNA gene predictions. The translation efficiency of a codon was obtained from the tRNA Adaptation Index (tAI5), where codon relative adaptiveness (w) is the adaptation of a codon to the pool of available cognate tRNAs in the genome which incorporates the different possible tRNA and the wobble pairing rules (Villada et al. 2017).

The VP2, VP4, VP6 and VP7 genes were codon optimized, the encoded protein amino acid sequence of each gene remaining unchanged. Codon optimized VP2, VP4, VP6 and VP7 genes all with linker and C-terminal 3HA epitope were synthesized by GENSCRIPT company, subsequently cloned in pUC19 cloning vector to obtain pUC19-PRVA-VP2-opt, pUC19-PRVA-VP4-opt, pUC19-PRVA-VP6-opt2 and pUC19-PRVA-VP7-opt. Confirmation that the synthetic gene sequence is correct were performed by sequencing.
GenBank accession number of PRVA VP2 gene: KJ450843.1: SEQ ID NO: 7
GenBank accession number of PRVA VP4 gene: KJ450845.1: SEQ ID NO: 8
GenBank accession number of PRVA VP6 gene: KJ450847.1: SEQ ID NO: 9
GenBank accession number of PRVA VP7 gene: KJ450849.1: SEQ ID NO: 10

All used antigenic sequences mentioned are derived from the virus strain Rotavirus A strain RVA/Pig-tc/ESP/OSU-C5111/2010/G5P [7].

### Example 4: Generation of Integration Vectors which Allow for Inducible Antigenic Gene Expression into the Host Genome

All modifications were performed in two steps essentially using a selection/counter-selection strategy. Genetic modifications were conducted in two steps: a selection step, in which the disruption/deletion cassette (e.g. ScURA3, kanMX, natMX6, hphMX6, see FIG. 2) was inserted in the targeted genomic location (as described above), and a special counter-selection step, where the knock out cassette is replaced by an expression cassette resulting in the desired genetic modification (e.g. two antigenic sequences (transgenes) with a bidirectional promoter and transcription terminators).

Integration vectors for expression cassettes were generated as follows. Cloning of expression cassettes for replacements require fusions of multiple DNA fragments (preferential PCR-based) directionally into a linearized vector. For this purpose, independent PCR-generated fragments (for example PCR fragment 1: GOI1-3HA-opt, PCR fragment 2: bidirectional promoter, PCR fragment 3: GOI2-3HA-opt,) with appropriate complementary ends of 15 or 20 bp overlap at their ends were cloned simultaneously into the linearized pUC19-based target vector with homologous sequences to the appropriate target chromosomal loci for homologous recombination using the In-Fusion cloning method according to manufacturer's instructions (Takara Bio USA Inc).

### Example 5: Integration of Expression Cassettes into the Host Chromosome

The general strategy implies that in one of the haploid strains, the mutated gene marking the insertion site is restored by the insertion of the expression cassette comprising the transgenes whereas in the other haploid strains, a new selective marker is introduced as part of the expression cassette linking the old auxotrophic marker gene to a new prototrophic one and both marker genes to the expression cassette containing the antigen coding sequence. This has the advantage that upon crossing of these strains, diploid recombinant yeast cells can be separated from haploids by selection. Since each transgene is tightly linked to the selectable marker gene(s), it is possible to distinguish diploid progeny from haploids by their phenotypes on plates. Since haploids may arise from diploids by meiosis, which is associated with meiotic recombination leading to reciprocal exchanges between homologous chromosomes, it is important for stability of the generated strains to counter-select against meiotic segregants or to find meiotic segregant with new combinations of antigen genes, if desired.

The general procedure of insertion of expression cassettes following counter-selection steps to test for removal of knock out cassettes:
a) Separation of the replacement cassettes from the pUC19 based vector by PCR or digestion using restrictions enzymes, respectively.
b) Transformation of replacement cassettes in desired host yeast strains as described in (Nonklang, Sanom; Abdel-Banat, Babiker M. A.; Cha-aim, Kamonchai; Moonjai, Nareerat; Hoshida, Hisashi; Limtong, Savitree et al. (2008), Applied and environmental microbiology 74 (24), S. 7514-7521).
c) Selection of transformants by plating of cell suspension on selective SD plates and incubation 48 h at 30° C.
d) Picking of colonies and transfer onto fresh selective SD plates and appropriate YPD-drug plates ^{(YPD-G}418, YPD-ClonNat, YPD-Hygromycin) and incubate for 24 h at 30° C. Only colonies that did not grow on YPD-drug plates were used for further analysis.
e) Picking of colonies that grew on SD plates and which were sensitive to the appropriate drug substance for colony PCR.
f) Confirmation of successful integration of expression cassettes by sequencing the PCR products.

The following K. marxianus recombinant yeast cells were generated by this method.

Replacements on ADE2 Locus with Restoration of ADE2 Gene in Strains with MATa-Background

The expression cassette of the pUC19 based vector pSY147 contains consecutively ARO10, ARO10 promoter (P_{ARO10}), TEF1 terminator from Ashbya gossypii (T_{AgTEF1}), PRVA-VP4-3HA-opt (SEQ ID NO: 35), bidirectional promoter (P_{LAC4-LAC12}), PRVA-VP4-3HA-opt (SEQ ID NO: 35), T_{AgTEF1}, ADE2 promoter (P_{ADE2}), ADE2 and the ORF KLMA_20599′ (not characterized). For generation of recombinant K. marxianus strains, the pUC19-based vector is linearized with appropriate restriction enzymes (LguI & KpnI) and transformed into SY267. After successful homologous recombination into the chromosomal ADE2 region of the host genome, the URA3 gene is replaced and the ADE2 gene is restored. The resulting strain (SY277) can synthesize adenine (Ade+), is uracil-auxotrophic and resistant to FOA (ura−, FOA^{R}) and expresses two copies of PRVA-VP4-3HA-opt under control of a bidirectional promoter (P_{LAC4-LAC12}), respectively (FIG. 3).

Replacements on ADE2 Locus with Integration of Intact MET5 Instead of ADE2 in Strains with MATα-Background

The expression cassette of the pUC19 based vector pSY258 contains consecutively ARO10, P_{ARO10}, T_{AgTEF1}, PRVA-VP2-3HA-opt (SEQ ID NO: 34), P_{LAC4-LAC12}, PRVA-VP2-3HA-opt (SEQ ID NO: 34), T_{AgTEF1}, MET5 promoter (P_{MET5}), MET5 and the ORF KLMA20599 (not characterized). For generation of recombinant K. marxianus strains, the pUC19-based vector is linearized with appropriate restriction enzymes (AatII & KpnI) and transformed into SY280. After successful homologous recombination into the chromosomal ADE2 region of the host genome, the URA3 gene is replaced and the MET5 gene is integrated. The resulting strain (SY281) can synthesize methionine (Met+), is uracil-auxotrophic and resistant to FOA (ura−, FOA^{R}) and expresses two copies of PRVA-VP2-3HA-opt under control of a bidirectional promoter (P_{LAC4-LAC12}), respectively (FIG. 4).

Replacements on HIS3 Locus with Restoration of HIS3 in Strains with MATα-Background

The expression cassette of the pUC19 based vector pSY250 contains consecutively MRM1, P_{MRM1}, T_{AgTEF1}, PRVA-VP6-3HA-opt2 (SEQ ID NO: 36), P_{LAC4-LAC12}, PRVA-VP6-3HA-opt2 (SEQ ID NO: 36), T_{AgTEF1}, HIS3 promoter (P_{HIS3}), HIS3, ORF KLMA_50039 (not characterized). For generation of recombinant K. marxianus strains, the pUC19-based vector is linearized with appropriate restriction enzymes (PvuI & PaeI) and transformed into SY281. After successful homologous recombination into the chromosomal HIS3 region of the host genome, the Nourseothricin resistance gene (NrsR) is replaced and the HIS3 gene is integrated. The resulting strain (SY282) can synthesize histidine (His+), is sensitive to Nourseothricin and expresses two copies of PRVA-VP6-3HA-opt2 under control of a bidirectional promoter (P_{LAC4-LAC12}), respectively (FIG. 5).

Replacements on HIS3 Locus with Integration of Intact LEU2 Instead of HIS3 in Strains with MATa-Background

The expression cassette of the pUC19 based vector pSY275 contains consecutively MRM1, P_{MRM1}, T_{AgTEF1}, PRVA-VP7-3HA-opt (SEQ ID NO: 37), P_{LAC4-LAC12}, PRVA-VP7-3HA-opt (SEQ ID NO: 37), T_{AgTEF1}, LEU2 promoter (P_{LEU2}), LEU2, ORF KLMA50039 (not characterized). For generation of recombinant K. marxianus strains, the pUC19-based vector is linearized with appropriate restriction enzymes (BglI & PaeI) and transformed into SY277. After successful homologous recombination into the chromosomal HIS3 region of the host genome, the Nourseothricin resistance gene (NrsR) is replaced and the LEU2 gene is integrated. The resulting strain (SY278) can synthesize leucine (Leu+), is sensitive to Nourseothricin and expresses two copies of PRVA-VP7-3HA-opt whose expression is controlled by one of the bidirectional promoters (P_{LAC4-LAC12}) (FIG. 6).

### Example 6: Optimization of Immunogenic Polypeptide Production by Modification of the GAL80 Locus

The effect of gal80 deletion on transgene expression under the control of P_{LAC4-LAC12} and P_{GAL1-GAL10} was assessed. Different K. marxianus strains expressing PRVA-derived immunogenic polypeptides (VP2, VP4, VP6 and/or VP7) were modified by deletion of the gene coding for repressor protein Gal80. The effect of gal80 deletion in P_{LAC4-LAC12} and P_{GAL1-GAL10} dependent immunogenic polypeptide production was assessed by Western blotting. The corresponding yeast strain with wild-type GAL80 was used as reference strain. Presence (+) or absence (−) of a gal80 deletion are shown above.

Western blots were carried out using 4-20% Mini-PROTEAN TGX Stain free gradient gels (Bio-Rad, CA) loaded with about 0.3 ODE units of freshly harvested yeast cultures. As loading control, gels were activated for Stain free total protein staining by UV exposure for 45 seconds using a Bio-Rad Chemidoc MP imager. Subsequently, proteins were transferred to nitrocellulose membranes (Bio-Rad) using a Transblot Turbo apparatus (Bio-Rad). Membranes were blocked with 5% (w/V) non-fat milk in TBST (Tris-buffered saline (TBS) with tween, 0.05 M Tris, pH 7.4, 0.1 M NaCl, 0.05% (v/v) Tween 20) for 1 hour and then incubated with anti-HA (F-7, sc-7392; 1:3000, Santa Cruz Biotechnology) for 1 hours. Membranes were then washed three times for 10 minutes and then incubated with goat anti-mouse horseradish peroxidase antibody (1:3000, Invitrogen) for 1 h. Membranes were again washed with TBST three times for 10 minutes and incubated with Clarity chemiluminescence substrate (Bio-Rad), imaged on the Chemidoc MP device. Band intensities were quantified by Imagelab 6.0 software (Biorad) and calculated using an internal standard (MalE) taking into account the loaded OD units.

The deletion of gal80 leads to a significant increase in the concentration of immunogenic polypeptide (FIG. 9). The effect is particularly pronounced in glucose containing media.

### Example 7: Immunization of Mice and Serological Tests

The immunogenic potential of recombinant haploid yeast cells as whole yeast vaccines (WYV) was tested. The cells obtained in Examples 5 and 6 were used for subcutaneous application in mice. No abnormalities in weight gain or animal behaviour towards the WYV suspension were observed in previous tolerance studies.

FIG. 10 describe the vaccination in mice with subcutaneously applied, thermal-inactivated, whole yeast cells of the K. marxianus strains generated in Example 5 and 6, i.e., haploid recombinant K. marxianus yeast cells expressing PRVA VP2, VP4, VP6 or VP7 individually.

The immunization study addressed the question, whether the vaccination induce specific antibodies against the applied antigens of the Porcine Rotavirus A.

The immunization experiments were performed with eight weeks old female BALB/C mice (Charles River Laboratories) in groups of five individuals. Per immunization/boost application, 100 μl suspension (containing 1.0 to 5.0 mg of yeast dry weight in physiologic NaCl solution was injected per mouse. During the injection procedure, the mice were anesthetized with 1.5% isoflurane.

Immunization was performed following the schedule which is shown in FIG. 10A. After initial injection, the mice were boosted twice in two weeks intervals (i.e., at day 14 and 28, respectively). Two weeks after the last application, the mice were euthanized with 1.5% isoflurane and blood samples were taken. Mouse serum samples were assayed for the presence of anti-PRVA specific antibodies (VP2, VP4, VP6 and VP7) using an indirect IgG ELISA.

### Indirect ELISA Assay

An indirect IgG ELISA for detection of PRVA-specific antibodies in sera (murine, porcine or other) was developed and evaluated. For this purpose, the expression vector pET15b and the E. coli strain Rosetta(DE3)pLysS (Novagen) were used for the expression of recombinant VP2, VP4, VP6 (expressed as full length proteins of 105.5 kDa, 88.6 kDa and 46.6 kDa, respectively) and VP7 (expressed as aa50Δ N-terminal truncated variant of 33.6 kDa) with C-terminal 6×HIS epitope. Protein expressions and purifications were carried out as described below.

E. coli cultures carrying PRVA expression vectors (pET15b, C-terminal tandem 6×His) were grown at 37° C. until OD600 ˜2. Protein expression was induced using 1.0 mM IPTG, further incubated at 37° C. for 2 h and harvested by centrifugation. The bacteria were resuspended in lysis buffer (50 mM Tris pH 7.8, 300 mM NaCl, 10 mM imidazole, 5% glycerol, 1 mM β-mercaptoethanol, protease inhibitor tablets (Roche), 1/10 (v/v) lysozyme [10 mg/ml]) and incubated for 1 h at 4° C. 1/10 volume of 10% Triton X-100 was added and the lysate was incubated for an additional hour at 4° C. by shaking. Then, 1/15 (v/v) of 5% aqueous protaminsulfate for DNA-precipitation was added and incubated for 20 min at 4° C. The lysate was cleared by centrifugation (15 000 g for 1 h at 4° C.) and the supernatant was incubated with NiNTA resin for 1 h at 4° C. The bound protein was washed once with lysis buffer, once with high salt buffer (50 mM Tris pH 7.8, 1M NaCl, 1, 5% glycerol, 1 mM mM β-mercaptoethanol), once with high imidazole buffer (50 mM Tris pH 7.8, 300 mM NaCl, 40 mM imidazole, 5% glycerol, 1 mM β-mercaptoethanol) und was finally eluted with elution buffer (50 mM Tris pH 7.8, 300 mM NaCl, 250 mM imidazole, 5% glycerol, 1 mM β-mercaptoethanol). The elution fraction was dialyzed overnight in storage buffer (25 mM Tris pH 7.8, 150 mM NaCl, 10% glycerol, 1 mM β-mercaptoethanol).

Polystyrene microtiter plates (Nunc-Immuno™ MicroWell™ 96 well solid plates, Sigma-Aldrich) were coated with 50 ng of recombinant PRVA proteins per well (maximum volume: 400 μl), diluted with 100 μl phosphate buffered saline (pH 7.4) and incubated overnight at 4° C. Plates were washed three times with TBST (25 mM Tris, 0,15 M NaCl, 0.05% Tween-20, pH 7,5, Thermo Fisher Scientific) and then blocked by the addition of 200 μl of blocking buffer per well (Tris-buffered saline pH 7.4 with 0.05% Tween 20, Thermo Fisher Scientific) and incubation at 4° C. for 2 hours. After blocking, the plates were washed three times with TBST and 100 μl aliquots of serum specimens, diluted 1:250 in blocking buffer, were added to the wells. Antigen concentration and serum dilution level for this assay were determined by titration to reach optimal conditions for sensitivity and specificity (data not shown). After 2 hours of incubation at 4° C., the plates were washed three times with TBST. HRP-conjugated (HRP=horseradish peroxidase) goat anti-mouse IgG (Invitrogen) diluted 1:2500 in blocking buffer, were added to the wells in 100 μl aliquots and incubated for 1 h at 4° C. The plates were washed as described above. Binding of specific antibodies was visualized by the addition of 100 μl/well of 1-Step™ Ultra TMB-ELISA Substrate Solution (Thermo Fisher Scientific). After 30 min of incubation at the room temperature, the reaction was stopped by adding 50 μL/well of 1N sulphuric acid and the optical density (OD) was measured at 450 nm (reference filter 540 nm).

Two weeks after the last subcutaneous application, the mice sera were analyzed for the presence of anti-VP2, -VP4, -VP6 and -VP7 antibodies, respectively, with an indirect IgG ELISA that was established for this study. In comparison to animals that were treated with non-PRVA expressing K. marxianus strain SY41, all vaccine strains showed significantly increased levels of PRVA specific antibodies anti-VP2, -VP4, -VP6 and -VP7, respectively.

PRVA-positive pig sera served as a positive control. The zero sera before the vaccination or a non-specific antibody were used as a negative control. The results of the ELISAs are shown in FIG. 10.

Using the recombinant haploid yeast cells of Example 5 and 6, successful immunization against all four transgene-expressed PRVA antigens could be achieved.

### Example 8: Generation of Diploid Recombinant Yeast Cells Expressing PRVA Antigens by Mating

Diploid recombinant yeast cells comprising multiple transgenes were obtained by mating of previously generated haploid recombinant yeast cells. The auxotrophic strains used for this example are the SY278 (MATa, kat1Δ, ADE2::2×VP4-3HA-opt, met5Δ, his3::LEU2::2×VP7-3HA-opt, leu24, gal80, ura3) and SY282 (MATa, kat1Δ, a3Δ, ade2::MET5: 2×VP2-3HA-opt, met5Δ, HIS33::2×VP6-3HA-opt, leu2Δ, gal80) (FIGS. 7 and 8), which require supplementation with methionine, histidine and uracil or adenine and leucine, respectively, for growth.

In principle, any two complementing sets of auxotrophic strains resulting from genomic integration at chromosomal loci selected from Tables 3 and 4 could be used for the construction and maintenance of diploid strains. The haploid recombinant yeast cells used in this example are especially suited for this method for two reasons. Firstly, they grow more slowly than diploid strains that are the result of their mating or fusion. Thus, if a small number of haploid cells arise through meiosis or other mechanism, the diploid strain will outgrow them in culture. Secondly, it is easy to monitor the ploidy state by striking out the culture on plates containing all auxotrophic markers (LEU, MET, HIS, ADE, URA) vs. plates without any of these supplements. A different number of colonies comparing both plate types allow a percentage calculation of haploids present in the culture.

The diploid strains were constructed by mating the haploid strains, followed by selection on plates without any amino acids or bases. Since both expression cassettes had been integrated into the ADE2 and HIS3 locus, haploid progeny resulting from diploid sporulation could only contain two of the four expression cassettes that were genomically integrated at homologous chromosomal loci. Consequently, only diploid cells comprising all five prototrophic genes (ADE2, MET5, HIS3, LEU2, URA3) were able to grow without metabolite supplementation and capable of simultaneously expressing all immunogenic polypeptides, PRVA-VP2, -VP4, VP6 and VP7.

The general procedure for mating of haploid parental strains according the description in (Kutyna, Dariusz R.; Cordente, Antonio G.; Varela, Cristian (2014), Methods in molecular biology (Clifton, N.J.) 1152, S. 157-168):
a) Inoculation of two haploid strains from a YPD plate or frozen stocks into YPD medium (5 mL) and incubation overnight at 30° C. and shaking (120 rpm).
b) Transfer 100 μL of each (haploid) culture into the same sterile 1.5-2.0 mL microcentrifuge tube and mixing thoroughly by vortexing or pipetting.
c) Transfer of mixed culture onto a fresh selective SD plate, air-drying, and incubation at 30° C. During this incubation period, cells of opposite mating types fuse to create diploids.
d) Pick single colonies and plate out several serial dilutions of mated mixture selective SD plates and incubate for at 30° C. until single colonies are fully developed.
e) Verify ploidy of cells by using mating type PCR primers.

Formation of diploids was confirmed by colony PCR (cPCR) using a primer set specific to the MAT loci (MATa and MATα, respectively), as well as the four antigenic sequences integrated at the ADE2 and HIS3 loci (FIG. 11). Primer sequences are listed in Table 6. A stable diploid population was observed in the strain SYD4.

TABLE 6
Primers used for PCR-based ploidy analysis.

| | Primer | | Primer | Product |
|---|---|---|---|---|
| Reaction | Name | Primer Sequence (5′ to 3′) | Description | size |
| MATa | P1 | CAAATGTTGTGGCTGCACCTAC | SLA2-F | 1063 bp |
| | P2 | CAAACGTATGCGTACGAAATC | MATa-R | |
| MATα | P1 | CAAATGTTGTGGCTGCACCTAC | SLA2-F | 1707 bp |
| | P3 | CTCAGGATATAGGAAACATGAAGG | MATalpha-R | |
| ADE2-WT | P200 | AAGAAGACTTAGCCATGGCA | KmARO10-F | 3697 bp |
| | P204 | GAAGAACCAATAATGTTAATATGAC | KmADE2-R | |
| ade2Δ:: | P200 | AAGAAGACTTAGCCATGGCA | KmARO10-F | 4451 bp |
| SCURA3/ | | | | |
| ade2Δ:: | P204 | GAAGAACCAATAATGTTAATATGAC | KmADE2-R | |
| Scura3 | | | | |
| ADE2:: | P200 | AAGAAGACTTAGCCATGGCA | KmARO10-F | 7152 bp |
| 2xVP4 | P162 | TATGAAAACTGATTATCGTCCTGT | P-LAC4-LAC12 | |
| ade2:: | P200 | AAGAAGACTTAGCCATGGCA | KmARO10-F | 7495 bp |
| MET5:: | P162 | TATGAAAACTGATTATCGTCCTGT | P-LAC4-LAC12 | |
| 2xVP2 | | | | |
| HIS3-WT | P532 | TATGTCTGCTTTGGAGCACA | KmMRM1-F | 3390 bp |
| | P512 | GTTGACGTTAACATGATTGGA | KmAGP1-R | |
| his3Δ:: | P532 | TATGTCTGCTTTGGAGCACA | KmMRM1-F | 3547 bp |
| NrsR | P512 | GTTGACGTTAACATGATTGGA | KmAGP1-R | |
| HIS3:: | P118 | ATCTTTCAGTTCTCGATGAG | P-LAC4-LAC12 | 6190 bp |
| 2xVP6 | P512 | GTTGACGTTAACATGATTGGA | KmAGP1-R | |
| his3:: | P118 | ATCTTTCAGTTCTCGATGAG | P-LAC4-LAC12 | 5999 bp |
| LEU2:: | P512 | GTTGACGTTAACATGATTGGA | KmAGP1-R | |
| 2xVP7 | | | | |
| MET5-WT | P375 | CGGCAAAAAAACGCAGGATCTG | Km-P-RPN10-F | 3627 bp |
| | P172 | ATTGAGAATCACCTAAACCAAACACGG | KmMET5-R | |
| met5Δ:: | P375 | CGGCAAAAAAACGCAGGATCTG | Km-P-RPN10-F | 4086 bp |
| KanR | P172 | ATTGAGAATCACCTAAACCAAACACGG | KmMET5-R | |
| LEU2-WT | P533 | ACTGCAGTTAAGATTCCAGC | KmRPL7-F | 4604 bp |
| | P514 | GCCAGAAATACAACACCTCT | Km-T-NFS1 | |
| leu2Δ:: | P533 | ACTGCAGTTAAGATTCCAGC | KmRPL7-F | 4897 bp |
| HygR | P514 | GCCAGAAATACAACACCTCT | Km-T-NFS1 | |

The diploid strains were examined for growth and antigen expression by Western blot analysis. Western blots were carried out as described above.

Growth behaviour and recombinant protein expression patterns were compared between VP2, VP4, VP6 and VP7 expressing haploid K. marxianus strains and their isogenic diploid counterpart. The diploid K. marxianus offers a higher protein production compared to its haploid counterparts (FIG. 12). Therefore, the use of diploid strains as hosts for recombinant protein expression (such as immunogenic polypeptides) results in an increased yield of recombinant protein per dry yeast weight or culture volume.

Diploid recombinant yeast strain stability analysis: The percentage of diploid cells in the population was determined by plating on selective media and followed for 48 h of cultivation (in shake flask culture). Several hundred colonies were analyzed by colony PCR, revealing that more than 98% of colonies growing on selection media were diploid (data not shown). As controls, parental haploid strains carrying either leu- and ade- or met- and his-auxotrophies did not form any colonies on selection plates missing all four nutrients (amino acids or nucleobase, respectively). Therefore, despite of occasional sporulation events during fermentation, it was observed that the majority of the yeast population was maintained in diploid state.

### Example 9: Generation of Diploid Recombinant Yeast Cells Expressing ASFV Antigens by Mating

A further diploid recombinant yeast strain expressing African Swine Fever Virus (ASFV) proteins p12, p32, p62 and p72, each with 3HA epitope tag, was generated as follows.

The full-length open reading frame (ORF) of codon optimized genes O61R (coding for the protein p12); CP204L (coding for p32); CP530R (coding for p62) and B646L (coding for p72) were synthesized based on ASFV isolate Georgia/2007 (GeneBank: FR682468.1) and cloned into the common plasmid vector pUC19 (GENESCRIPT). Confirmation of the synthetic gene sequence was performed by sequencing.

Integration vectors which allow antigenic gene expression into the host genome were generated using the In-Fusion cloning method as described above (Example 4).

For generation of recombinant strains, the pUC19-based vectors (pSY123, pSY131, pSY278, pSY279) were linearized with appropriate restriction enzymes and step-wise transformed in haploid K. marxianus strains (MATa or MATa). Integration into the host genome occurred by homologous recombination into the chromosomal LAC4-LAC12, ADE2, LEU2 region (SY255, MATα) or LAC4-LAC12, ADE2, HIS3 regions (SY262, MATa) (Table 7, FIG. 13). Each resulting strain expresses six copies of ASFV genes whose expression is controlled by one of the bidirectional promoters (P_{LAC4-LAC12} Or P_{GAP1-ADH2}). Integrity and correct integrations of the recombinant genes was confirmed by PCR, Southern Blot as well as Western Blot analysis.

TABLE 7
Haploid and diploid recombinant yeast cells used in this example

| Strain | MAT | Integrations | ASFV antigens |
|---|---|---|---|
| SY255 | haploid; MATα | LAC4-LAC12::p12 + p32, | 1x p12, 1x p32, 1x |
| | | ADE2::p62 + p72, LEU2::2xp72, | p62, 3x p72 |
| | | his3Δ::NrsR, gal80Δ::Scura3, ura3- | |
| SY262 | haploid; MATa | LAC4-LAC12::p12 + p32, | 1x p12, 1x p32, 1x |
| | | ADE2::p62 + p72, HIS3::2xp72, | p62, 3x p72 |
| | | leu2Δ::HygR, gal80Δ::ScURA3 | |
| SYD2 | diploid; | mating of SY255 and SY262 | 2x p12, 2x p32, 2x |
| | MATα/MATa | | p62, 6x p72 |

Optimization of immunogenic polypeptide production was achieved by deletions of the GAL80 locus.

The diploid recombinant yeast cells SYD2 comprising multiple transgenes were obtained by mating of previously generated haploid recombinant yeast cells followed by selection on plates without any amino acids or bases, i.e., selection for prototrophic strains. Conformation of diploids was accomplished by colony PCR (cPCR) using a primer set specific to the MAT loci (MATa and MATα, respectively), as well as the four antigenic sequences integrated at the LAC4-LAC12, ADE2, LEU2 and HIS3 loci. A stable diploid population was observed in the strain SYD2. Subsequently, comparison of growth and recombinant protein production between haploid strains (SY255, SY262) and their isogenic diploid strain SYD2 obtained by mating were determined (FIG. 13).

### Example 10: Immunization of Mice with K. marxianus Strain Expressing Four PRVA Antigens VP2, VP4, VP6 and VP7

The K. marxianus strain used for the immunization in this Example was produced according to the general procedure described in Examples 5 and 6.

TABLE 8
Recombinant yeast cells used in this example

| Strain | MAT | Integrations | PRVA antigens |
|---|---|---|---|
| SY320 | haploid; MATa | MET5: 2xVP2 | 2x VP2, 2x VP4, 2x |
| | | HIS3: 2xVP4 | VP6, 1x VP7, |
| | | ADE2: 2xVP6 | 1xNSP4 |
| | | LEU2: 1xVP7Δ50 + | |
| | | 1xNSP4 | |
| | | gal80Δ::ScURA3, | |
| | | ura3- | |
| SY41 | haploid; MATa | ura3- | — |

VP7Δ50 is a 50 amino acid N-terminally truncated variant of full-length VP7, lacking it's import signal peptide for the endoplasmic reticulum and membrane anchor. As a consequence, the protein is restrained in the cytosol.

Eight weeks old female BALB/c mice were vaccinated subcutaneously three times with SY320 (VP2, VP4, VP6 and VP7) at day 0, 14 and 28 with 2 mg yeast dry weight (FIG. 16A). Control animals received a wildtype strain SY41 without antigen. Sera were collected 14 days after each vaccination for measuring seroconversion.

Mouse sera were analyzed for the presence of PRVA-antigen-specific antibody against VP2 and VP6 (dilutions of sera 1:500) (FIG. 16 B) and VP4 and VP7 (dilutions of sera 1:100) (FIG. 16 C) by an IgG ELISA assay. The individual points represent individual absorbance at 450 nm. Data are representative of at least two independent experiments with similar results and average SEM value of five mice. The positive control for the ELISA was a serum of a PRVA infected pig, as negative control either zero sera before vaccination (neg. 1) or a non-specific antibody (neg. 2) was used (data not shown).

Neutralizing antibody response were measured two weeks after the last vaccination according to Kang et al., (FIG. 16 D).

As demonstrated by ELISA and SNT immunization with recombinant haploid yeast cells of the strain co-expressing four PRVA antigens induced protective neutralizing antibodies against PRVA.

### Example 11: Immunization of Mice with K. Marxianus Strain Expressing PPV-VP2 Antigen

The K. marxianus strains used for the immunization in this Example were produced according to the general procedure described in Examples 5 and 6.

TABLE 9
Haploid and diploid recombinant yeast cells used in this example

| Strain | MAT | Integrations | PPV antigens |
|---|---|---|---|
| SY61-haploid | haploid; | LAC4-LAC12: 2xVP2 | 2x VP2 |
| | MATa | ura3- | |
| SY61-diploid | diploid; | LAC4-LAC12: 2xVP2 | 2x VP2 |
| | MATα/MATa | | |
| SY135 | haploid; | LAC4-LAC12: 2xVP2 | 2x VP2 |
| | MATa | gal80Δ::ScURA3, | |
| | | ura3- | |
| SY41 | haploid; | ura3- | — |
| | MATa | | |

Groups of eight weeks old female BALB/c mice (n=5) were immunized subcutaneously with a single injection of a SY61-haploid or diploid strain (each 2 mg yeast dry weight), respectively (FIG. 17 A). Control animals received a wildtype strain SY41 without antigen (n=3). Sera at 2 weeks post immunization were collected and analyzed for PPV-VP2-specific IgG antibodies by an indirect ELISA (FIG. 17 B).

Mice immunized with haploid or diploid cells of K. marxianus each expressing two copies of codon-optimized VP2 variant of the NADL-2 strain (NP_757372.1) triggered a comparable immune response.

Groups of eight weeks old female BALB/c mice (n=5) were immunized subcutaneously three times with SY135 (expressing two copies of PPV-VP2) or the commercial vaccines Porciles® Parvo (MSD, Tiergesundheit), respectively, at day 0, 14 and 28 with 2 mg yeast dry weight (FIG. 17 C). Control animals received a wildtype strain SY41 without antigen (n=3). Sera were collected 14 days after each vaccination for measuring seroconversion. PPV-VP2-specific IgG antibody titres were determined by an indirect ELISA (FIG. 17 D). Data are one representative result of at least two independent experiments. Comparisons were performed by Student's t-test (unpaired, two tailed).

At day 14, day 28 and day 42, the mean titres of PPV-VP2-specific IgG in mice immunized with the K. marxianus strain SY135 expressing two copies of the PPV-VP2 gene were significantly higher than those observed in mice immunized with the commercial vaccine Porciles® Parvo (MSD, Tiergesundheit).

### Example 12: Immunization of Mice with K. marxianus Strain Expressing AHSV VP2 Antigen

The K. marxianus strain used for the immunization in this Example was produced according to the general procedure described in Examples 5 and 6.

TABLE 10
Recombinant yeast cells used in this example

| Strain | MAT | Integrations | AHSV antigens |
|---|---|---|---|
| SY338 | haploid; MATa | ADE2: 2xVP2 | 2x VP2 |
| | | gal80Δ::ScURA3, | (serotype 4) |
| | | met5Δ, ura3- | |
| SY41 | haploid; MATa | ura3- | — |

In this example, recombinant VP2 protein of serotype 4 (codon-optimized for K. marxianus) were expressed individually by the K. marxianus expression system (as described for PRVA) and the immunogenicity was studied by immunization of mice with single VP2. Homologous neutralizing antibodies measured by 50% plaque reduction assay showed degrees from 10 to 20 of titers for the VP2 protein.

The results indicate that AHSV VP2 subunit vaccines are efficient at raising an immune response individually or in a multi-serotype cocktail.

Eight weeks old female BALB/c mice were vaccinated subcutaneously three times with yeast strain SY338 (expressing two copies of AHSV-serotype 4 VP2) at day 0, 14 and 28 with 2 mg yeast dry weight (FIG. 18A). Control animals received a wildtype strain SY41 without antigen.

Mice sera collected at the end of the experiment, day 42, were examined for neutralizing antibodies by plaque reduction in presence of 100 TCID (Tissue Culture Infection Dose) of virus based on standard neutralizing assay (FIG. 18 B). The neutralization titers were calculated by the reciprocal value of the maximum dilution, at which the number of plaques showed 50% reduction compared with the serum-free control. The neutralizing tests were performed in duplicate.

1-15. (canceled)

16. A recombinant yeast cell comprising at least one genomically integrated expression cassette, wherein each expression cassette comprises
(i) a bidirectional promoter element;
(ii) a first transgene and a second transgene, wherein said first and second transgene are located at opposite ends of the bidirectional promoter element and wherein each transgene is operably linked to one side of the bidirectional promoter element;
(iii) a first transcription terminator and a second transcription terminator, said first transcription terminator being located immediately downstream of the first transgene and said second transcription terminator being located immediately downstream of the second transgene; wherein the first transcription terminator is operably linked to the first transgene and the second transcription terminator is operably linked to the second transgene; and
(iv) at least one selection marker,
wherein each transgene encodes an immunogenic polypeptide derived from a pathogen, or an immunogenic fragment thereof, and wherein the recombinant yeast cell is haploid and heterothallic.

17. The recombinant yeast cell according to claim 16, wherein the pathogen is a viral pathogen.

18. The recombinant yeast cell according to claim 17, wherein the pathogen is a viral pathogen belonging to the family of Reoviridae.

19. The recombinant yeast cell according to claim 18, wherein said viral pathogen is
a) a rotavirus, or
b) an orbivirus.

20. The recombinant yeast cell according to claim 19, wherein said viral pathogen is
a) Porcine Rotavirus A (PRVA); or
b) African Horse Sickness Virus (AHSV).

21. The recombinant yeast cell according to claim 17, wherein the viral pathogen is
a) Porcine Parvovirus (PPV); or
b) African Swine Fever Virus (ASFV).

22. The recombinant yeast cell according to claim 21, wherein the viral pathogen is
a) Porcine Parvovirus 1 (PPV1); or
b) African Swine Fever Virus (ASFV).

23. The recombinant yeast cell according to claim 16, wherein the bidirectional promoter element is
a) a native bidirectional yeast promoter element selected from the group consisting of a LAC4-LAC12 promoter, a GAL1-GAL10 promoter and a GAP1-ADH2 promoter; or
b) a hybrid bidirectional promoter element selected from the group consisting of a TDH3/ADHI promoter, a GAL1/GAL10-GPD promoter and a TEF1/PGK1 promoter.

24. The recombinant yeast cell according to claim 16, wherein the bidirectional promoter element enables inducible or repressible expression of the first and/or the second transgene.

25. The recombinant yeast cell according to claim 16, wherein each expression cassette is genomically integrated at a chromosomal locus selected from the group consisting of ADE1, ADE2, ADE8, ECM31, HIS2, HIS3, HIS5, LEU1, LEU2, LYS2, LYS5, MET5, MET17, TRP1, TRP3, TRP4, TRP5, TYR1, URA3, URA5, FCY1, GAP1, LAC4+LAC12, XYL1 and XYL2.

26. The recombinant yeast cell according to claim 16, wherein the selection marker is a resistance marker or a metabolic selection marker.

27. The recombinant yeast cell according to claim 26, wherein the selection marker is a metabolic selection marker.

28. The recombinant yeast cell according to claim 16, wherein the yeast cell comprises deletions and/or mutations rendering a gene required for mating type switching from MATa to MATα or a gene required for mating type switching from MATα to MATa non-functional.

29. A diploid recombinant yeast cell obtained by mating a first and a second haploid recombinant yeast cell according to claim 16.

30. The diploid recombinant yeast cell according to claim 29, comprising at least two selection markers genomically integrated at chromosomal loci located on homologous chromosomes.

31. A vaccine composition comprising at least one recombinant yeast cell according to claim 16.

32. The recombinant yeast cell according to claim 16, for use in vaccinating a subject against an infectious disease.

33. The vaccine composition according to claim 31, for use in vaccinating a subject against an infectious disease.

34. A method for the production of a whole yeast vaccine comprising at least one diploid recombinant yeast cell, the method comprising
generation of the diploid recombinant yeast cell by mating of a first and second haploid yeast cell according to claim 16,
wherein the first yeast cell is of mating type MATa and comprises a first expression cassette and the second yeast cell is of mating type MATa and comprises a second expression cassette, wherein the first and second expression cassette are stably integrated into the genome of the first and second yeast cell at the same chromosomal locus, wherein the first and second yeast cell are haploid, heterothallic and mating competent; and
wherein each transgene encodes an immunogenic polypeptide derived from a pathogen.

35. A method for the production of a diploid recombinant yeast cell from a wild type yeast strain comprising
(a) generating a first yeast cell of mating type MATa and a second yeast cell of mating type MATα from the wild type yeast strain, such that the first and second yeast cell are each heterothallic, isogenic and mating competent;
(b) transforming each of the first and second yeast cell with at least one deletion cassette such that the at least one deletion cassette stably integrates into the host cell genome at the same chromosomal locus in the first and in the second yeast cell;
such that stable integration into the yeast cell genome results in the disruption of a gene required for the synthesis of an essential compound, preferably wherein the essential compound is a vitamin, an amino acid, a nucleoside, a nucleobase or one or more metabolic precursor thereof;
(c) transforming each of the first and second yeast cell obtained in step (b) with a nucleic acid molecule comprising at least one expression cassette such that the at least one expression cassette stably integrates into the yeast cell genome at the chromosomal locus comprising the deletion cassette of step (b), such that the ability of the first and second yeast cell to synthesize the essential compound of step (b) is restored; and
(d) generating the diploid yeast cell by mating or spheroplast fusion of the first and second haploid yeast cell obtained in step (c);
wherein the wild-type yeast strain comprises no genetic selection marker and wherein each deletion cassette comprises at least one marker gene, preferably an auxotrophic marker gene; and optionally loxP sites flanking the marker gene; and wherein each expression cassette comprises
(i) a bidirectional promoter element;
(ii) a first transgene and a second transgene, wherein said first and second transgene are located at opposite ends of the bidirectional promoter element and wherein each transgene is operably linked to one side of the bidirectional promoter element;
(iii) a first transcription terminator and a second transcription terminator, said first transcription terminator being located immediately downstream of the first transgene and said second transcription terminator being located immediately downstream of the second transgene; wherein the first transcription terminator is operably linked to the first transgene and the second transcription terminator is operably linked to the second transgene; and
(iv) at least one selection marker; and
(v) a sequence for restoring the gene required for the synthesis of an essential compound of step (b),
wherein each transgene encodes an immunogenic polypeptide derived from a pathogen, or an immunogenic fragment thereof.


SEQUENCE LISTING (50 sequences)

SEQ ID NO: 1 - protein, 890 aa, Unknown
  Reoviridae
MAYRKRGAKRENSSQQNERLQEKEVEKNIDASMENKANNKKQQLPDKVLSQKEEIITDAQDDVKITDEVKKSSKEESKQLLEILKTKEDHQKEIQYEILQKTIPTFEPKESILKKLEDIKPEQAKKQTKLFRIFEPRQLPIYRANGERELRNRWYWKLKRDTLPDGDYDVREYFLNLYDQILIEMPDYLLLKDMAVENKNSRDAGKVVDSETASICDAIFQDEETEGVIRRFIADMRQQVQADRNVVNYPSILHPIDHAFNEYFLNHQLVEPLNNEIIFNYIPERIRNDVNYILNMDMNLPSTARYIRPNLLQDRLNLHDNFESLWDTITTSNYILARSVVPDLKEKELVSTEAQIQKMSQDLQLEALTIQSETQFLAGINSQAANDCFKTLIAAMLSQRTMSLEFVTTNYMSLISGMWLLTVIPNDMFLRESLVACELAIINTIVYPAFGMQRMHYRNGDPQTPFQIAEQQIQNFQVANWLHFINNNRFRQVVIDGVLNQTLNDNIRNGQVINQLMEALMQLSRQQFPTMPVDYKRSIQRGILLLSNRLGQLVDLTRLLSYNYETLMACITMNMQHVQTLTTEKLQLTSVTSLCMLIGNTTVIPSPQTLFHYYNVNVNFHSNYNERINDAVAIITAANRLNLYQKKMKSIVEEFLKRLQIFDVPRVPDDQMYRLRDRLRLLPVERRRLDIFNLILMNMEQIERASDKIAQGVIIAYRDMQLERDEMYGYVNIARNLDGYQQINLEELMRTGDYGQITNMLLNNQPVALVGALPFVTDSSVISLIAKLDATVFAQIVKLRKVDTLKPILYKINSDSNDFYLVANYDWIPTSTTKVYKQVPQPFDFRASMHMLTSNLTFTVYSDLLAFVSADTVEPINAVAFDNMRIMNEL

SEQ ID NO: 2 - protein, 776 aa, Unknown
  Reoviridae
MASLIYRQLLTNSYTVNLSDEIQEIGSAKSQDVTINPGPFAQTGYAPVNWGAGETNDSTTVEPLLDGPYQPTTFNPPTSYWVLLAPTVEGVIIQGTNNTDRWLATILIEPNVQTTNRIYNLFGQQVTLSVENTSQTHWKFIDVSTTTPTGSYTQHGPLFSTPKLYAVMKFSGRIYTYNGTTPNATTGYYSTTNYDTVNMTSFCDFYIIPRNQEEKCTEYINHGLPPIQNTRNVVPVSLSAREIVHTRAQVNEDIVVSKTSLWKEMQCNRDITIRFKFDRTIIKAGGLGYKWSEISFKPITYQYTYARDGEQITAHTTCSVNGVNNFSYNGGSLPTDFAISRYEVIKENSFVYIDYWDDSQAFRNMVYVRSLAANLNTVTCTGGSYSFALPLGHYPVMTGGTVSLHPAGVTLSTQFTDFVSLNSLRFRFRLTVGEPSFSITRTRVSRLYGLPAANPNNQREYYEISGRFSLISLVPSNDDYQTPIMNSVTVRQDLERQLGELRDEFNSLSQQIAISQLIDLALLPLDMFSMFSGIKSTIDAAKSMATNVMKRFKRSNLASSVSTLTDAMSDAASSISRSSSIRSIGSSASAWTEVSNSIADVSTTVDTVSTQTATIAKRLRLKEIATQTDGMNFDDISAAVLKTKIDKSVQITPNTLPEIVTEASEKFIPNRTYRVINNDEVFEVGMDGKFFAYRVDTFDEIPFDVQKFADLVTDSPVISAIIDLKTLKNLKDNYGISKQQAFDLLRSDPRVLREFINQNNPIIRNRIENLIMQCRL

SEQ ID NO: 3 - protein, 397 aa, Unknown
  Reoviridae
MEVLYSLSKTLKDARDKIVEGTLYSNISDLIQQFNQMIVTMNGNDFQTGGIGNLPIRNWNFDFGLLGTTLLNIDANYVENARTTIEYFIDFIDNVCMDEMARESQRNGIAPQSEALRKLSGIKFKRINFDNSSDYIENWNLQNRRQRTGFVFHKPNILPYSASFTLNRSQPAHDNLMGTMWINAGSEIQVAGFDYSCAFNAPANIQQFEHVVPLRRALTTATITLLPDAERFSFPRVINSADGATTWYFNPVIIRPSNVEVEFLLNGQIINTYQARFGTIIARNFDTIRLSFQLVRPPNMTPAVANLFPQAPPFIFHATVGLTLRIESAVCESVLADASETLLANVTAVRQEYAIPVGPVFPPGMNWTELITNYSPSREDNLQRVFTVASIRSMLIK

SEQ ID NO: 4 - protein, 326 aa, Unknown
  Reoviridae
MYGIEYTTVLTFLISLVFVNYILKSVTRTMDFIIYRFLLVIVVLAPLIKAQNYGINLPITGSMDTPYMNSTTSETFLTSTLCLYYPNEAATEIADTKWTETLSQLFLTKGWPTGSVYFKGYADIASFSVEPQLYCDYNIVLMKYDGNLQLDMSELADLILNEWLCNPMDIMLYYYQQTDEANKWISMGTSCTIKVCPLNTQTLGIGCSTTDINSFETVANAEKLAITDVVDGVNHKLDVTTSTCTIRNCKKLGPRENVAVIQVGGPNILDITADPTTAPQTERMMRINWKRWWQVFYTIVDYVNQIVQVMSKRSRSLDSAAFYYRV

SEQ ID NO: 5 - protein, 317 aa, Unknown
  Reoviridae
MAELACFCYPHLENDSYKFIPFNNLAIKCMLTAKVDKKDQDKFYNSIVYGIAPPPQFKKRYNTSDNSRGMNYVTTMFNKVAILICEALNSIKVTQSDVANVLSRVVSVRHLENLVLRKENHQDVLFHSKELLLKSVLIAIGQSKEIETTATAEGGEIVFQNAAFTMWKLTYLDHKLMPILDQNFTEYKITLNEDKPISDVCVKELVAELRWQYNRFAVITHGKGHYRVVKYSSVANHADRVFATYKNNAKSGNVTDFNLLDQRIIWQNWYAFTSSMKQGNTLDVCKKLLFQKMKQEKNPFKGLSTDRKMDEVSHVGI

SEQ ID NO: 6 - protein, 175 aa, Unknown
  Reoviridae
MDKLADLNYTLSVITLMNDTLHSIIQDPGMAYFPYIASVLTVLFTLHKASIPTMKIALKTSKCSYKVIKYCMVTIINTLLKLAGYKEQVTTKDEIEQQMDRIIKEMRRQLEMIDKLTTREIEQVELLKRIHDKLAARSVDAIDMSKEFNQKNIRTLDEWESGKNPYEPSEVTASM

SEQ ID NO: 7 - DNA, 2676 bp, Unknown
  Reoviridae
tactaacagttataccaaacgacagctaaacgtgaaaactcatcacaacagaatgaacgtctgcaagaaaaagaagttgaaaagaacatagacgcatctatggagaataaagctaataataaaaagcaacaattacctgataaagtgttatcacaaaaagaagagataataactgacgcacaagatgatgttaagataactgatgaggttaagaaatcatcaaaagaagaatcgaaacagcttttggagatattgaaaactaaagaagatcatcagaaagagatacaatatgaaattttacagaaaacgataccaacttttgaaccaaaagaatcaattttgaaaaaattagaagacataaaaccagaacaggcaaaaaaacaaactaaattgtttagaatatttgaaccaagacaattaccaatttatcgggcaaatggtgagagagaattgagaaatagatggtattggaaattgaaaagagacacgctgccagatggagattatgatgtccgagaatacttcttaaatttatatgatcagatattgatagaaatgccagattatttattattgaaagatatggcagtggagaataaaaattctagggatgctggtaaagtcgtggattccgagacagcaagtatttgtgatgccatatttcaagatgaggaaacagagggagtcattagaaggttcattgcagatatgaggcaacaagttcaagctgacagaaatgttgttaactatccatcaattttacatccaattgatcatgcattcaatgaatactttttaaatcaccaattagttgaaccattaaataatgaaataatttttaattatataccagaaaggataagaaacgatgttaattacattctaaacatggatatgaatctaccatcaacagctagatatattagaccaaatttgctgcaagatagactaaatttgcatgataattttgaatcattatgggatacgataacaacatcaaactatatattagctagatcagttgtgcctgatctgaaggaaaaggagttagtgtcaaccgaagctcaaatacagaaaatgtcccaagatttgcaacttgaagcactaactatacaatctgaaacgcagtttctggctggtataaattcacaagcagcaaatgattgttttaaaacgctaatagcagccatgttaagccaacgtacaatgtcattagaatttgtgactacaaattacatgtcacttatatctggtatgtggttactaacagttataccaaacgacatgtttcttcgtgaatcgctagttgcatgcgagttggctataataaacactattgtttatccagcgttcggaatgcaaaggatgcattatagaaatggtgatccacagactccattccaaatagcagaacagcagatacaaaattttcaagtagctaattggctacactttatcaacaataatagatttagacaagttgtgatcgacggagtgttgaatcaaacacttaatgataatattagaaatggacaagttattaatcagttgatggaagcgttaatgcaactatctagacaacagtttccaactatgccagttgattataaaagatcaatacaaagaggaatattactattgtcaaacagattaggtcaattagttgacttaacaagactattatcatataactatgaaacactgatggcttgcataaccatgaatatgcaacatgtacaaactcttactactgaaaaattacaattaacttctgttacatctttgtgtatgttaattggaaatactacagttattccaagtccacaaacattgtttcactattacaatgtaaatgtaaattttcattcaaactataacgaacggattaacgatgcagtagcaattattacagcagccaatagactgaatttatatcagaagaagatgaagtcaatagttgaagaatttttgaagaggctgcaaattttcgatgtgccacgagtgcccgatgatcaaatgtatagactgagagatagacttagattgttgccggttgaaagacggagacttgatatattcaatttaatattaatgaatatggagcagatcgagcgagcttcagataaaattgcacaaggtgtaataattgcttatagagatatgcaactagagagagatgaaatgtatggatacgtcaatattgctagaaatctggatggataccaacaaattaatttagaagaattaatgaggacaggagattatggtcaaattactaacatgctattgaacaaccagccagtggctctagtaggagcattgccattcgtaacagactcatcagttatatcactcattgctaagctggatgctacagttttcgcccagatagtcaaacttaggaaagtagataccttaaaaccgatattgtataaaataaactctgattctaatgatttctatttagttgcaaattacgattggataccaacttccactacaaaagtgtacaaacaggtaccgcaaccctttgactttagagcgtcaatgcatatgttaacatctaacctcacttttactgtttattccgatttgttagcttttgtttctgcagatacagttgaaccgatcaacgcagttgcttttgacaatatgcgcattatgaacgaactgtaa

SEQ ID NO: 8 - DNA, 2331 bp, Unknown
  Reoviridae
atggcttcgctcatttatagacaactacttactaattcatacacagtcaatctttctgacgaaattcaagagattggatcggctaagtcacaggatgttactataaatcctggtccattcgcacaaacaggttatgcaccagttaattggggagcaggtgagactaatgactccacaactgtcgagccgttattagatggtccataccaaccaaccactttcaatccaccaacaagctattgggtactacttgcgccaactgtagagggcgtaattattcaaggaacaaacaataccgatagatggttggccactatactaattgaaccaaacgtacaaacaactaacagaatatacaatctttttggtcagcaagtaactttatcggtggagaatacgtcacagacacattggaagttcattgatgtgagtacaactacgccaacaggaagttatacgcagcacggaccattgttctctacaccaaaattatacgctgtaatgaaattcagtggtagaatatatacatataatggaaccacaccaaacgcaacaacaggatactattcaactactaattatgacacagtaaatatgacatcattttgtgatttttatattataccaagaaatcaagaagaaaaatgtactgagtatatcaatcatggattacctcctatacaaaatacaaggaatgttgtgccagtatctttatcggctagagagatagtgcacacaagagctcaagttaatgaggatattgttgtttcaaaaacttcactttggaaagaaatgcaatgcaacagagacataaccataagattcaaatttgatagaacaattattaaagctggaggattaggatacaaatggtcagaaatatcttttaagccaattacttatcagtacacatacgctagagatggagaacaaattacagcgcacaccacatgctcagttaatggagttaacaattttagttacaatggcggttcgctgccgacggacttcgccatatcaagatatgaagtgattaaagaaaattcatttgtttacattgattactgggatgattcacaagcattcagaaacatggtatatgttcggtcacttgctgctaatttgaatacagtaacgtgtactggtggtagttacagcttcgcgttgcctttaggtcattatccagttatgactggtggtacagtttcactacacccagctggagttacattatctacccaattcactgatttcgtatctcttaattcattacgctttagattcaggttaactgtaggagaaccttcattttctataacgaggactagggtaagtaggttatacggacttccagcagctaatccaaacaaccaaagagaatattatgaaataagcggtaggttttcattaatatcattagtgccatcaaatgatgattatcaaacaccaattatgaattcagttactgtgaggcaagatctagagagacaattgggagagctacgagatgagttcaattcattgtcacagcaaatagcgatatcacaattgattgatctggctctattgccattagatatgttttcaatgttctcaggaattaaaagtacaatagatgctgcgaaatccatggcaacgaacgtgatgaaaagatttaaacggtcaaacctagccagctcagtttctacattaactgatgcaatgtctgatgcagcatcgtctatttcaagaagttcatcaatacgatcaataggatcatcagcatccgcttggacggaagtgtcaaattcgatcgcagatgtttctacgacagttgatacagtatcaacgcaaactgctactattgctaagcggttacgattgaaagagattgcaactcaaaccgatggtatgaattttgatgacatatctgcagcagtgttaaaaacgaaaattgacaaatccgtgcaaataacaccaaatacattaccggaaatagttactgaagcttctgagaaatttatcccaaatagaacatatagggttattaacaatgatgaagtatttgaagttggaatggatggtaagtttttcgcatatcgtgtagacacattcgatgaaataccatttgatgtacagaaattcgcagatttagttacagattcaccagtcatttctgcaataattgatcttaagacgttaaaaaatctgaaagataattatggaataagtaagcagcaagcttttgacttactacgatcagatccaagggtattacgtgaatttatcaatcagaataatccaataatacgaaatagaattgaaaacttaatcatgcaatgtagactgtaa

SEQ ID NO: 9 - DNA, 1194 bp, Unknown
  Reoviridae
atggaggttctgtactcattgtcaaaaaccttgaaagatgctagagataaaattgttgaaggtacattatactcaaatataagtgatttaattcaacaattcaatcaaatgatagttaccatgaatggaaatgattttcaaacgggaggaataggaaatttgccaatcagaaactggaattttgatttcggattacttggcactactttacttaatatagatgcaaattatgttgaaaatgctagaactaccattgaatatttcattgattttatagataatgtgtgtatggatgaaatggctagagagtcgcaacgaaacggaatagctccacaatctgaagcactgagaaagctgtcaggtattaagtttaagagaattaattttgacaattcatctgattacattgagaattggaatttacaaaatagacgacagcgtactggattcgtgttccataagccaaatatacttccatactcagcatcattcactttgaatagatcacagccggcacatgataatttaatggggactatgtggattaacgctggatcagaaattcaggtggctggatttgattattcgtgtgcttttaatgcaccggcaaatattcagcagtttgaacatgtcgtgccattaagacgtgcacttacgacagctacaattactttgctaccagatgctgagaggttcagtttcccaagagtgattaattcagctgatggcgctactacatggtacttcaatccagttatcataagaccaagtaatgttgaagttgaatttttgttgaatgggcaaataattaatacgtaccaagcgcgatttggaaccatcatagctagaaattttgatactattcggttatcatttcaattagtacgaccaccgaatatgacaccagcagttgcaaacctatttccgcaagcaccaccatttatatttcatgctacagttggactcacactacgaattgaatctgcagtttgtgaatctgtgcttgcggacgcttccgaaactttattggcaaatgtgactgcagttcgtcaagagtatgctataccggtagggccagtgttcccaccaggtatgaattggacagaattgattaccaattattcaccttcaagagaagataacttgcaacgtgttttcacagtagcttccattagaagcatgttgattaagtga

SEQ ID NO: 10 - DNA, 981 bp, Unknown
  Reoviridae
atgtatggtattgaatataccacagttctaacttttttgatatcgcttgtatttgtcaattatatactgaaatcagttactagaacaatggactttatcatttatagattcttattggttatagtcgtacttgcaccgctcattaaagctcaaaattacggaattaatttaccaataactggatctatggatacgccatatatgaattcaactacaagtgaaacatttttgacttcgacattatgtctatattatccaaatgaagcagctacagaaattgcagatacaaaatggacagaaacattgtcgcagttgtttttaacaaaaggatggccaacagggtcagtttattttaaaggatatgcagatattgcgtcattttctgtagaaccgcagttatactgcgactataatattgtactaatgaaatatgatggaaatttacagttagacatgtctgaattggctgatttaatattgaatgaatggctatgtaatccaatggatataatgctatattattatcagcaaacagatgaagctaataaatggatatcaatgggtacatcatgtacgattaaagtatgtcctctaaatacgcagactctcgggataggatgttcgactacagacataaattcatttgaaacagtggccaatgcagagaaattagctataactgatgttgtcgatggagtcaatcataaattagacgtaacaacgagtacatgtactataagaaattgtaaaaaacttggaccaagagaaaatgtcgctgtaattcaggtaggaggtccaaacatactcgacataacagctgatccaacaactgcaccacaaactgaaagaatgatgcgtataaattggaagagatggtggcaagtcttttatacaatagttgattatgtcaatcaaattgtacaagtcatgtccaagcgatcacgctccttagattctgctgccttttattaccgagtctag

SEQ ID NO: 11 - DNA, 954 bp, Unknown
  Reoviridae
atggctgagctagcttgcttttgttatcctcatttggagaacgatagctataaatttattccttttaataatttagcaataaaatgcatgttgacagctaaagtggataaaaaagatcaagataaattttacaattctattgtttatggaattgcgccaccaccacaatttaaaaaacgttataatactagtgataattcaaggggcatgaattatgtaacgactatgtttaataaggtggccatcttaatttgtgaagcgctcaattcaattaaagttacccaatctgacgttgctaatgttctttcaagagtggtttccgttagacatttggaaaatttggtgttaagaaaagaaaaccatcaagatgtgctgtttcattcgaaagaactactcttaaaatctgttttgatagctattggtcaatcaaaagaaattgaaactactgccactgctgaaggaggagaaatagtatttcagaatgcagcttttactatgtggaaattgacgtatttagatcataaattgatgcctattttggatcagaacttcactgaatataaaattacattgaatgaagataaaccaatttcagatgtatgtgttaaagaacttgtcgctgaattgagatggcagtacaacagatttgctgtaataacacatggtaaaggtcactatagagttgttaaatattcatcagttgctaatcatgcagacagagtgtttgctacatataaaaataatgctaagagtggcaatgttactgactttaacttgttggaccaaagaattatttggcaaaattggtatgcttttacatcctcaatgaaacaaggtaatacacttgatgtatgtaagaaactgctcttccaaaagatgaaacaggagaaaaatccgttcaaaggactgtcaactgatagaaaaatggatgaagtttcacatgttggaatttaa

SEQ ID NO: 12 - DNA, 528 bp, Unknown
  Reoviridae
atggataagcttgccgacctcaattacacattgagcgtaatcactttaatgaatgacacactacactctattattcaagatccaggaatggcgtattttccatatattgcatctgttctgactgttttatttactctacataaagcatcaattccaacaatgaaaatagcgttaaaaacgtcaaagtgttcgtacaaagtaattaaatattgcatggttacaatcattaatactcttctgaagttggctggttataaagaacaggttactactaaggatgaaattgaacaacagatggacagaattattaaagagatgagacgtcaactggaaatgattgacaaattgacaactcgtgaaattgaacaggttgaattacttaaacgtatacatgacaaattagctgctagatcagttgacgctatagatatgtcgaaagaatttaatcagaaaaatattcgaacgctagatgaatgggaaagtggaaaaaatccatatgaaccgtcggaagtaactgcgtctatgtga

SEQ ID NO: 13 - DNA, 2673 bp, Artificial Sequence
  PRVA VP2 optimized
atggcatacagaaagagaggtgcaaaaagagaaaactcatcacaacaaaacgaaagattacaagaaaaggaagtagaaaagaacattgatgcatctatggaaaataaggctaacaataagaaacaacaattgccagataaggttttgtcacaaaaggaagaaatcatcactgatgctcaagatgatgttaagatcacagatgaagttaagaaatcttcaaaagaagaatctaagcaattgttggaaattttgaagactaaagaagatcatcaaaaagaaattcaatacgaaattttacaaaagactatcccaacattcgaaccaaaggaatcaatcttgaagaaattggaagatatcaagccagaacaagctaagaaacaaactaagttgtttagaatcttcgaaccaagacaattgccaatctatagagctaatggtgaaagagaattgagaaacagatggtactggaagttgaagagagatacattgccagatggtgactatgatgttagagaatactttttgaatttgtacgatcaaattttaatcgaaatgccagattatttgttgttgaaggatatggcagttgaaaataagaactctagagatgctggtaaagttgttgattctgaaactgcatcaatctgtgatgctatcttccaagatgaagaaacagaaggtgttattagaagattcattgcagatatgagacaacaagttcaagctgatagaaacgttgttaactacccatctatcttgcatccaatcgatcatgcttttaatgaatactttttgaaccatcaattggttgaaccattgaacaacgaaatcattttcaattacattccagaaagaattagaaatgatgttaattatattttgaatatggatatgaatttgccatctactgctagatacattagaccaaatttgttgcaagatagattgaatttgcatgataacttcgaatcattgtgggatactatcactacatctaactacatcttggcaagatcagttgttccagatttgaaggaaaaggaattagtttctacagaagctcaaatccaaaagatgtcacaagatttgcaattggaagcattgactatccaatctgaaacacaattcttggctggtattaattcacaagctgcaaacgattgtttcaagactttgatcgctgcaatgttgtctcaaagaacaatgtcattggaattcgttactacaaactacatgtctttgatttcaggcatgtggttgttgactgttattccaaacgatatgttcttgagagaatctttggttgcttgtgaattggcaattattaacactatcgtttacccagctttcggtatgcaaagaatgcattacagaaatggtgacccacaaacaccatttcaaattgcagaacaacaaatccaaaacttccaagttgctaactggttgcatttcatcaacaacaacagattcagacaagttgttattgatggtgttttgaaccaaactttgaacgataacatcagaaatggtcaagttattaatcaattgatggaagctttgatgcaattgtctagacaacaattcccaacaatgccagttgattacaagagatcaatccaaagaggtatcttattgttgtcaaacagattgggtcaattggttgatttgactagattgttgtcatacaactacgaaactttgatggcttgtatcacaatgaacatgcaacatgttcaaacattgactacagaaaagttgcaattgacttctgttacatcattgtgtatgttgatcggtaacactacagttattccatctccacaaactttgttccattactacaacgttaacgttaacttccattcaaattacaatgaaagaattaatgatgctgttgcaatcatcacagctgcaaacagattgaatttgtaccaaaagaaaatgaaatctattgttgaagaatttttaaaaagattgcaaatttttgatgttccaagagttccagatgatcaaatgtacagattgagagatagattgagattgttgccagttgaaagaagaagattggatatttttaatttgatcttgatgaatatggaacaaatcgaaagagcatctgataagatcgctcaaggtgttattatcgcatacagagatatgcaattggaaagagatgaaatgtacggttacgttaacatcgctagaaatttggatggttaccaacaaattaatttggaagaattgatgagaactggtgactatggtcaaatcacaaacatgttgttgaacaaccaaccagttgctttggttggtgcattgccattcgttactgattcttcagttatttctttgatcgctaagttggatgcaactgttttcgctcaaatcgttaagttgagaaaagttgatacattgaaaccaatcttgtacaagattaattctgattcaaatgatttttacttagttgcaaattatgattggatcccaacatcaactacaaaggtttacaagcaagttccacaaccattcgatttcagagcttctatgcatatgttgacttcaaatttgacttttacagtttactctgatttgttggcatttgtttcagctgatacagttgaaccaattaatgctgttgcattcgataacatgagaatcatgaacgaattgtga

SEQ ID NO: 14 - DNA, 2331 bp, Artificial Sequence
  PRVA VP4 optimized
atggcttctttgatctatagacaattgttgactaactcatacacagttaatttgtctgatgaaatccaagaaatcggttctgctaagtctcaagatgttactattaatccaggtccatttgctcaaacaggttacgcaccagttaattggggtgcaggtgaaactaatgattctactacagttgaaccattgttagatggtccataccaaccaactacttttaatccaccaacttcttattgggttttgttagctccaacagttgaaggtgttattatccaaggtacaaacaacactgatagatggttggcaactatcttgatcgaaccaaacgttcaaactacaaacagaatctataatttgtttggtcaacaagttacattgtcagttgaaaacacatctcaaactcattggaagtttattgatgtttcaactacaactccaactggttcttacacacaacatggtccattattttcaactccaaagttgtacgctgttatgaaattttctggtagaatctatacatacaatggtacaactccaaatgcaacaactggttactactcaacaactaactacgatactgttaacatgacatctttctgtgatttctacattattccaagaaaccaagaagaaaagtgtactgaatacatcaaccatggtttaccaccaatccaaaacacaagaaacgttgttccagtttcattgtctgctagagaaattgttcatactagagcacaagttaacgaagatatcgttgtttcaaagacatctttatggaaagaaatgcaatgtaacagagatatcactatcagattcaaattcgatagaacaattattaaggctggtggtttgggttataaatggtcagaaatctcttttaaaccaatcacttaccaatacacatacgctagagatggtgaacaaattactgcacatacaacttgttctgttaacggtgttaacaacttctcatacaacggtggttctttgccaacagatttcgctatctcaagatacgaagttattaaagaaaattcttttgtttacattgattattgggatgattcacaagcttttagaaacatggtttacgtcagatctttggctgcaaatttgaatacagttacttgtacaggtggttcatactcttttgctttgccattaggtcattatccagttatgacaggtggtactgtttcattacatccagcaggtgttacattgtctactcaattcactgatttcgtttcattgaactctttgagattcagattcagattaactgttggtgaaccatcattttctatcactagaacaagagtttctagattgtacggtttaccagctgcaaatccaaacaaccaaagagaatactacgaaatctcaggtagattttcattgatctctttggttccatctaacgatgattaccaaactccaatcatgaactcagttacagttagacaagatttggaaagacaattgggtgaattgagagatgaattcaattcattgtctcaacaaatcgctatctctcaattgatcgatttggcattgttgccattggatatgttctcaatgttctctggtattaaatcaactatcgatgctgcaaagtctatggctacaaacgttatgaagagattcaaaagatcaaatttggcttcttcagtttctactttgacagatgcaatgtcagatgctgcatcttcaatctctagatcttcatctatcagatcaattggttcatctgcttctgcatggactgaagtttcaaattctatcgctgatgtttcaacaactgttgatacagtttctactcaaacagctactatcgcaaagagattgagattgaaggaaatcgcaacacaaactgatggtatgaacttcgatgatatctctgctgcagttttgaagactaagatcgataagtcagttcaaattactccaaatacattgccagaaatcgttactgaagcttctgaaaagtttattccaaacagaacatacagagttattaataacgatgaagttttcgaagttggtatggatggtaaatttttcgcttacagagttgatactttcgatgaaatcccattcgatgttcaaaagttcgctgatttggttactgattcaccagttatttctgcaatcatcgatttgaagacattgaaaaatttgaaagataattatggtatctcaaagcaacaagctttcgatttgttaagatctgatccaagagttttgagagaattcattaaccaaaacaacccaatcatcagaaatagaattgaaaatttgattatgcaatgtagattgtaa

SEQ ID NO: 15 - DNA, 1194 bp, Artificial Sequence
  PRVA VP6 optimized
atggaagttttgtactctttgtccaagactttgaaggatgccagagataagatcgttgaaggtactttgtactctaacatttctgatttgatccaacaattcaaccaaatgattgttaccatgaacggtaacgacttccaaactggtggtatcggtaacttgccaattagaaactggaacttcgacttcggtttgttgggtaccactttgttgaacatcgatgctaactacgttgaaaacgctagaaccactatcgaatacttcatcgacttcatcgataacgtttgtatggatgaaatggctagagaatctcaaagaaacggtatcgctccacaatctgaagccttgagaaagttgtccggtatcaagttcaagagaattaacttcgacaactcttccgattacatcgaaaactggaacttgcaaaacagaagacaaagaaccggtttcgttttccacaagccaaacattttgccatactctgcttccttcaccttgaacagatcccaaccagctcacgacaacttgatgggtactatgtggatcaacgctggttctgaaattcaagttgctggtttcgattactcctgtgctttcaacgctccagctaacatccaacaattcgaacacgttgttccattgagaagagctttgaccactgctaccatcactttgttgccagacgctgaaagattctctttcccaagagttattaactccgctgatggtgctaccacttggtacttcaacccagttatcattagaccatctaacgttgaagttgaatttttgttgaacggtcaaatcattaacacctaccaagctagattcggtactatcattgctagaaacttcgacaccatcagattgtccttccaattggttagaccaccaaacatgactccagctgttgctaacttgttcccacaagctccaccattcatcttccacgctaccgttggtttgactttgagaattgaatctgctgtttgtgaatccgttttggctgatgcttctgaaaccttgttggctaacgttactgctgttagacaagaatacgctatcccagttggtccagttttcccaccaggtatgaactggaccgaattgattactaactactctccatccagagaagacaacttgcaaagagttttcaccgttgcttctatcagatccatgttgatcaagtaa

SEQ ID NO: 16 - DNA, 981 bp, Artificial Sequence
  PRVA VP7 optimized
atgtacggtatcgaatacactacagttttgacatttttgatttctttggttttcgttaactacatcttgaagtcagttactagaacaatggattttattatctatagatttttgttagttattgttgttttggctccattgattaaagcacaaaactacggtattaatttgccaatcactggttctatggatacaccatacatgaactctactacatcagaaacatttttgacttcaacattgtgtttgtactacccaaacgaagctgcaactgaaattgctgatacaaagtggactgaaacattgtctcaattgtttttaactaaaggttggccaacaggttcagtttacttcaagggttacgctgatatcgcatctttttcagttgaaccacaattgtactgtgattacaacatcgttttgatgaaatatgatggtaatttgcaattggatatgtctgaattggctgatttgatcttgaacgaatggttgtgtaacccaatggatatcatgttgtactactaccaacaaactgatgaagcaaataagtggatctctatgggtacttcatgtacaattaaagtttgtccattgaatactcaaacattgggtattggttgttctactacagatatcaactcattcgaaactgttgctaacgcagaaaagttggctatcacagatgttgttgatggtgttaaccataagttggatgttactacatcaacttgtacaatcagaaactgtaagaaattgggtccaagagaaaatgttgcagttattcaagttggtggtccaaatattttggatattactgctgatccaactacagcaccacaaacagaaagaatgatgagaattaattggaagagatggtggcaagttttctatactatcgttgattacgttaaccaaatcgttcaagttatgtctaagagatctagatcattggattcagctgcattttactatagagtttaa

SEQ ID NO: 17 - DNA, 954 bp, Artificial Sequence
  PRVA NSP2 optimized
atggccgaattagcatgtttctgttacccccacttagagaatgatagttacaagtttatacccttcaacaatctggcaattaagtgtatgcttacagccaaagtggacaagaaggatcaagataagttctataatagcatagtctatggtatagcccctcctcctcagttcaaaaagaggtataatacttcggataactcaagaggcatgaactatgtaaccaccatgttcaacaaggtggccattttgatttgcgaggctctaaactcaattaaagtgacacagagcgatgttgcaaatgtgttatcaagggtagtctcagtgaggcaccttgaaaacctggtgttgaggaaagagaaccatcaggatgtactgttccactcaaaagaactactacttaagtctgttttgatagcaataggacaatcgaaagaaatagaaacaactgctacggccgagggcggtgagatcgtgtttcagaatgctgctttcacaatgtggaagctaacatacctagaccataaactgatgcctattttagatcagaattttaccgagtataagattactcttaacgaggataagccaatcagtgatgtatgtgtaaaggagttggttgctgagcttcgttggcaatataacagattcgccgttataacccacggcaaaggacattacagagtcgtgaagtatagctcagtcgctaaccatgccgacagggtatttgccacctataagaataacgcaaaatctggcaatgttacagatttcaacttgctagaccagaggataatatggcaaaattggtacgcttttacgtcctccatgaaacagggaaatacgctagatgtctgcaagaaattgcttttccagaaaatgaagcaggagaagaacccctttaagggactatcgaccgatagaaaaatggacgaggtctcccacgtaggcatttga

SEQ ID NO: 18 - DNA, 528 bp, Artificial Sequence
  PRVA NSP4 optimized
atggataaacttgctgacctgaattataccttgtcggttataacgttaatgaatgacaccttgcattctattatccaagaccccggtatggcctacttcccttacattgcatcagtgttaaccgtgctattcacgttgcacaaagcatccatacccacgatgaaaatagcattaaagacctccaagtgttcgtataaagtcattaagtactgtatggtcactatcatcaatactcttctaaaattggctggatacaaagagcaggtcaccacgaaagatgaaatagaacagcagatggataggattatcaaagaaatgcgtaggcagttagaaatgatcgacaagttaacaacgcgtgaaatcgaacaggtagaattgctaaagagaatacatgacaagttagccgctaggtccgttgacgcaatagatatgtcgaaggagttcaatcaaaagaacatcaggactctggacgagtgggaaagcggaaagaacccctacgagccaagtgaggtaactgcctcgatgtag

SEQ ID NO: 19 - protein, 934 aa, Artificial Sequence
  PRVA VP2 modified
MAYRKRGAKRENSSQQNERLQEKEVEKNIDASMENKANNKKQQLPDKVLSQKEEIITDAQDDVKITDEVKKSSKEESKQLLEILKTKEDHQKEIQYEILQKTIPTFEPKESILKKLEDIKPEQAKKQTKLFRIFEPRQLPIYRANGERELRNRWYWKLKRDTLPDGDYDVREYFLNLYDQILIEMPDYLLLKDMAVENKNSRDAGKVVDSETASICDAIFQDEETEGVIRRFIADMRQQVQADRNVVNYPSILHPIDHAFNEYFLNHQLVEPLNNEIIFNYIPERIRNDVNYILNMDMNLPSTARYIRPNLLQDRLNLHDNFESLWDTITTSNYILARSVVPDLKEKELVSTEAQIQKMSQDLQLEALTIQSETQFLAGINSQAANDCFKTLIAAMLSQRTMSLEFVTTNYMSLISGMWLLTVIPNDMFLRESLVACELAIINTIVYPAFGMQRMHYRNGDPQTPFQIAEQQIQNFQVANWLHFINNNRFRQVVIDGVLNQTLNDNIRNGQVINQLMEALMQLSRQQFPTMPVDYKRSIQRGILLLSNRLGQLVDLTRLLSYNYETLMACITMNMQHVQTLTTEKLQLTSVTSLCMLIGNTTVIPSPQTLFHYYNVNVNFHSNYNERINDAVAIITAANRLNLYQKKMKSIVEEFLKRLQIFDVPRVPDDQMYRLRDRLRLLPVERRRLDIFNLILMNMEQIERASDKIAQGVIIAYRDMQLERDEMYGYVNIARNLDGYQQINLEELMRTGDYGQITNMLLNNQPVALVGALPFVTDSSVISLIAKLDATVFAQIVKLRKVDTLKPILYKINSDSNDFYLVANYDWIPTSTTKVYKQVPQPFDFRASMHMLTSNLTFTVYSDLLAFVSADTVEPINAVAFDNMRIMNELGGGGSGGGGSGYPYDVPDYAGYPYDVPDYAGSYPYDVPDYAAQK

SEQ ID NO: 20 - protein, 820 aa, Artificial Sequence
  PRVA VP4 modified
MASLIYRQLLTNSYTVNLSDEIQEIGSAKSQDVTINPGPFAQTGYAPVNWGAGETNDSTTVEPLLDGPYQPTTFNPPTSYWVLLAPTVEGVIIQGTNNTDRWLATILIEPNVQTTNRIYNLFGQQVTLSVENTSQTHWKFIDVSTTTPTGSYTQHGPLFSTPKLYAVMKFSGRIYTYNGTTPNATTGYYSTTNYDTVNMTSFCDFYIIPRNQEEKCTEYINHGLPPIQNTRNVVPVSLSAREIVHTRAQVNEDIVVSKTSLWKEMQCNRDITIRFKFDRTIIKAGGLGYKWSEISFKPITYQYTYARDGEQITAHTTCSVNGVNNFSYNGGSLPTDFAISRYEVIKENSFVYIDYWDDSQAFRNMVYVRSLAANLNTVTCTGGSYSFALPLGHYPVMTGGTVSLHPAGVTLSTQFTDFVSLNSLRFRFRLTVGEPSFSITRTRVSRLYGLPAANPNNQREYYEISGRFSLISLVPSNDDYQTPIMNSVTVRQDLERQLGELRDEFNSLSQQIAISQLIDLALLPLDMFSMFSGIKSTIDAAKSMATNVMKRFKRSNLASSVSTLTDAMSDAASSISRSSSIRSIGSSASAWTEVSNSIADVSTTVDTVSTQTATIAKRLRLKEIATQTDGMNFDDISAAVLKTKIDKSVQITPNTLPEIVTEASEKFIPNRTYRVINNDEVFEVGMDGKFFAYRVDTFDEIPFDVQKFADLVTDSPVISAIIDLKTLKNLKDNYGISKQQAFDLLRSDPRVLREFINQNNPIIRNRIENLIMQCRLGGGGSGGGGSGYPYDVPDYAGYPYDVPDYAGSYPYDVPDYAAQK

SEQ ID NO: 21 - protein, 441 aa, Artificial Sequence
  PRVA VP6 modified
MEVLYSLSKTLKDARDKIVEGTLYSNISDLIQQFNQMIVTMNGNDFQTGGIGNLPIRNWNFDFGLLGTTLLNIDANYVENARTTIEYFIDFIDNVCMDEMARESQRNGIAPQSEALRKLSGIKFKRINFDNSSDYIENWNLQNRRQRTGFVFHKPNILPYSASFTLNRSQPAHDNLMGTMWINAGSEIQVAGFDYSCAFNAPANIQQFEHVVPLRRALTTATITLLPDAERFSFPRVINSADGATTWYFNPVIIRPSNVEVEFLLNGQIINTYQARFGTIIARNFDTIRLSFQLVRPPNMTPAVANLFPQAPPFIFHATVGLTLRIESAVCESVLADASETLLANVTAVRQEYAIPVGPVFPPGMNWTELITNYSPSREDNLQRVFTVASIRSMLIKGGGGSGGGGSGYPYDVPDYAGYPYDVPDYAGSYPYDVPDYAAQC

SEQ ID NO: 22 - protein, 370 aa, Artificial Sequence
  PRVA VP7 modified
MYGIEYTTVLTFLISLVFVNYILKSVTRTMDFIIYRFLLVIVVLAPLIKAQNYGINLPITGSMDTPYMNSTTSETFLTSTLCLYYPNEAATEIADTKWTETLSQLFLTKGWPTGSVYFKGYADIASFSVEPQLYCDYNIVLMKYDGNLQLDMSELADLILNEWLCNPMDIMLYYYQQTDEANKWISMGTSCTIKVCPLNTQTLGIGCSTTDINSFETVANAEKLAITDVVDGVNHKLDVTTSTCTIRNCKKLGPRENVAVIQVGGPNILDITADPTTAPQTERMMRINWKRWWQVFYTIVDYVNQIVQVMSKRSRSLDSAAFYYRVGGGGSGGGGSGYPYDVPDYAGYPYDVPDYAGSYPYDVPDYAAQK

SEQ ID NO: 23 - protein, 361 aa, Artificial Sequence
  PRVA NSP2 modified
MAELACFCYPHLENDSYKFIPFNNLAIKCMLTAKVDKKDQDKFYNSIVYGIAPPPQFKKRYNTSDNSRGMNYVTTMFNKVAILICEALNSIKVTQSDVANVLSRVVSVRHLENLVLRKENHQDVLFHSKELLLKSVLIAIGQSKEIETTATAEGGEIVFQNAAFTMWKLTYLDHKLMPILDQNFTEYKITLNEDKPISDVCVKELVAELRWQYNRFAVITHGKGHYRVVKYSSVANHADRVFATYKNNAKSGNVTDFNLLDQRIIWQNWYAFTSSMKQGNTLDVCKKLLFQKMKQEKNPFKGLSTDRKMDEVSHVGIGGGGSGGGGSGYPYDVPDYAGYPYDVPDYAGSYPYDVPDYAAQC

SEQ ID NO: 24 - protein, 219 aa, Artificial Sequence
  PRVA NSP4 modified
MDKLADLNYTLSVITLMNDTLHSIIQDPGMAYFPYIASVLTVLFTLHKASIPTMKIALKTSKCSYKVIKYCMVTIINTLLKLAGYKEQVTTKDEIEQQMDRIIKEMRRQLEMIDKLTTREIEQVELLKRIHDKLAARSVDAIDMSKEFNQKNIRTLDEWESGKNPYEPSEVTASMGGGGSGGGGSGYPYDVPDYAGYPYDVPDYAGSYPYDVPDYAAQC

SEQ ID NO: 25 - DNA, 1612 bp, Artificial Sequence
  Promoter Sequence
attagctggttaataggtataataataataataataataataacaatagcaaattatagtagaaacgctattctatttctattttaatttttgatttgattttttagggtagcacactagacacgcttctgaaaagatgtgtgtatgtgtctcttcttgtttgacagtttgatatgactaagtgtctattttctgtttctgaagttctcccccataaaaataacacaactgacaacaacacatgtcaaatcccgtcatgatcccactcccggtctccaggtccccaatgctcaaacacccatgtcttttatatcttttgcgctgcccattttacaaagaaaccttgtacctttaaaaagtgatatgacgaaaaaaagaacaaattaaaaaacagtggggccctccaaagaatcagtcaataatcacaaaacttacattacagcacggcacagagcgcatgccgcatagtagaaggggagctacagaagagaaaaaaaaaaaaaaaaaacaaaaaaaaaaaaactaaggtaacgtaaagtaagtataataaaaataatctcatgagcaccaccaccgctcgccgcgatatacaaggtgggatgggatgggattattccaggatgcggcgcggcgcggcgtaggaaagcagtagcaagcccggtactagactgtactagactgtactaactaaccccagccgatcgccaatggaacaatggcatgcggggacggcccactgtgttccgcttacacgtgatcaaattcggccgcgtactccccgggttcggacaggtatggtccgtgcgcacccggtgcactccaccccgcgcgcggcccactgccttccggaaaaccaaaccaaaaaccaaaaccaaaaccaaaaccaaaaccaaagaaaccaaaacacatacctaaaccacccagaaaagcccgcgcaagaaaccgtttctccccctctctctctgccccgggtttccaggtattctcactcaaggcatcccaattccaggcttcgcttctagcagcacctgtggtgctgcttgctggtgtggtaccagcgtttctttacgcctggccactgggcactgtgcgccgaaacgcgcaagaacagtagtagtagtagtggcacattagcaagcttttctgctgtgaatgtgattgatggatggatgattggcactccatccgttccttgcttacgcaagctggcatatggcaatgtgtatgtgtgtgccccgaagtataggcagctaagcatatgatagttggctcgctctgcctttgcagtgtcagatatacacagtatataaggagttgcctgaatcttgtcagccggttcttagaagtcttcagaaacactttcgattcgggcacgttgtataaataagtgtgttgtgtttcagtttggagagagtgttgttattactattattattattcgtgtgtatgtccatttgaaagtatatatatatatatatatgtatatatgtctggttagtttgtttgtctataacaaccaaaaggtaaaatagtaatagtctggagaccctaaaagtataggattccaagcaaactagaaaaaaccaaaaaaaacatcaagaacaatcaagaa

SEQ ID NO: 26 - DNA, 3193 bp, Artificial Sequence
  Promoter Sequence
atctttcagttctcgatgagtatgtgtgtttatttttttttttttttgccaaattctgctctttcctaaatttcaagtgttaagcgcttttgtctcttcaccttttccaactacaagcgcaacataagaaaaggataataattctactaggaaacacaaacctcatatacctttcgagttagggaagaacaccttcacaatgataacacattgattcgagctattaaatacctttttctcaatcgaaatctcaagtaaaacagcgatgaaaacattacgtaactaatggtgttcaccactagaaatcatacccttcacactcgacttcaagtagtgaatggtgtagcaacaaagtccaaatgccaatatcaaccaagtaaccaaccgcgctactagagaaagacgctgttgctcggaccacaaatttccgctacacttttcacaactatactgaatatacaaaaaaacgtttgtgtgggtatggctggctaccaggtcgcctggttaaaccaagtcaacgtgatacatatgtacattccaacactaagcctaccctaagttttagctcacaggctaggctattattaacatacaagacaagggagaagcaaagaaaagaccaaccgaaagccaccagagcaccctgaactttgcggtgaacagaattccgcaacatatctgaggattccatgatctcggtttcctactccaaatgggaatcacccactgttgtccgtaaatatgactaaattcctaccttgattcctcacgaacaatcgcagtccgaaaagccgttccaaaagccagtccacagttcatcgattggtatgattattgtttttttgttcaaactgacacactaacggtgttgtatgcgaagagtgagcttacccttctcctctttgctagcactacttgcctacctacctactctactctactacgctgccatattgtctaacattctgctttctctatttctacctggcctggatggctccgtctcgcccgcctcacacgcacacacatacaaacacattccttccccctctcgcctgccccataataattaaactagttaacaaaaggctttacgtcttccgctacctctccaatctcatacgactctcctttcatccgacttacccaagaagatgcaggatctaagtcaaagatcctgcctgccctggctgttgtctactgtacacacgagatttggaaggccaggggactgtcgcatgattattcgccgggaacccggaagttccctgttcttacctttcaaactaggcagcaaaccaggtgaacacattttgatgtagtaggcctttgaagattcggggagctagctagccacgcgaatgtaacaacagaacatttatttttgtgggggtggaaaagtcgattatgatcttgcagcacagaaactgcgcaggggcttttttcatcttggagaagcaactggctaaattcgacacaaacaaaaactgaaaaatggaaaataaaaaatgaaaaagcaagctgaattcgaagaagggagaattccgccttctgcaaccacactaatggttggtagtcaatggatacgcattagaaggtgactattttatgagtcgatccccgcggaaatttaggaattttaaacttgggcttgatatacgtaggtatagtgtatctggtatttttgccaatggttcctgtgcccgtgcccatgtccgtgtgctcatgcgatgcactgtgactactgcttggcaaggcaaatcggcggcctgcggccgtactctaaagccggacaggcatggtggggtgccttttcggtccataagtgtagctagatagctagctagctacacgtatgttctgtgcgaagatggtaaaggttgcatacagtcaggctgctgtctttggacctggtctgacctgacctagcataatattgcatagcgtatacccacttggatggatgcaggagtgctacaaatcggcgaacagttctccttgtttgtttttggtcatagtcacgtccggctacacacaagatattgcacagtatgtgtagatacgagcgaaggtacggctggaaaacaggtgggttttggatgatagacagaaggcaagagggtgggcaagaagtcgggcacacaggcaagtattagcatggtcaaaccagattaaggtctaggccaagaacaattttcggccccccactgactcccgccaaaaaaaaaaaaaaaaaaaaaaacctaatttgacagtctcggaattttatctatctgttcctttcatttggtaactagtatcgtcactttttttttggaattggcaagctacctgcatggccgtataccgaatgaaccccagaaaatggtatagctaagctttcgtacgacggaaatatgtagagcgaagggttcttggtctgtgaaaggggttttctgtactacgtaaagaggggtcttcattatagcttttcaatggattcgaaatggaagtaagcgtaagagaatgaaaaacaagaaaatagttagaaataaaaaaaaaaaaaaacacttggctgtctgccggacttgtcttgtcttggtggaaatcattactccttggcgggcgatgaatgtgcttctgcttcttttcattaatacataccgtcattggtagtattctgatttcatttctccacaccattttttttgcgttagtcgtctgcggtatatgcgaccctggccgttcggtgcgccgagtgcgtttacctgtgtaggcgcttttgtaagagagaaactcgggtctgggtgggcctgcaacgtgacttagcaatacccgcgcgtcagatttaacatattcgggattcttccctccgtagtagaaagttgacaataagatgtccatactgttgttgataccttgctactctagctcattattacaaggacagccggttaattggaggcctaaatggtgtatcaaggtatataaagccaataactttcaatgttgagttgggctgtagttcaaggtggttgtagcttgtggaacatatatatgctattcaatatagctagaaagtgtaagctaaacagcaagctaaacagcaaacaaaagtctgtgtgtgttgtcttataattatatccttcagtcagtgttaataattaaagacagaattacgaaaaataaaacaggacgataatcagttttcata

SEQ ID NO: 27 - DNA, 2822 bp, Artificial Sequence
  Promoter Sequence
atctttcagttctcgatgagtatgtgtgtttatttttttttttttttgccaaattctgctctttcctaaatttcaagtgttaagcgcttttgtctcttcaccttttccaactacaagcgcaacataagaaaaggataataattctactaggaaacacaaacctcatatacctttcgagttagggaagaacaccttcacaatgataacacattgattcgagctattaaatacctttttctcaatcgaaatctcaagtaaaacagcgatgaaaacattacgtaactaatggtgttcaccactagaaatcatacccttcacactcgacttcaagtagtgaatggtgtagcaacaaagtccaaatgccaatatcaaccaagtaaccaaccgcgctactagagaaagacgctgttgctcggaccacaaatttccgctacacttttcacaactatactgaatatacaaaaaaacgtttgtgtgggtatggctggctaccaggtcgcctggttaaaccaagtcaacgtgatacatatgtacattccaacactaagcctaccctaagttttagctcacaggctaggctattattaacatacaagacaagggagaagcaaagaaaagaccaaccgaaagccaccagagcaccctgaactttgcggtgaacagaattccgcaacatatctgaggattccatgatctcggtttcctactccaaatgggaatcacccactgttgtccgtaaatatgactaaattcctaccttgattcctcacgaacaatcgcagtccgaaaagccgttccaaaagccagtccacagttcatcgattggtatgattattgtttttttgttcaaactgacacactaacggtgttgtatgcgaagagtgagcttacccttctcctctttgctagcactacttgcctacctacctactctactctactacgctgccatattgtctaacattctgctttctctatttctacctggcctggcctgcaggggctccgtctcgcccgcctcacacacatacattcctccccctctcgcctgccccataataattaaacaagttaacaaaaggcgttacctcttccgcatcctctccaatctcatacgattcccctttcatccgacttacccaacaagatacaggatctcagtgaaagatccttcctgccctccctgtctgttgtctactctacatgcgacttggaaggccaaaggactatcgcatgattattcgccgggaacccgcgagttccctgctcttttctttcaaaccaggcagcaaaccaggtgaacacactctgatgtagtgcagtccctaagtcctttgaagattcggggagctagctacccacgcgaatgtaacaaaagaacatttacttttgtggggggtggaaaagtcgattaggatcttgcagcacagaaactgcgcaggggtttttttcatcttggagaagcaactggctaaattcgacacaaacaaaaactgaaaaatggaaaataaaaaatgaaaaagcaagctgaattcgaagaagggagaattccgccttctgcaaccacactaatggttggtagtcaatagatacgcattagaaggttactattttatgagtcgatccccgcggaaatttaggaattttaaacttgggcttgatatacgtcggtatagtgtatctggtatttttgccaatggttcctgtgcccgtgcccgtgcccgtgtgctcatgcgatgcattgtgactactgcttggcaaggcaaatcagcggtctgcggccgtactctaaagccggacagacatggtggggtgccttttcggtccataagtgtaactagctagctacaagtatgttctgtgcaaagatggtaaaggttgcatacagtcaagttgctgtccttggacctggtctgacctgacctagcttaacattgcatagcgcatacccacttggatggatgcaggagtgttacaaatcggcgaacagttctccttgtttgtttttggtcatagtcacgtccggctacacacaagatatggcacagtatgtgtagatgcgagcgaagatacggctggaaaacaggtgggttttgaaggatagacagaaggcaagagggtgggcaaaaagtcgggcactcaggcaagtattggcatgatcaaaccagattaagttctaggccaagaacatatttcggcccactgactcccgccaaaaaaaaaaaaaaaaaaacctaatttgacagtctcggaattttatctatctgttcctttcctttggttactagtatcgtcatttaattttttttggaattggcaagctacctgcgtggctctataccgaatgaaccccgaccctggccgttcggtgcaccgagcgcgtttacgtatgtacacgcttttgtaagaaagaaactcgggtctgcgtgggcctgcaacgtgacttagcaatacccgcgcgtcagatttaacatattccggattcttccctccgtactagaaagttgacaataagatgtccatactgttgttgataccttgctactctagctcattatgacaaggacaatcggttaattggaggtctaaatggtgtatcaaggtatataaaggcagtaactttcaatgttgagttgggctgtagttcaaggtggttgtagcttctggaacatatatatgctattcaatatagctggaaagtgtaagctaaacagcaaacaaaagtctctgtgtgttgtcttataattatatccttcagtcagtgttaatgattaaagacagaattacgaaaaaataaaacaggacgataatcagttttcata

SEQ ID NO: 28 - DNA, 2361 bp, Artificial Sequence
  Promoter Sequence
ttctagttgttggttgttgtttttggagttagtttgtagctagcgttttaatgcttgattgctggtgtttgtagttcaaaagatattataattgagaaacaatgggacaagtaacgagatgggttaggattggattgttgtaaattgggtcaatttgctggtccttatatacagattgaacgacgaagatgttggatctcgttcctcaaagtgttgccgatatgttggggcaagcaaacaagcaggcaggcaggctgccaggcgggcagccagtcagtcaatcagtcagtcagttagccagccatcagctgtccaatattagctaccttagcttaaggcaaacacaaacacacacggtttccgcgcgggaagcccaaagcccagtaaacgcagcagtacctgcgtgagcatgtaagaagcactaagacgtgatcttgatcttgaacttgaactactaccactgcagcgcctgctgcccgtctgtttgggttttggcttggcttttgggcttcgaaggaagcattgggctttcacagacaaccgcgtagacccctcacttgtggtcaaaaaagaacccttcgaaaaacaaaacagggaaaaatggaaaaaaaaaccgtatgagaaaaaggtatggttccactcaatcaccggccatctcgcactggtgatctcacaaagaggaagtgtttgagaacgaataaaaaaaaaatccgagcccagtgcacgccctgcagtaggtgttgtagcagtgggtgggtgggtattgcttaaacggaaaaacttacgagaatcaacggaaaaaaaaaaaattaacaaaaccaaaccaaacaaacctgggtctgagataggatagggcaggcagtgagtatttggttggttggtgggtgggtggttacgagccagcagcaccagggaaacgagaggagtaatgagaaatgaagactgcgaaccacttctggaagaaacaacatacacggacgagagagatcaggttacttaaaagaggaagctgcgggccagctgatttcggactacaggaagctgaaggtgtgttttcaaggaatccggagaggaggccgccagaggctaggccagaggctaggccagaggcgaggcaaaaggcgaagctaaaaaaaaggaatccggacaactctcctacccccctattatttgcaatactggaacccaggctggtgagcgagcgcgagtacgtgtgctctctttagtggaacccaccggaactcactggaagccagcttcctgtcacgtgacaggaagccagaacccctcctcgcctctccttgccccctccaataagaatcgttacataccaacaaaaacacccacacccacacacccacaactacactcctatcgcacaggaagccaggactccgaacagctagctgcttgcttgctgctcgcttgttgcctgcttgctgcctgcctacaaaccaaccaaccttaaaacgctctctgttgccttctaccaccgcaccagcccagaaccccacgcaccaccagtggcaccatctccgagaatcgtccctcccccggacgtccttaagtcataccactgcaaccactcaactctgaaacgcacctggcttccacttttctgcacaggctcccagctaccacatttccccagaaattctcccatccatgcccctacaattaaaaatcaccttcccacaactccgcagagaaccagagcacatgattggcatccctagcccccatcactactggattctcggttgttacactgcaacgtgcattcttctatgccactctacgctgtggtttcctagtgtttccaaaccagatttcttggtgtttcgtggcgatttcctgatttcctgatttcctggtttcctgattccttccttccttcctggtggtgtctctctttctgtgtgttttttctttctttctttctttattgcttgcttgctgccatgctgctaccttaattcttcttttttcctaccctaattcttgtaatttgtaatgaaagacaagaccaccatacatacacacatacatgatatcgtggcattgtgccatggcaccatggaatgggatatgggatatatggcaccgtctttacttagctccgaagctttgaggatccgaaatttggtatataaggggtggaacattgtcagaaagaagcaatccatccaagttgtcttgatgtcagttttagtttagatgtttcagcatttagttttagtgttagcttgtctactctccccaaggaacaaggacactttttcatacaaagtcttctgtttgcacgaatttaattgcgtacgttctcccccacacacaactcaactcagataattttaaaag

SEQ ID NO: 29 - DNA, 250 bp, Artificial Sequence
  Terminator Sequence
tcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctc

SEQ ID NO: 30 - DNA, 250 bp, Artificial Sequence
  Terminator Sequence
aagtgtcgaacttgaaaatatcaatggaaaagaatcaatcagtggattcgaagaaagttctactttctaactcagcttcacccgtacctctagtcgaaaccacattggagctaaacattagaagccagttaggagatttcttttcttcaagggtaggatgttagtgtaatatatttatattcatacatatatatatatgtatttacgagtgttttaaggtgatatgaaattccttatcaaacctatcaat

SEQ ID NO: 31 - DNA, 252 bp, Artificial Sequence
  Terminator Sequence
atagtttgtccaaatctgtcgttaacaaaaactatacaaaccagatttttatgagctttctgtaaaaaaataacaatataaatgttttcaaaacgagtaagcagtctaactctgcttggaacctatatatacgcgtctcttgtaaacaccatatgtagtgtcagaataacttgtcagctcaactgaaatagtactaggatttataaaaaaaatataataaccgggtaacaaattttgttctggtcgtttttg

SEQ ID NO: 32 - DNA, 190 bp, Artificial Sequence
  Terminator Sequence
atccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaag

SEQ ID NO: 33 - DNA, 188 bp, Artificial Sequence
  Terminator Sequence
gcgaatttcttatgatttatgatttttattattaaataagttataaaaaaaataagtgtatacaaattttaaagtgactcttaggttttaaaacgaaaattcttgttcttgagtaactctttcctgtaggtcaggttgctttctcaggtatagcatgaggtcgctcttattgaccacacctctaccgg

SEQ ID NO: 34 - DNA, 2805 bp, Artificial Sequence
  PRVA VP2 modified sequence
atggcatacagaaagagaggtgcaaaaagagaaaactcatcacaacaaaacgaaagattacaagaaaaggaagtagaaaagaacattgatgcatctatggaaaataaggctaacaataagaaacaacaattgccagataaggttttgtcacaaaaggaagaaatcatcactgatgctcaagatgatgttaagatcacagatgaagttaagaaatcttcaaaagaagaatctaagcaattgttggaaattttgaagactaaagaagatcatcaaaaagaaattcaatacgaaattttacaaaagactatcccaacattcgaaccaaaggaatcaatcttgaagaaattggaagatatcaagccagaacaagctaagaaacaaactaagttgtttagaatcttcgaaccaagacaattgccaatctatagagctaatggtgaaagagaattgagaaacagatggtactggaagttgaagagagatacattgccagatggtgactatgatgttagagaatactttttgaatttgtacgatcaaattttaatcgaaatgccagattatttgttgttgaaggatatggcagttgaaaataagaactctagagatgctggtaaagttgttgattctgaaactgcatcaatctgtgatgctatcttccaagatgaagaaacagaaggtgttattagaagattcattgcagatatgagacaacaagttcaagctgatagaaacgttgttaactacccatctatcttgcatccaatcgatcatgcttttaatgaatactttttgaaccatcaattggttgaaccattgaacaacgaaatcattttcaattacattccagaaagaattagaaatgatgttaattatattttgaatatggatatgaatttgccatctactgctagatacattagaccaaatttgttgcaagatagattgaatttgcatgataacttcgaatcattgtgggatactatcactacatctaactacatcttggcaagatcagttgttccagatttgaaggaaaaggaattagtttctacagaagctcaaatccaaaagatgtcacaagatttgcaattggaagcattgactatccaatctgaaacacaattcttggctggtattaattcacaagctgcaaacgattgtttcaagactttgatcgctgcaatgttgtctcaaagaacaatgtcattggaattcgttactacaaactacatgtctttgatttcaggcatgtggttgttgactgttattccaaacgatatgttcttgagagaatctttggttgcttgtgaattggcaattattaacactatcgtttacccagctttcggtatgcaaagaatgcattacagaaatggtgacccacaaacaccatttcaaattgcagaacaacaaatccaaaacttccaagttgctaactggttgcatttcatcaacaacaacagattcagacaagttgttattgatggtgttttgaaccaaactttgaacgataacatcagaaatggtcaagttattaatcaattgatggaagctttgatgcaattgtctagacaacaattcccaacaatgccagttgattacaagagatcaatccaaagaggtatcttattgttgtcaaacagattgggtcaattggttgatttgactagattgttgtcatacaactacgaaactttgatggcttgtatcacaatgaacatgcaacatgttcaaacattgactacagaaaagttgcaattgacttctgttacatcattgtgtatgttgatcggtaacactacagttattccatctccacaaactttgttccattactacaacgttaacgttaacttccattcaaattacaatgaaagaattaatgatgctgttgcaatcatcacagctgcaaacagattgaatttgtaccaaaagaaaatgaaatctattgttgaagaatttttaaaaagattgcaaatttttgatgttccaagagttccagatgatcaaatgtacagattgagagatagattgagattgttgccagttgaaagaagaagattggatatttttaatttgatcttgatgaatatggaacaaatcgaaagagcatctgataagatcgctcaaggtgttattatcgcatacagagatatgcaattggaaagagatgaaatgtacggttacgttaacatcgctagaaatttggatggttaccaacaaattaatttggaagaattgatgagaactggtgactatggtcaaatcacaaacatgttgttgaacaaccaaccagttgctttggttggtgcattgccattcgttactgattcttcagttatttctttgatcgctaagttggatgcaactgttttcgctcaaatcgttaagttgagaaaagttgatacattgaaaccaatcttgtacaagattaattctgattcaaatgatttttacttagttgcaaattatgattggatcccaacatcaactacaaaggtttacaagcaagttccacaaccattcgatttcagagcttctatgcatatgttgacttcaaatttgacttttacagtttactctgatttgttggcatttgtttcagctgatacagttgaaccaattaatgctgttgcattcgataacatgagaatcatgaacgaattgggtggtggtggttctggtggtggtggttcaggttatccatacgatgttccagattatgctggttatccatacgatgtccctgattatgctggttcttatccttacgatgtccctgactatgctgctcaaaagtga

SEQ ID NO: 35 - DNA, 2463 bp, Artificial Sequence
  PRVA VP4 modified sequence
atggcttctttgatctatagacaattgttgactaactcatacacagttaatttgtctgatgaaatccaagaaatcggttctgctaagtctcaagatgttactattaatccaggtccatttgctcaaacaggttacgcaccagttaattggggtgcaggtgaaactaatgattctactacagttgaaccattgttagatggtccataccaaccaactacttttaatccaccaacttcttattgggttttgttagctccaacagttgaaggtgttattatccaaggtacaaacaacactgatagatggttggcaactatcttgatcgaaccaaacgttcaaactacaaacagaatctataatttgtttggtcaacaagttacattgtcagttgaaaacacatctcaaactcattggaagtttattgatgtttcaactacaactccaactggttcttacacacaacatggtccattattttcaactccaaagttgtacgctgttatgaaattttctggtagaatctatacatacaatggtacaactccaaatgcaacaactggttactactcaacaactaactacgatactgttaacatgacatctttctgtgatttctacattattccaagaaaccaagaagaaaagtgtactgaatacatcaaccatggtttaccaccaatccaaaacacaagaaacgttgttccagtttcattgtctgctagagaaattgttcatactagagcacaagttaacgaagatatcgttgtttcaaagacatctttatggaaagaaatgcaatgtaacagagatatcactatcagattcaaattcgatagaacaattattaaggctggtggtttgggttataaatggtcagaaatctcttttaaaccaatcacttaccaatacacatacgctagagatggtgaacaaattactgcacatacaacttgttctgttaacggtgttaacaacttctcatacaacggtggttctttgccaacagatttcgctatctcaagatacgaagttattaaagaaaattcttttgtttacattgattattgggatgattcacaagcttttagaaacatggtttacgtcagatctttggctgcaaatttgaatacagttacttgtacaggtggttcatactcttttgctttgccattaggtcattatccagttatgacaggtggtactgtttcattacatccagcaggtgttacattgtctactcaattcactgatttcgtttcattgaactctttgagattcagattcagattaactgttggtgaaccatcattttctatcactagaacaagagtttctagattgtacggtttaccagctgcaaatccaaacaaccaaagagaatactacgaaatctcaggtagattttcattgatctctttggttccatctaacgatgattaccaaactccaatcatgaactcagttacagttagacaagatttggaaagacaattgggtgaattgagagatgaattcaattcattgtctcaacaaatcgctatctctcaattgatcgatttggcattgttgccattggatatgttctcaatgttctctggtattaaatcaactatcgatgctgcaaagtctatggctacaaacgttatgaagagattcaaaagatcaaatttggcttcttcagtttctactttgacagatgcaatgtcagatgctgcatcttcaatctctagatcttcatctatcagatcaattggttcatctgcttctgcatggactgaagtttcaaattctatcgctgatgtttcaacaactgttgatacagtttctactcaaacagctactatcgcaaagagattgagattgaaggaaatcgcaacacaaactgatggtatgaacttcgatgatatctctgctgcagttttgaagactaagatcgataagtcagttcaaattactccaaatacattgccagaaatcgttactgaagcttctgaaaagtttattccaaacagaacatacagagttattaataacgatgaagttttcgaagttggtatggatggtaaatttttcgcttacagagttgatactttcgatgaaatcccattcgatgttcaaaagttcgctgatttggttactgattcaccagttatttctgcaatcatcgatttgaagacattgaaaaatttgaaagataattatggtatctcaaagcaacaagctttcgatttgttaagatctgatccaagagttttgagagaattcattaaccaaaacaacccaatcatcagaaatagaattgaaaatttgattatgcaatgtagattgggtggtggtggttctggtggtggtggttccggttacccatatgatgttccagattacgctggttatccttatgatgttccagattatgcaggttcctatccatacgacgttccagattatgctgcacaaaaataa

SEQ ID NO: 36 - DNA, 1326 bp, Artificial Sequence
  PRVA VP6 modified sequence
atggaagttttgtactctttgtccaagactttgaaggatgccagagataagatcgttgaaggtactttgtactctaacatttctgatttgatccaacaattcaaccaaatgattgttaccatgaacggtaacgacttccaaactggtggtatcggtaacttgccaattagaaactggaacttcgacttcggtttgttgggtaccactttgttgaacatcgatgctaactacgttgaaaacgctagaaccactatcgaatacttcatcgacttcatcgataacgtttgtatggatgaaatggctagagaatctcaaagaaacggtatcgctccacaatctgaagccttgagaaagttgtccggtatcaagttcaagagaattaacttcgacaactcttccgattacatcgaaaactggaacttgcaaaacagaagacaaagaaccggtttcgttttccacaagccaaacattttgccatactctgcttccttcaccttgaacagatcccaaccagctcacgacaacttgatgggtactatgtggatcaacgctggttctgaaattcaagttgctggtttcgattactcctgtgctttcaacgctccagctaacatccaacaattcgaacacgttgttccattgagaagagctttgaccactgctaccatcactttgttgccagacgctgaaagattctctttcccaagagttattaactccgctgatggtgctaccacttggtacttcaacccagttatcattagaccatctaacgttgaagttgaatttttgttgaacggtcaaatcattaacacctaccaagctagattcggtactatcattgctagaaacttcgacaccatcagattgtccttccaattggttagaccaccaaacatgactccagctgttgctaacttgttcccacaagctccaccattcatcttccacgctaccgttggtttgactttgagaattgaatctgctgtttgtgaatccgttttggctgatgcttctgaaaccttgttggctaacgttactgctgttagacaagaatacgctatcccagttggtccagttttcccaccaggtatgaactggaccgaattgattactaactactctccatccagagaagacaacttgcaaagagttttcaccgttgcttctatcagatccatgttgatcaagggtggtggtggttctggtggtggtggttccggttacccatacgacgttccagattacgctggttacccatacgatgttccagactacgccggttcctacccatacgatgttccagactacgctgcccaaaagtaa

SEQ ID NO: 37 - DNA, 1113 bp, Artificial Sequence
  PRVA VP7 modified sequence
atgtacggtatcgaatacactacagttttgacatttttgatttctttggttttcgttaactacatcttgaagtcagttactagaacaatggattttattatctatagatttttgttagttattgttgttttggctccattgattaaagcacaaaactacggtattaatttgccaatcactggttctatggatacaccatacatgaactctactacatcagaaacatttttgacttcaacattgtgtttgtactacccaaacgaagctgcaactgaaattgctgatacaaagtggactgaaacattgtctcaattgtttttaactaaaggttggccaacaggttcagtttacttcaagggttacgctgatatcgcatctttttcagttgaaccacaattgtactgtgattacaacatcgttttgatgaaatatgatggtaatttgcaattggatatgtctgaattggctgatttgatcttgaacgaatggttgtgtaacccaatggatatcatgttgtactactaccaacaaactgatgaagcaaataagtggatctctatgggtacttcatgtacaattaaagtttgtccattgaatactcaaacattgggtattggttgttctactacagatatcaactcattcgaaactgttgctaacgcagaaaagttggctatcacagatgttgttgatggtgttaaccataagttggatgttactacatcaacttgtacaatcagaaactgtaagaaattgggtccaagagaaaatgttgcagttattcaagttggtggtccaaatattttggatattactgctgatccaactacagcaccacaaacagaaagaatgatgagaattaattggaagagatggtggcaagttttctatactatcgttgattacgttaaccaaatcgttcaagttatgtctaagagatctagatcattggattcagctgcattttactatagagttggtggtggtggttctggtggtggtggttcaggttacccatatgatgttccagattacgctggttatccttatgatgttccagattatgcaggttcctatccatacgacgttccagattatgctgcacaaaaataa

SEQ ID NO: 38 - DNA, 22 bp, Artificial Sequence
  P1 primer
caaatgttgtggctgcacctac

SEQ ID NO: 39 - DNA, 21 bp, Artificial Sequence
  P2 primer
caaacgtatgcgtacgaaatc

SEQ ID NO: 40 - DNA, 24 bp, Artificial Sequence
  P3 primer
ctcaggatataggaaacatgaagg

SEQ ID NO: 41 - DNA, 20 bp, Artificial Sequence
  P200 primer
aagaagacttagccatggca

SEQ ID NO: 42 - DNA, 25 bp, Artificial Sequence
  P204 primer
gaagaaccaataatgttaatatgac

SEQ ID NO: 43 - DNA, 24 bp, Artificial Sequence
  P162 primer
tatgaaaactgattatcgtcctgt

SEQ ID NO: 44 - DNA, 20 bp, Artificial Sequence
  P532 primer
tatgtctgctttggagcaca

SEQ ID NO: 45 - DNA, 21 bp, Artificial Sequence
  P512 primer
gttgacgttaacatgattgga

SEQ ID NO: 46 - DNA, 20 bp, Artificial Sequence
  P118 primer
atctttcagttctcgatgag

SEQ ID NO: 47 - DNA, 22 bp, Artificial Sequence
  P375 primer
cggcaaaaaaacgcaggatctg

SEQ ID NO: 48 - DNA, 27 bp, Artificial Sequence
  P172 primer
attgagaatcacctaaaccaaacacgg

SEQ ID NO: 49 - DNA, 20 bp, Artificial Sequence
  P533 primer
actgcagttaagattccagc

SEQ ID NO: 50 - DNA, 20 bp, Artificial Sequence
  P514 primer
gccagaaatacaacacctct